(19) United States
(12) Patent Application Publication
(10) Pub. No.: US 2023/0219912 A1
(43) Pub. Date: Jul. 13, 2023

(54) HALOGENATED BENZOTHIADIAZINES FOR THE TREATMENT OF CANCER

(71) Applicant: BOARD OF REGENTS OF THE UNIVERSITY OF NEBRASKA, Lincoln, NE (US)

(72) Inventors: Paul Trippier, Papillion, NE (US); Bader Huwaimel, Omaha, NE (US)

(21) Appl. No.: 17/925,765

(22) PCT Filed: May 19, 2021
(86) PCT No.: PCT/US21/33235

§ 371 Date: Nov. 16, 2022

Related U.S. Application Data
(60) Provisional application No. 63/027,180, filed on May 19, 2020.

(51) Int. Cl. C07D 285/08

(52) U.S. Cl. CPC C07D 285/08

(57) ABSTRACT

Provided herein are compounds and methods for modulating the mitochondrial respiratory complex II (CII) and vascular endothelial growth factor (VEGF) pathways. More particularly, provided are inhibitors of the mitochondrial respiratory complex II (CII) and vascular endothelial growth factor (VEGF) pathways and the uses of such inhibitors in regulating diseases and disorders, e.g., to treat cancer.

### CROSS-REFERENCE TO RELATED APPLICATION

The benefit of priority to U.S. Provisional Patent Application No. 63/027,180 filed May 19, 2020 is hereby claimed and the disclosure is incorporated herein by reference in its entirety.

### STATEMENT OF U.S. GOVERNMENT SUPPORT

This invention was made with government support under grant number R01 CA226436 awarded by the National Institutes of Health. The government has certain rights in the invention.

### BACKGROUND

Targeting angiogenesis is an appealing strategy for developing selective antineoplastic agents. Angiogenesis is a physiological process which is tightly regulated by the balance between angiogenic and antiangiogenic factors in normal tissues. Vascular permeability factor (VPF) or vascular endothelial growth factor (VEGF) is one of the most characterized angiogenic factors. It plays a critical role in the vascular system in both pathological angiogenesis, such as cancer, and normal physiological functions including bone formation, hematopoiesis, wound healing, and development. The main function of VEGF is to modulate blood vessel permeability and remodeling, as well as endothelial cell survival, proliferation and migration. In tumor cells, VEGF is highly overexpressed which induces other proangiogenic factors and leads to the formation of new blood vessels. Vascular endothelial growth factor and its receptors are dysregulated by activation, overexpression, or mutation that leads to tumorigenesis and metastasis by initiating downstream signaling transduction pathways resulting in angiogenesis, vascular permeability enhancement and tumor development.

Targeting cancer metabolic pathways is another appealing strategy for the development of selective antineoplastic agents. This approach may provide a therapeutic advantage that can help overcome drug resistance, enhance the specificity of cancer cell targeting, increase the potency of existing treatments and overcome, or attenuate, adverse effects. Much emerging evidence implicates mitochondrial metabolism as a key driver of tumor growth. Mitochondria are crucial not only for energy production, but also for regulating essential steps of cell apoptosis and reactive oxygen species (ROS) generation; contributing to many processes within the cell, most notably in electron transport-linked phosphorylation, central carbon metabolism (CCM), and the biosynthesis of intermediates for cell growth. Mitochondria participate in nearly all aspects of cell function including growth (including aberrant cancerous growth), inflammation, metabolic signaling, cell death, and transformation. Furthermore, cancer cells possess a more hyperpolarized mitochondrial membrane potential than non-cancerous cells, with increasing hyperpolarization directly corresponding to more invasive and aggressive cancers. Adenosine triphosphate (ATP) synthesis is crucial to the survival of all cells; however, as cancerous cells are continually dividing, they require more energy than non-cancerous cells. These two facets of cancer cell mitochondria provide for the possibility of selectively targeting mitochondria in cancer cells over healthy cells.

Many traditional chemotherapeutic agents suffer from a narrow therapeutic index that presents as a major drawback in treating cancer, in addition to a lack of specificity, severe side effects, and development of drug resistance. Therefore, compounds that inhibit antiangiogenesis and/or target mitochondrial oxidative metabolism are appealing targets for treating tumors and assisting in overcoming the side effects of the chemotherapeutic agents.

### SUMMARY

Provided herein are compounds and methods for modulating the mitochondrial respiratory complex II (CII) pathway, and which can treat or prevent a disease or disorder associated with the mitochondrial respiratory complex II (CII) in a subject. Also provided are compounds and methods for inhibiting the vascular endothelial growth factor (VEGF) pathway, and which can treat or prevent a disease or disorder associated with the vascular endothelial growth factor (VEGF) in a subject.

In one aspect, the disclosure provides compounds having the structure of Formula I:

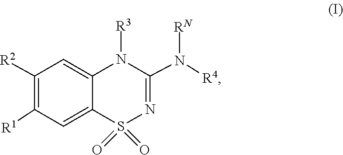

wherein R^{N} is H or C₁₋₆ alkyl; R¹ and R² are each independently H, halo, CN, C₁₋₆ haloalkyl, isothiocyanate, or —OSO₂R^{S}, wherein R^{S} is C₁₋₃ alkyl, C₁₋₃ haloalkyl, or phenyl optionally substituted with one of halo, C₁₋₃ alkyl, or NO₂; R³ is H, C₁₋₆ alkyl, C₃₋₁₀ cycloalkyl, or C₆₋₁₀ aryl; R⁴ is C₀₋₆alkylene-C₃₋₆ cycloalkyl, C₀₋₆alkylene-3-12 membered heterocycloalkyl having 1-3 ring heteroatoms selected from O, S, and N, C₀₋₆alkylene-C₆₋₁₀ aryl, or C₀₋₆alkylene-5-10 membered heteroaryl having 1-4 heteroatoms selected from N, O, and S, and said cycloalkyl, heterocycloalkyl, aryl, and heteroaryl are optionally substituted with one or more R⁵; and each R⁵ is independently halo, C₁₋₆ alkyl, C₁₋₆ haloalkyl, C₁₋₆ alkoxy, C₁₋₆ haloalkoxy, S—C₁₋₆ haloalkyl, or SO₂N(R^{N})₂, with the proviso that the compound is not: 3-(cyclopentylamino)-7-fluoro-4H-benzo[e][1,2,4]thiadiazine 1,1-dioxide; 6-chloro-3-(cyclopentylamino)-4H-benzo[e][1,2,4]thiadiazine 1,1-dioxide; or 6-chloro-3-(phenylamino)-4H-benzo[e][1,2,4]thiadiazine 1,1-dioxide.

Also provided are compounds as disclosed in Table 1. Further provided are methods of using the compounds of Formula I, Table 1, and Table 2 for modulating the mitochondrial respiratory complex II (CII) pathway and/or inhibiting the vascular endothelial growth factor (VEGF) pathway, and/or for treating or preventing diseases and disorders as disclosed herein. Other aspects of the disclosure include a compound as disclosed herein for use in treating or preventing a disease or disorder associated with the mitochondrial respiratory complex II (CII) pathway and/or the vascular endothelial growth factor (VEGF) pathway, and the use of a compound as disclosed herein for use in the preparation of a medicament for treating or preventing a disease or disorder associated with the mitochondrial respiratory complex II (CII) pathway and/or the vascular endothelial growth factor (VEGF) pathway.

### BRIEF DESCRIPTION OF THE DRAWINGS

FIG. 1 shows mitochondria respiratory complex II; succinate dehydrogenase (SDH), is a member of the respiratory chain and TCA cycle, wherein it catalyzes the oxidation of succinate to fumarate.

FIG. 2 shows structures of complex II inhibitors 1-9.

FIGS. 3A-3D show percentage inhibition of mitochondrial complex II relative to DMSO control at 100 μM concentration of diazoxide derivative. FIG. 3A shows complex II inhibitory activity for diazoxide derivatives with 7-fluoro substitution. FIG. 3B complex II inhibitory activity for diazoxide derivatives with 6-chloro substitution. FIG. 3C shows complex II inhibitory activity for diazoxide derivatives with 7-bromo substitution. FIG. 3D shows complex II inhibitory activity for diazoxide derivatives with a saturated ring. Values represent the mean±SD of n=4 experiments.

FIGS. 4A-4D show cytotoxic effect of diazoxide derivatives (100 μM, 48 hr treatment) in 22Rv1 prostate cancer cells. FIG. 4A shows the cytotoxic effect of diazoxide derivatives with 7-fluoro substitution. FIG. 4B shows the cytotoxic effect of diazoxide derivatives with 6-chloro substitution. FIG. 4C shows the cytotoxic effect of diazoxide derivatives with 7-bromo substitution FIG. 4D shows the cytotoxic effect of diazoxide derivatives with a saturated ring. Values represent the mean±SD of n=3 experiments.

FIG. 5 shows the cytotoxic effect of selected diazoxide derivatives on triple negative breast cancer (TNBC) MDA-MB 468 cells. Values represent the mean of three separate experiments performed in triplicate.

FIGS. 6A and 6B show the cytotoxic effect of diazoxide derivatives at 20 μM concentration in HUVEC cells in the presence of VEGF (10 ng/mL). FIG. 6A shows the cell proliferation inhibition of VEGF (10 ng/mL) treated HUVEC cells by diazoxide derivatives previously synthesized. FIG. 6B shows the cell proliferation inhibition of VEGF (10 ng/mL) treated HUVEC cells by diazoxide derivatives newly synthesized in this manuscript. Values represent the mean±SEM of n=3 experiments.

FIG. 7 shows Western blots from HUVEC cells treated with VEGF (10 ng/mL) and selected DZX derivatives (20 μM). The compounds downregulate expression of phosphorylated vascular endothelial growth factor receptor-2 (VEGFR2).

FIGS. 8A and 8B show the cytotoxic effect of potent diazoxide derivatives (50 μM, 72-hour treatment). FIG. 8A shows Cytotoxic effect of diazoxide derivatives on human embryonic kidney (HEK293) cells. FIG. 8B shows the cytotoxic effect of diazoxide derivatives on triple negative breast cancer (MDA-MB-468) cells. Values represent the mean±SD of n=3 experiments.

### DETAILED DESCRIPTION

Provided herein are compounds that inhibit the mitochondrial respiratory complex II (CII) pathway, and can treat or prevent a disease or disorder associated with the mitochondrial respiratory complex II (CII) in a subject. Also provided are compounds that inhibit the vascular endothelial growth factor (VEGF) pathway, and can treat or prevent a disease or disorder associated with the vascular endothelial growth factor (VEGF) in a subject. Further provided are compounds that inhibit the mitochondrial respiratory complex II (CII) pathway and inhibit the VEGF pathway. Any of the compounds herein can be useful in the treatment of a variety of diseases and disorders, including but not limited to cancer, neurodegenerative diseases and disorders, and eye diseases and disorders. Any diseases or disorders known to be treated, prevented or mediated by inhibition of the mitochondrial respiratory complex II (CII) pathway or the VEGF pathway can benefit from treatment with the compounds of the disclosure.

### CII Inhibitors

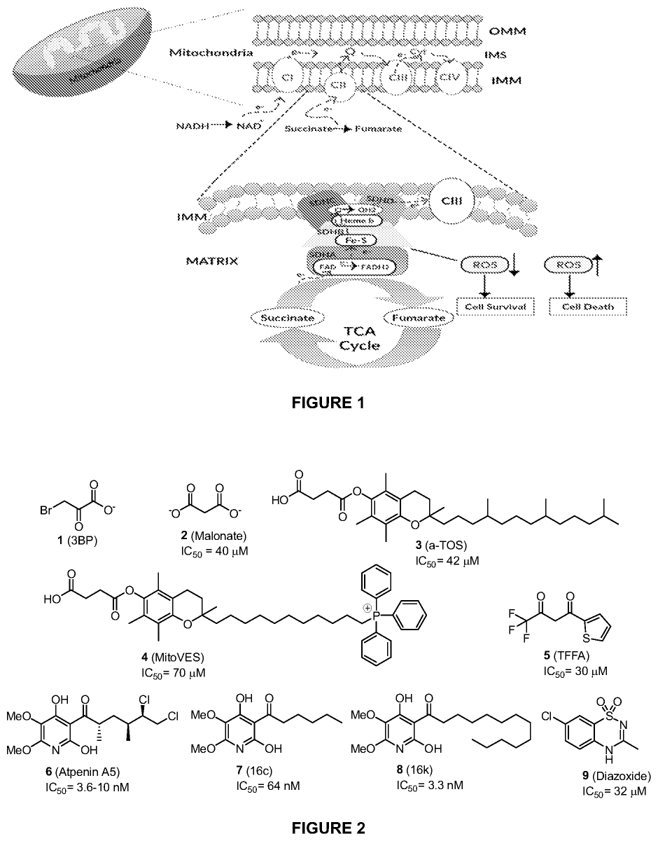

Mitochondrial respiratory complex II (CII), or succinate dehydrogenase (SDH), is a well-characterized 124 kDa protein complex located to the inner membrane of mitochondria (FIG. 1). Recently, it has attracted considerable attention as a therapeutic target. The protein plays a vital role in mitochondrial metabolism, where it catalyzes the oxidation of succinate to fumarate and the reduction of ubiquinone (UQ) to ubiquinol (UQH2). Mitochondrial complex II connects the tricarboxylic acid cycle (TCA) and the electron transport chain (ETC), while lacking any contribution to maintaining the proton gradient across the mitochondrial inner membrane in comparison to other complexes. Inhibition of the ETC induces apoptosis through the generation of reactive oxygen species (ROS).

Many factors make CII an emerging therapeutic target for several human diseases, including myocardial infarction, stroke, and cancer. Mitochondrial complex II has a significant promise for the development of selective small molecule chemotherapeutics. In addition to the generation of ROS by inhibition of CII, glutaminolysis, the primary source of energy for cancer cells, is also impaired through inhibition of the TCA cycle. Mitochondrial complex II is a vital member of the NADH-fumarate reductase system and is involved in the maintenance of mitochondrial energy production in tumor microenvironments under hypoxic conditions. The inhibition of CII leads to the activation of both autophagy and apoptosis in tumor cells, which could be an appropriate strategy for combating drug resistance. Promisingly, known CII inhibitors are selectively cytotoxic to cancer cells possessing negligible effects on healthy cells. Mutation of CII is rare, which makes it a unique target for drug development. Such mutations are associated only in infrequent and nonaggressive neoplasias such as pheochromocytomas.

Most reported CII inhibitors in the literature (FIG. 2) exhibit only moderately potent CII inhibition. The alkylating agent and hexokinase inhibitor 3-Bromopyruvate (1, 3BP), was the first identified CII inhibitor, but no IC₅₀ has been reported. Malonate (2) was one of the first identified CII inhibitors with an IC₅₀ value of 40 μM, and is often used as a reference compound. The vitamin E analog α-Tocopheryl succinate (3, α-TOS) has a CII IC₅₀ value of 42 μM, and is known to induce apoptosis in cancer cells by ROS generation. Mitochondrially targeted vitamin E succinate (4, MitoVES) has an CII IC₅₀ of 70 μM, although it was 20-50 times more effective in inducing apoptosis in cancer cells than 3. This is attributed to the introduction of a cationic triphenylphosphonium (TPP) group which acts to target the compound to mitochondria. The inhibition of CII has been shown to be selective to cancerous cells with MitoVES possessing an IC₅₀ of 0.5-3 μM for apoptosis induction in cancer cells and approximately 20-60 μM for non-malignant cells.²⁷ Thenoyltrifluoroacetone (5, TFFA), with an IC₅₀ value of 30 μM is widely used as a control compound in CII assay kits. The natural product atpenin A5 (6, AA5) is a potent and specific CII inhibitor at the ubiquinone binding site (IIQ), with IC₅₀ of 3.6-10 nM. Atpenin A5 derivative 16c (7) has been previously reported, which possess an IC₅₀ value of 64 nM and a ‘drug-like’ ligand-lipophilicity efficiency of 5.62. Compound 7 was shown to induce the anti-proliferative activity in multiple prostate cancer cells. Furthermore, Atpenin A5 derivative 16 k (8) with an IC₅₀ of 3.3 nM, was reported from the same study as the most potent CII inhibitor described to date, albeit with limiting lipophilicity.

The Food and Drug Administration (FDA)-approved clinical vasodilator drug diazoxide (9, DZX) has a CII IC₅₀ value of 32 μM in rat heart mitochondria, and is known to regulate ROS production, protecting normal cells from ischemic damage and also inducing specific cancer cell death. Further reports found that high doses of diazoxide (750 μM) led to inhibition of CII and increase in ROS levels, while concentrations of diazoxide <200 μM had no effect to depolarize the mitochondrial membrane of cortical neurons, but a 300 μM dose did result in depolarization. Further, a 100 μM concentration of diazoxide was reported to inhibit mouse heart mitochondrial CII but IC₅₀ was not reached. Diazoxide has been shown to be neuroprotective in animal models of Alzheimer's disease, protect neurons from a range of neurotoxic insults, including exposure to amyloid-β peptide (25-35), and was reported to reduce proliferation in acute leukemic T cells, and triple negative breast cancer (TNBC) MDA-MB 468 cells. One mechanism of action of this observed cytotoxicity was attributed to the downregulation of beta-catenin-mediated Cyclin D1 transcription. No studies have been reported that attempt to probe the structure-activity relationship (SAR) of diazoxide for CII inhibition or antineoplastic activity.

A library of novel diazoxide derivatives are disclosed herein and was used to understand structural effect on CII inhibition activity. The library was further used to evaluate and demonstrate the antineoplastic effect in 22Rv1 prostate cancer cells and the TNBC MDA-MB-468 cell line. The diazoxide parent compound exhibited no CII inhibition activity at 100 μM (IC₅₀ was found to be 1236 μM) which corresponded to no effect to reduce cell viability of either prostate or breast cancer cells at 100 μM up to 72 hours. Several derivatives were identified that possessed enhanced (but still moderate) activity to inhibit CII (IC₅₀ values 11.88-89 μM) over diazoxide which resulted in increased effect to reduce cell viability in the breast cancer but not prostate cancer cell lines. Importantly, eight diazoxide derivatives were identified that possessed potent activity to reduce TNBC cell viability which represent novel hit compounds for further optimization as potential therapeutics for this aggressive and difficult to treat cancer.

### VEGF Inhibitors

Antiangiogenic drugs that inhibit the action of VEGF have been shown to normalize tumor vasculature and, as a result, offer an improvement in chemotherapeutic delivery. Anti-VEGF drugs are often effective in clinical practice when combined with other chemotherapeutic anticancer agents. Anti-VEGF treatments are designed to target both the pro-angiogenic activity and the antiapoptotic functions of VEGF. As used herein, the term “VEGF inhibitor” refers to a compound or composition that inhibits the action of VEGF, and the terms “VEGF inhibition” or “inhibiting VEGF” or their equivalents refer to inhibiting the action or binding of a VEGF to a VEGF receptor (“VEGFR”).

It is known that vascular endothelial growth factor (VEGF) plays a pivotal role in the growth of the abnormal blood vessels (i.e. choroidal neovascularization, CNV) which plays a role in the development of eye diseases and disorders. For example, age-related macular degeneration (AMD) is a leading cause of blindness among elderly patients in developed countries, and the “wet form” of AMD is known to be linked to VEGF-related abnormal vascularization. See e.g., Current drug targets 12.2 (2011): 173-181 The role of VEGF is also being investigated in other eye diseases and disorders, such as retinal vascular disease, coats disease, submacular hemorrhage, wet macular degeneration, and neovascular age-related degeneration. See e.g., clinical trial nos. NCT03940690 and NCT03699618, available on clinicaltrials.gov.

Halogenated benzothiadiazines based on the structure of the clinical vasodilator and FDA approved drug diazoxide (DZX) have been shown to have antineoplastic activity. DZX inhibited VEGF-mediated angiogenesis has been reported in an in vivo Matrigel plug assay in mice. Without wishing to be bound by theory, it is thought that a guanidine-like template comprises the pharmacophore of many VEGF (and other kinase) inhibitors. Without wishing to be bound by theory, it is thought that the guanidine unit occupies the hydrophilic site within the hydrophobic pocket of VEGF2 and forms a strong hydrogen bond with the Asp331 amino acid residue. The clinically approved VEGF inhibitor drugs sorafenib, regorafenib, lenvatinib, and others possess a urea moiety, a bioisostere of the guanidine group, which contributes to the biological activity of these compounds. A recent study reported the sulfonylurea unit as a suitable bioisotere for the urea, which retained VEGF inhibition activity. Sulfonamide derivatives are known to have many biological activities, including antimicrobial, antiinflammatory, and anticancer activity. Without wishing to be bound by theory, it is believed that terminal electron withdrawing groups of the benzothiadiazines reported herein are beneficial for stability and activity.

Novel antiangiogenesis agents described herein comprise cyclic sulfonamide derivatives of the benzothiadiazine class. Provided herein are syntheses and data regarding the antineoplastic effect and VEGF inhibition activity of these benzothiadiazine derivatives. Benzothiadiazine derivatives were screened for their ability to inhibit the VEGF-induced proliferation of human umbilical vein endothelial cells (HUVECs). Compounds 18b, 20b, 21b, 22b, 23b, 29b, 30b, 41b, 42b, 43b, 56e, 57e, 58e, 59e, 60e, 61e, 62e, 63e, 64e, 65e, and 66e suppressed HUVEC proliferation and selected derivatives significantly reduced phosphorylation of vascular endothelial growth factor receptor 2 (VEGFR2).

### Compounds of the Disclosure

The present disclosure describes compounds, compositions, and methods for the treatment of cancer. The compounds and compositions of the present disclosure comprise halogenated benzothiadiazines. The disclosure describes improved or complementary chemotherapeutics for any number of cancers including prostate cancer and triple negative breast cancer.

Inhibition of metabolomic pathways are a common approach for deriving chemotherapies as they help overcome drug resistance, enhance the specificity of cancer cell targeting and increase the potency of existing treatments. The Krebs cycle involves the oxidation of acetyl-CoA which yields ATP and is commonly exploited by cancer cells to over produce energy. Within this system is Mitochondrial respiratory complex II which when inhibited impairs glutaminolysis and can activate autophagy, all promising phenotypes for the treatment of cancer.

Mitochondrial respiratory complex II (CII), or succinate dehydrogenase (SDH), is a well-characterized 124 kDa protein complex located to the inner membrane of mitochondria. The protein plays a vital role in mitochondrial metabolism, where it catalyzes the oxidation of succinate to fumarate and the reduction of ubiquinone (UQ) to ubiquinol (UQH2). Mitochondrial complex II connects the tricarboxylic acid cycle (TCA) and the electron transport chain (ETC), while lacking any contribution to maintaining the proton gradient across the mitochondrial inner membrane in comparison to other complexes. Inhibition of the ETC induces apoptosis through the generation of reactive oxygen species (ROS). Compounds and compositions of the present disclosure can inhibit the Mitochondrial respiratory complex II.

Generic inhibitors of this complex available which are used for treating hypoglycemia. There is evidence these inhibitors could be effective in highly drug resistant and difficult to target cancers like castration resistant prostate cancer and triple negative breast cancer.

The compositions of the present disclosure can be used to treat and/or prevent a variety of diseases and disorders. In one embodiment, the disease or disorder is a disease for which inhibition of the mitochondrial respiratory complex II (CII) or the vascular endothelial growth factor (VEGF) pathway is useful.

In one embodiment the disease or disorder is a form of cancer. In one embodiment the compositions of the present disclosure can be administered with at least one other therapeutic agent (e.g. other anti-cancer agents).

In one embodiment the disease or disorder is a neurodegenerative disease including but not limited to Alzheimer's disease, Parkinson's disease, motor neuron disease, and spinal muscular atrophy. In one embodiment the disease or disorder is stroke and complications associated with stroke.

In one embodiment, the disease or disorder is an eye disease or disorder.

The compositions of the present disclosure may be administered by any method. Methods of administration include but are not limited to parenterally, subcutaneously, orally, topically, pulmonarily, rectally, vaginally, intravenously, intraperitoneally, intrathecally, intracerbrally, epidurally, intramuscularly, intradermally, or intracarotidly.

The disclosure provides compounds of Formula I:

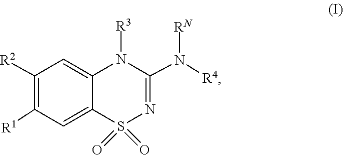

wherein

R^{N} is H or C₁₋₆ alkyl;

R¹ and R² are each independently H, halo, CN, C₁₋₆ haloalkyl, isothiocyanate, or —OSO₂R^{S}, wherein R^{S} is C₁₋₃ alkyl, C₁₋₃ haloalkyl, or phenyl optionally substituted with one of halo, C₁₋₃ alkyl, or NO₂;

R³ is H, C₁₋₆ alkyl, C₃₋₁₀ cycloalkyl, or C₆₋₁₀ aryl;

R⁴ is C₀₋₆alkylene-C₃₋₆ cycloalkyl, C₀₋₆alkylene-3-12 membered heterocycloalkyl having 1-3 ring heteroatoms selected from O, S, and N, C₀₋₆alkylene-C₆₋₁₀ aryl, or C₀₋₆alkylene-5-10 membered heteroaryl having 1-4 heteroatoms selected from N, O, and S, and said cycloalkyl, heterocycloalkyl, aryl, and heteroaryl are optionally substituted with one or more R⁵; and

each R⁵ is independently halo, C₁₋₆ alkyl, C₁₋₆ haloalkyl, C₁₋₆ alkoxy, C₁₋₆ haloalkoxy, S—C₁₋₆ haloalkyl, or SO₂N(R^{N})₂,

with the proviso that the compound is not:
3-(cyclopentylamino)-7-fluoro-4H-benzo[e][1,2,4]thiadiazine 1,1-dioxide;
6-chloro-3-(cyclopentylamino)-4H-benzo[e][1,2,4]thiadiazine 1,1-dioxide; or
6-chloro-3-(phenylamino)-4H-benzo[e][1,2,4]thiadiazine 1,1-dioxide.

In some cases, R^{N} is H. In some cases, R^{N} is C₁₋₆ alkyl. In some cases, R^{N} is methyl.

In some cases, R³ is H or C₁₋₆ alkyl. In some cases, R³ is H. In some cases, R³ is C₃₋₁₀ cycloalkyl or C₆₋₁₀ aryl.

In some cases, R¹ is H, halo, or CN. In some cases, R¹ is H, F, Cl, Br, or CN. In some cases, R¹ is H, F, Br, or CN. In some cases, In some cases, R¹ is H or halo. In some cases, R¹ is H, F, or Br. In some cases, R¹ is H or Cl. In some cases, R¹ is H. In some cases, R¹ is F or Cl. In some cases, R¹ is F or Br. In some cases, R¹ is Cl or Br. In some cases, R¹ is F. In some cases, R¹ is Cl. In some cases, R¹ is Br.

In some cases, R² is H, halo, or CN. In some cases, R² is H, Cl, or CN. In some cases, R² is H or halo. In some cases, R² is H or Cl. In some cases, R² is H. In some cases, R² is Cl. In some cases, R¹ is H, F, or Br and R² is H or Cl. In some cases, R¹ is F and R² is H.

In some cases, R¹ is Cl and R² is H. In some cases, R¹ is H and R² is H. In some cases, R¹ is H and R² is Cl.

In some cases, R⁴ is C₀alkylene-C₃₋₆ cycloalkyl or C₀alkylene-C₆₋₁₀ aryl. In some cases, R⁴ is cyclopentyl, phenyl, or naphthyl. In some cases, R⁴ is phenyl. In some cases, R⁴ is cyclopentyl. In some cases, R⁴ is C₁₋₆alkylene-C₃₋₆ cycloalkyl, C₁₋₆alkylene-3-12 membered heterocycloalkyl having 1-3 ring heteroatoms selected from O, S, and N, C₁₋₆alkylene-C₆₋₁₀ aryl, or C₁₋₆alkylene-5-10 membered heteroaryl having 1-4 heteroatoms selected from N, O, and S. In some cases, R⁴ is C₁₋₆alkylene-C₆₋₁₀ aryl or C₁₋₆alkylene-5-10 membered heteroaryl. In some cases, R⁴ is C₁₋₆alkylene-phenyl, C₁₋₆alkylene-pyridinyl, or C₁₋₆alkylene-indolyl. In some cases, R⁴ is C₁₋₆alkylene-phenyl or C₁₋₆alkylene-indolyl. In some cases, R⁴ is C₁₋₄₆alkylene-phenyl, C₁₋₄alkylene-pyridinyl, or C₁₋₄alkylene-indolyl. In some cases, R⁴ is C₁₋₄alkylene-phenyl or C₁₋₄alkylene-indolyl. In some cases, R⁴ is

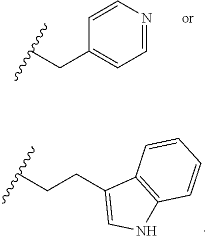 [SMILES: *Cc1ccncc1 ; *CCc1c[nH]c2ccccc12]

In some cases, R⁴ is

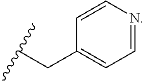 [SMILES: *Cc1ccncc1]

In some cases, R⁴ is

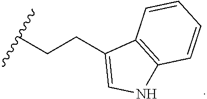 [SMILES: *CCc1c[nH]c2ccccc12]

In some cases, R⁴ is C₁₋₆alkylene-phenyl. In some cases, R⁴ is C₁₋₄alkylene-phenyl. In some cases, R⁴ is benzyl. In some cases, R⁴ is

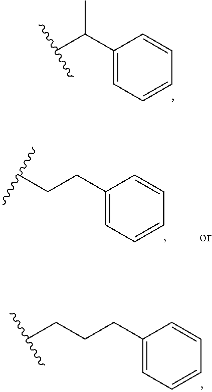

In some cases, R⁴ is

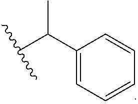 [SMILES: *C(C)c1ccccc1]

In some cases, R⁴ is

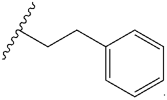 [SMILES: *CCc1ccccc1]

In some cases, R⁴ is

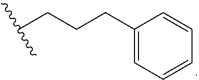 [SMILES: *CCCc1ccccc1]

In some cases, R⁴ is unsubstituted. In some cases, R⁴ is substituted with one or more R⁵. In some cases, R⁴ is substituted with one R⁵. In some cases, R⁴ is substituted with two R⁵. In some cases, R⁴ is substituted with three R⁵. In some cases, each R⁵ is halo, C₁₋₆ alkyl, C₁₋₆ haloalkyl, C₁₋₆ alkoxy, or C₁₋₆ haloalkoxy. In some cases, each R⁵ is halo. In some cases, each R⁵ is C₁₋₆ alkyl. In some cases, each R⁵ is F, Cl, methyl, trifluoromethyl, methoxy, or trifluoromethoxy. In some cases, each R⁵ is F. In some cases, each R⁵ is C₁₋₆ alkoxy or C₁₋₆ haloalkoxy. In some cases, each R⁵ is methoxy or trifluoromethoxy.

In some cases, R⁴ is C₁₋₄alkylene-C₆₋₁₀ aryl substituted with one or more R⁵. In some cases, R⁴ is C₁₋₄alkylene-C₆₋₁₀ aryl substituted with one R⁵. In some cases, R⁴ is C₁₋₄alkylene-C₆₋₁₀ aryl substituted with two R⁵. In some cases, R⁴ is C₁₋₄alkylene-C₆₋₁₀ aryl substituted with three R⁵. In some cases, R⁴ is C₁₋₄alkylene-phenyl substituted with one or more R⁵. In some cases, R⁴ is C₁₋₄alkylene-phenyl substituted with one R⁵. In some cases, R⁴ is C₁₋₄alkylene-phenyl substituted with two R⁵. In some cases, R⁴ is C₁₋₄alkylene-phenyl substituted with three R⁵. In some cases, R⁴ is

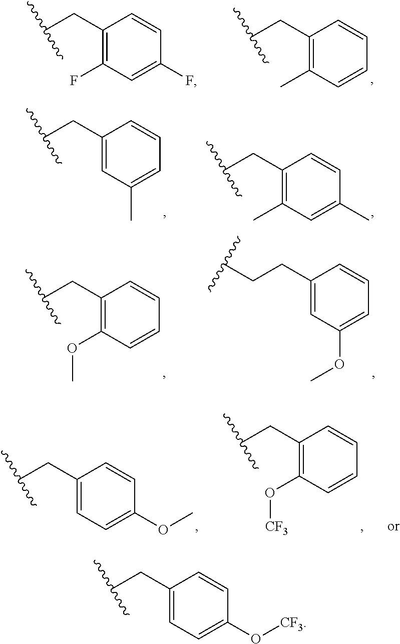

In some cases, R⁴ is

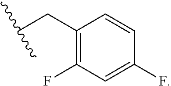 [SMILES: *Cc1ccc(F)cc1F]

In some cases, R⁴ is

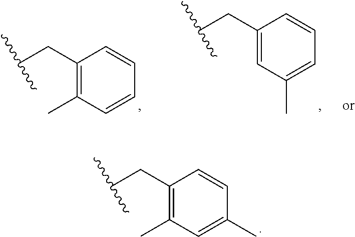

In some cases, R⁴ is

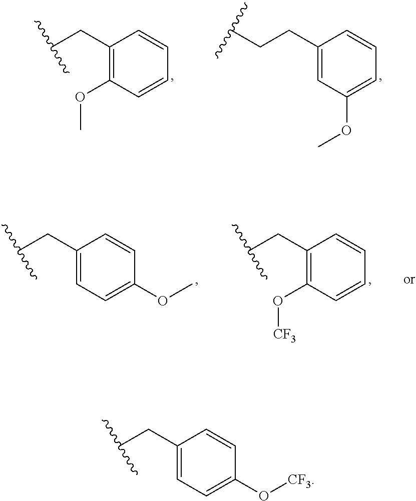

In some cases, R⁴ is

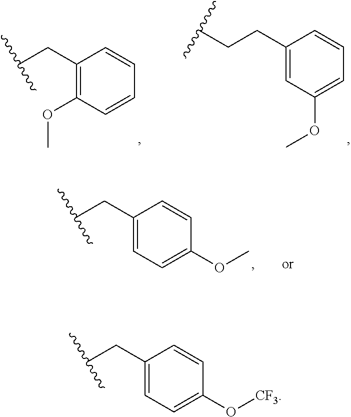

In some cases, R⁴ is

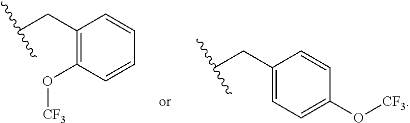 [SMILES: *Cc1ccccc1OC(F)(F)F ; *Cc1ccc(OC(F)(F)F)cc1]

In some cases, R⁴ is

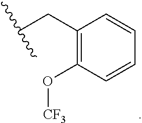 [SMILES: *Cc1ccccc1OC(F)(F)F]

In some cases, R⁴ is

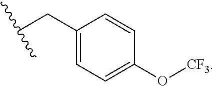 [SMILES: *Cc1ccc(OC(F)(F)F)cc1]

Specific compounds contemplated include those listed in Table 1, or a pharmaceutically acceptable salt thereof:

TABLE 1

| | Structure | | |
|---|---|---|---|
| | 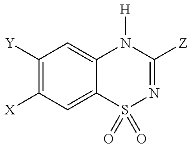 | | |
| Compound | X | Y | Z |
| 9a | F | H | 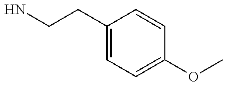 |
| 16a | F | H | 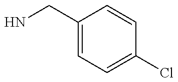 |
| 17a | F | H | 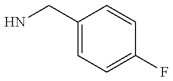 |
| 18a | F | H | 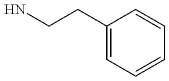 |
| 20a | F | H | 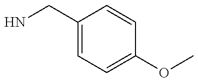 |
| 21a | F | H | 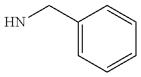 |
| 23a | F | H | 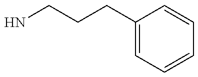 |
| 24a | F | H | 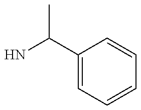 |
| 25a | F | H | 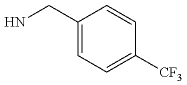 |
| 26a | F | H | 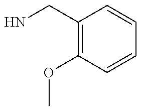 |
| 27a | F | H | 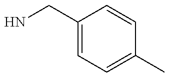 |
| 30a | F | H | 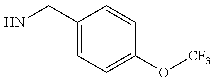 |
| 36a | F | H | 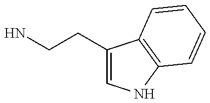 |
| 37a | F | H | 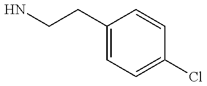 |
| 38a | F | H | 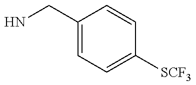 |
| 39a | F | H | 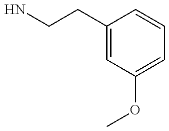 |
| 41a | F | H | 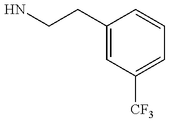 |
| 43a | F | H | 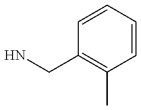 |
| 44a | F | H | 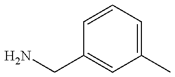 |
| 45a | F | H | 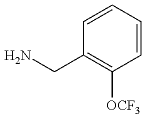 |
| 46a | F | H | 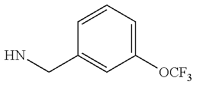 |
| 47a | F | H | 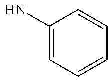 |
| 16b | H | Cl | 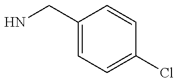 |
| 17b | H | Cl | 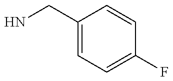 |
| 18b | H | Cl | 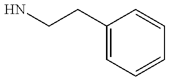 |
| 20b | H | Cl | 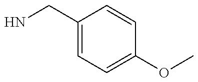 |
| 21b | H | Cl | 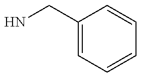 |
| 23b | H | Cl | 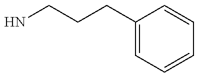 |
| 24b | H | Cl | 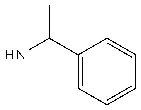 |
| 25b | H | Cl | 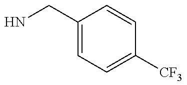 |
| 26b | H | Cl | 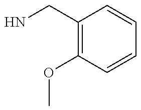 |
| 27b | H | Cl | 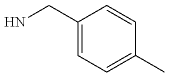 |
| 28b | H | Cl | 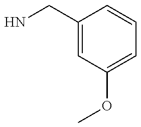 |
| 29b | H | Cl | 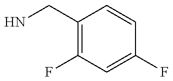 |
| 30b | H | Cl | 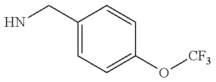 |
| 32b | H | Cl | 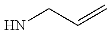 |
| 36b | H | Cl | 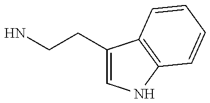 |
| 40b | H | Cl | 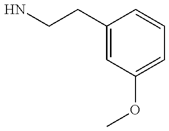 |
| 41b | H | Cl | 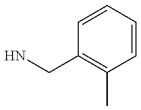 |
| 42b | H | Cl | 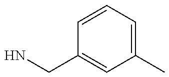 |
| 43b | H | Cl | 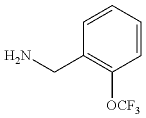 |
| 44b | H | Cl | 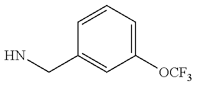 |
| 16c | Br | H | 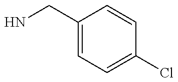 |
| 17c | Br | H | 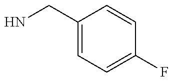 |
| 18c | Br | H | 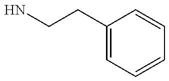 |
| 20c | Br | H | 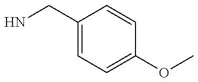 |
| 21c | Br | H | 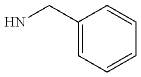 |
| 22c | Br | H | 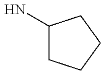 |
| 23c | Br | H | 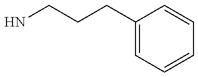 |
| 24c | Br | H | 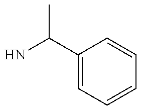 |
| 25c | Br | H | 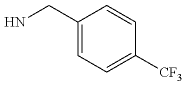 |
| 26c | Br | H | 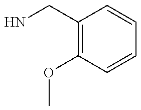 |
| 27c | Br | H | 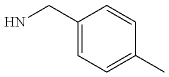 |
| 28c | Br | H | 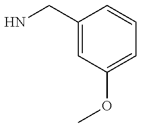 |
| 29c | Br | H | 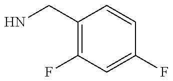 |
| 30c | Br | H | 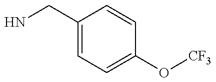 |
| 31c | Br | H | 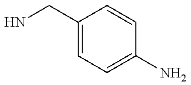 |
| 33c | Br | H | 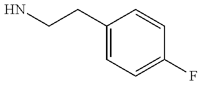 |
| 34c | Br | H | 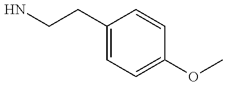 |
| 35c | Br | H | 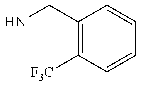 |
| 36c | Br | H | 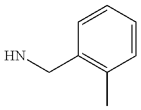 |
| 37c | Br | H | 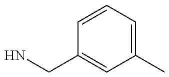 |
| 38c | Br | H | 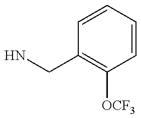 |
| 39c | Br | H | 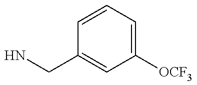 |
| 40c | Br | H | 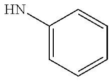 |
| 15d | H | H | 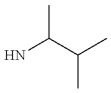 |
| 16d | H | H | 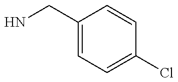 |
| 17d | H | H | 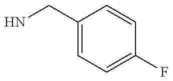 |
| 20d | H | H | 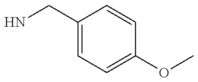 |
| 21d | H | H | 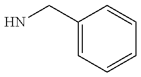 |
| 23d | H | H | 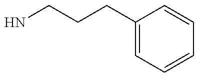 |
| 24d | H | H | 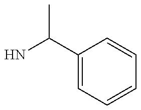 |
| 30d | H | H | 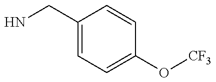 |
| 32d | H | H | 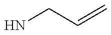 |
| 44d | H | H | 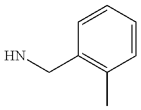 |
| 45d | H | H | 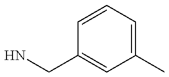 |
| 46d | H | H | 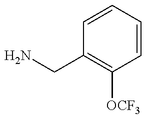 |
| 47d | H | H | 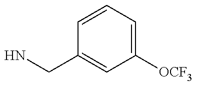 |
| 48d | H | H | 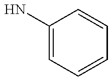 |
| 54e | Cl | H | 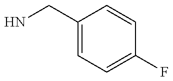 |
| 55e | Cl | H | 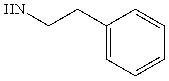 |
| 56e | Cl | H | 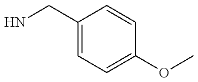 |
| 57e | Cl | H | 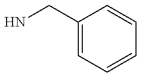 |
| 58e | Cl | H | 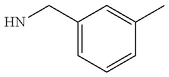 |
| 59e | Cl | H | 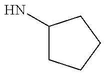 |
| 60e | Cl | H | 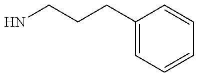 |
| 61e | Cl | H | 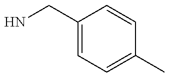 |
| 62e | Cl | H | 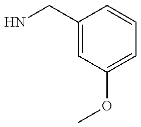 |
| 63e | Cl | H | 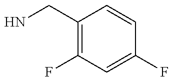 |
| 64e | Cl | H | 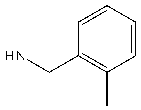 |
| 65e | Cl | H | 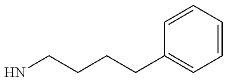 |
| 66e | Cl | H | 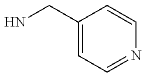 |
| 67e | Cl | H | 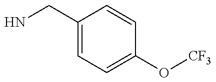 |
| 68e | Cl | H | 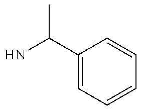 |
| 16e | H | CN | 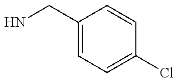 |
| 17e | H | CN | 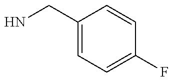 |
| 18e | H | CN | 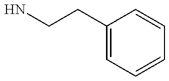 |
| 20e | H | CN | 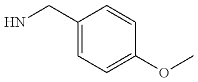 |
| 21e | H | CN | 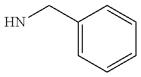 |
| 22e | H | CN | 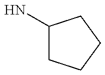 |
| 23e | H | CN | 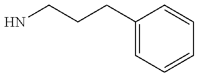 |
| 24e | H | CN | 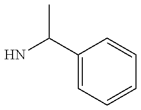 |
| 25e | H | CN | 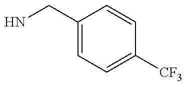 |
| 26e | H | CN | 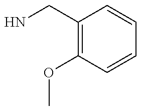 |
| 27e | H | CN | 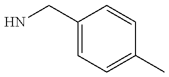 |
| 28e | H | CN | 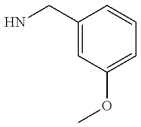 |
| 29e | H | CN | 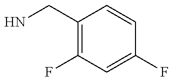 |
| 30e | H | CN | 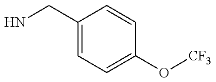 |
| 32e | H | CN | 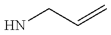 |
| 36e | H | CN | 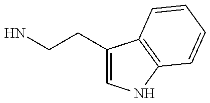 |
| 40e | H | CN | 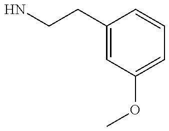 |
| 41e | H | CN | 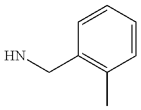 |
| 42e | H | CN | 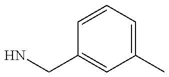 |
| 43e | H | CN | 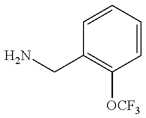 |
| 44e | H | CN | 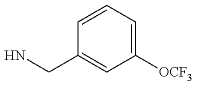 |
| 45e | H | CN | 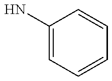 |
| 13f | CN | H | SCH₃ |
| 15f | CN | H | 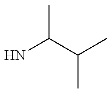 |
| 16f | CN | H | 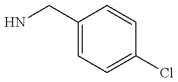 |
| 17f | CN | H | 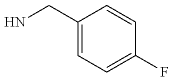 |
| 18f | CN | H | 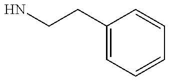 |
| 19f | CN | H | NHCH₂CH₃ |
| 20f | CN | H | 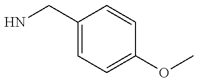 |
| 21f | CN | H | 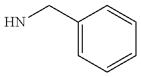 |
| 22f | CN | H | 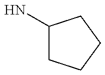 |
| 23f | CN | H | 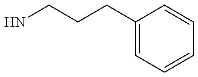 |
| 24f | CN | H | 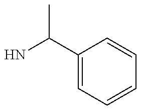 |
| 25f | CN | H | 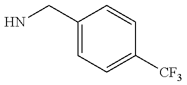 |
| 26f | CN | H | 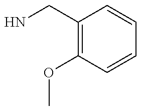 |
| 27f | CN | H | 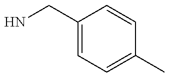 |
| 28f | CN | H | 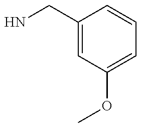 |
| 29f | CN | H | 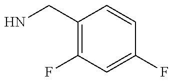 |
| 30f | CN | H | 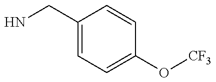 |
| 32f | CN | H | 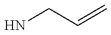 |
| 36f | CN | H | 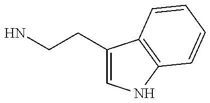 |
| 40f | CN | H | 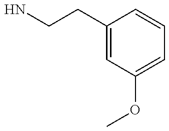 |
| 41f | CN | H | 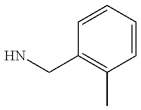 |
| 42f | CN | H | 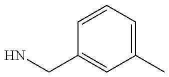 |
| 43f | CN | H | 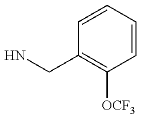 |
| 44f | CN | H | 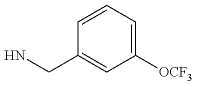 |
| 45f | CN | H | 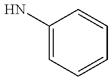 |
| | 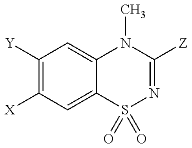 | | |
| | X | Y | Z |
| 42a | F | H | SCH₃ |
| 42d | H | H | SCH₃ |
| 43d | H | H | 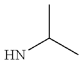 |
| | 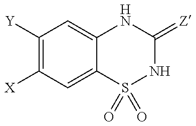 | | |
| | X | Y | Z′ |
| 11a | F | H | O |
| 12a | F | H | S |
| 11b | H | Cl | O |
| 12b | H | Cl | S |
| 11c | Br | H | O |
| 12c | Br | H | S |
| 11d | H | H | O |
| 12d | H | H | S |

Additional specific compounds contemplated include those listed in Table 2, or a pharmaceutically acceptable salt thereof:

TABLE 2

| | Structure | | |
|---|---|---|---|
| | 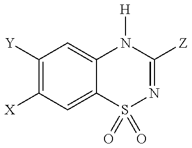 | | |
| Compound | X | Y | Z |
| 13a | F | H | SCH₃ |
| 14a | F | H | 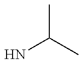 |
| 15a | F | H | 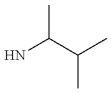 |
| 19a | F | H | NHCH₂CH₃ |
| 22a | F | H | 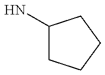 |
| 32a | F | H | 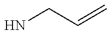 |
| 42a | F | H | SCH₃ |
| 13b | H | Cl | SCH₃ |
| 14b | H | Cl | 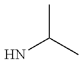 |
| 15b | H | Cl | 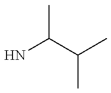 |
| 19b | H | Cl | NHCH₂CH₃ |
| 22b | H | Cl | 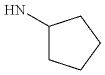 |
| 45b | H | Cl | 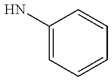 |
| 13c | Br | H | SCH₃ |
| 14c | Br | H | 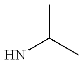 |
| 15c | Br | H | 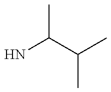 |
| 19c | Br | H | NHCH₂CH₃ |
| 13d | H | H | SCH₃ |
| 14d | H | H | 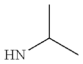 |
| 19d | H | H | NHCH₂CH₃ |
| 13e | H | CN | SCH₃ |
| 14e | H | CN | 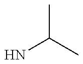 |
| 15e | H | CN | 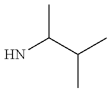 |
| 19e | H | CN | NHCH₂CH₃ |
| 14f | CN | H | 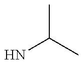 |
| 53e | Cl | H | SCH₃ |
| | | | 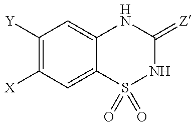 |
| | X | Y | Z′ |
| 11a | F | H | O |
| 12a | F | H | S |
| 11c | Br | H | O |
| 12c | Br | H | S |
| 51e | Cl | H | O |
| 52e | Cl | H | S |

In some cases, the compound is selected from 18c, 20c, 23a, 24a, 24b, 24c, 24d, 26a, 30a, 30b, 30c, 30d, 36a, and 39a. In some cases, the compound is selected from 18b, 20b, 21b, 22b, 23b, 29b, 30b, 41b, 42b, 43b, 24d, 56e, 57e, 58e, 59e, 60e, 61e, 62e, 63e, 64e, 65e, and 66e. In some cases, the compound is selected from 18b, 20b, 21b, 22b, 23b, 29b, 30b, 41b, 42b, 43b, 56e, 57e, 58e, 59e, 60e, 61e, 62e, 63e, 64e, 65e, and 66e. In some cases, the compound is selected from 30b, 59e, 62e, and 24d. In some cases, the compound is selected from 21 b, 22b, 29b, 30b, 43b, 58e, 59e, 62e, 30c, 67e, 30d, 24b, 24c, 68e, and 24d.

The compounds disclosed herein can be in the form of a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This disclosure also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Definitions

As used herein, the term “alkyl” refers to straight chained and branched saturated hydrocarbon groups containing one to six carbon atoms. The term Cₙ means the alkyl group has “n” carbon atoms. For example, C₆ alkyl refers to an alkyl group that has 6 carbon atoms. C₁-C₆ alkyl refers to an alkyl group having a number of carbon atoms encompassing the entire range (e.g., 1 to 6 carbon atoms), as well as all subgroups (e.g., 1-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group.

As used herein, the term “alkoxy” refers to a “—O-alkyl” group. The alkoxy group can be unsubstituted or substituted.

The term “alkylene” used herein refers to an alkyl group having a substituent. For example, an alkylene group can be —CH₂CH₂— or —CH₂—. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups. The term “C₀alkylene” means a bond. Unless otherwise indicated, an alkylene group can be an unsubstituted alkylene group or a substituted alkylene group.

As used herein, the term “haloalkyl” refers to an alkyl group substituted with one or more halogen substituents. For example, C₁-C₆haloalkyl refers to a C₁-C₆ alkyl group substituted with one or more halogen atoms, e.g., 1, 2, 3, 4, 5, or 6 halogen atoms. Non-limiting examples of haloalkyl groups include fluoromethyl, difluoromethyl, trifluoromethyl, chloromethyl, dichloromethyl, and trichloromethyl groups. Similarly, haloalkoxy refers to an alkoxy group substituted with one or more halogen atoms e.g., 1, 2, 3, 4, 5, or 6 halogen atoms.

As used herein, the term “halo” or “halogen” refers to fluorine, chlorine, bromine, or iodine.

As used herein, the term “isothiocyanate” refers to a —N═C═S group.

As used herein, the term “cycloalkyl” refers to an aliphatic cyclic hydrocarbon group containing three to ten carbon atoms (e.g., 3, 4, 5, 6, 7, 8, 9, or 10 carbon atoms). The term Cₙ means the cycloalkyl group has “n” carbon atoms. For example, C₅ cycloalkyl refers to a cycloalkyl group that has 5 carbon atoms in the ring. C₃-C₁₀ cycloalkyl refers to cycloalkyl groups having a number of carbon atoms encompassing the entire range (e.g., 3 to 10 carbon atoms), as well as all subgroups (e.g., 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 7-8, 6-9, 6-10, 6, 7, 8, 9, and 10 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group.

As used herein, the term “heterocycloalkyl” is defined similarly as cycloalkyl, except the ring contains one to three heteroatoms independently selected from oxygen, nitrogen, and sulfur. In particular, the term “heterocycloalkyl” refers to a ring containing a total of three to twelve atoms (e.g., three to seven, or five to ten), of which 1, 2, 3 or three of those atoms are heteroatoms independently selected from the group consisting of oxygen, nitrogen, and sulfur, and the remaining atoms in the ring are carbon atoms. Nonlimiting examples of heterocycloalkyl groups include piperdine, pyrazolidine, tetrahydrofuran, tetrahydropyran, dihydrofuran, morpholine, and the like.

As used herein, the term “aryl” refers to a monocyclic aromatic group, such as phenyl. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Other substituents are also contemplated, including C₀₋₃ alkylene-halo, C₀₋₃ alkylene-CN, C₀₋₃ alkylene-NH₂, C₀₋₃ alkylene-OH, and C₀₋₃ alkylene-O—C₁₋₃alkyl. Aryl groups can be isolated (e.g., phenyl) or fused to another aryl group (e.g., naphthyl, anthracenyl), a cycloalkyl group (e.g. tetraydronaphthyl), a heterocycloalkyl group, and/or a heteroaryl group. Exemplary aryl groups include, but are not limited to, phenyl, chlorophenyl, methylphenyl, methoxyphenyl, trifluoromethylphenyl, nitrophenyl, 2,4-methoxychlorophenyl, and the like. Throughout, the abbreviation “Ph” refers to phenyl and “Bn” refers to benzyl (i.e., CH₂phenyl).

As used herein, the term “heteroaryl” refers to an aromatic ring having 5 to 10 total ring atoms, and containing one to four heteroatoms selected from nitrogen, oxygen, and sulfur atom in the aromatic ring. Unless otherwise indicated, a heteroaryl group can be unsubstituted or substituted with one or more, and in particular one to four, substituents Examples of heteroaryl groups include, but are not limited to, thienyl, furyl, pyridyl, pyrrolyl, oxazolyl, triazinyl, triazolyl, isothiazolyl, isoxazolyl, indolyl, imidazolyl, pyrazinyl, pyrimidinyl, thiazolyl, and thiadiazolyl.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

As used herein, the phrase “optionally substituted” means unsubstituted (e.g., substituted with a H) or substituted. As used herein, the term “substituted” means that a hydrogen atom is removed and replaced by a substituent. It is understood that substitution at a given atom is limited by valency. The use of a substituent (radical) prefix name such as alkyl without the modifier “optionally substituted” or “substituted” is understood to mean that the particular substituent is unsubstituted.

As used herein, the term “therapeutically effective amount” means an amount of a compound or combination of therapeutically active compounds (e.g., a mitochondrial respiratory complex II (CII) or vascular endothelial growth factor (VEGF) inhibitor or combination of mitochondrial respiratory complex II (CII) or vascular endothelial growth factor (VEGF) inhibitors) that ameliorates, attenuates or eliminates one or more symptoms of a particular disease or condition (e.g., cancer), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans). The terms patient and subject include males and females.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

As used herein the terms “treating”, “treat” or “treatment” and the like include preventative (e.g., prophylactic) and palliative treatment.

As used herein, the term “excipient” means any pharmaceutically acceptable additive, carrier, diluent, adjuvant, or other ingredient, other than the active pharmaceutical ingredient (API).

### Synthesis of Compounds of the Disclosure

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, are useful and recognized reference textbooks of organic synthesis known to those in the art. For example, the compounds disclosed herein can be synthesized by solid phase synthesis techniques including those described in Merrifield, J. Am. Chem. Soc. 1963; 85:2149; Davis et al., Biochem. Intl. 1985; 10:394-414; Larsen et al., J. Am. Chem. Soc. 1993; 115:6247; Smith et al., J. Peptide Protein Res. 1994; 44: 183; O'Donnell et al., J. Am. Chem. Soc. 1996; 118:6070; Stewart and Young, Solid Phase Peptide Synthesis, Freeman (1969); Finn et al., The Proteins, 3rd ed., vol. 2, pp. 105-253 (1976); and Erickson et al., The Proteins, 3rd ed., vol. 2, pp. 257-527 (1976). The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt, ester or prodrug thereof.

The parent compound diazoxide (7-chloro-3-methyl-2H-1,2,4-benzothiadiazine 1,1-dioxide) (9) can be accessed by a number of syntheses. Additionally, a number of chain derivatives of 9 have been synthesized as K_{ATP} channel activators that are selective to pancreatic B-cells, although no determination of antineoplastic effects of these compounds have been reported. Halogen substituted Diazoxide analogs at the 4- and or 5-position of the phenyl ring can be accessed over four steps (Scheme 1) starting from an appropriately substituted aniline (10a-e). Electrophilic substitution of the appropriate aniline with chlorosulfonyl isocyanate in the presence of anhydrous aluminum chloride and nitromethane results in ring closure to yield 6 and 7 halo-3-oxo-3,4-dihydro-2H-1,2,4-benzothiadiazine1,1dioxides (11a-c, e), or 3-oxo-3,4-dihydro-2H-1,2,4-benzothiadiazine1,1dioxides (11d). Subsequently, the 3-oxo compounds (11a-d) can be converted into the corresponding 3-thioxo derivatives (12a-e) by reacting with phosphorus pentasulfide in anhydrous pyridine. Methylation of 12a-e can be accomplished with methyl iodide in a solution of sodium bicarbonate to yield the desired 3-methylsulfide intermediates (13a-e). Nucleophilic substitution of these intermediates with the corresponding primary amine can be accomplished with overnight heating at 130° in a sealed vessel to afford the desired diazoxide derivatives.

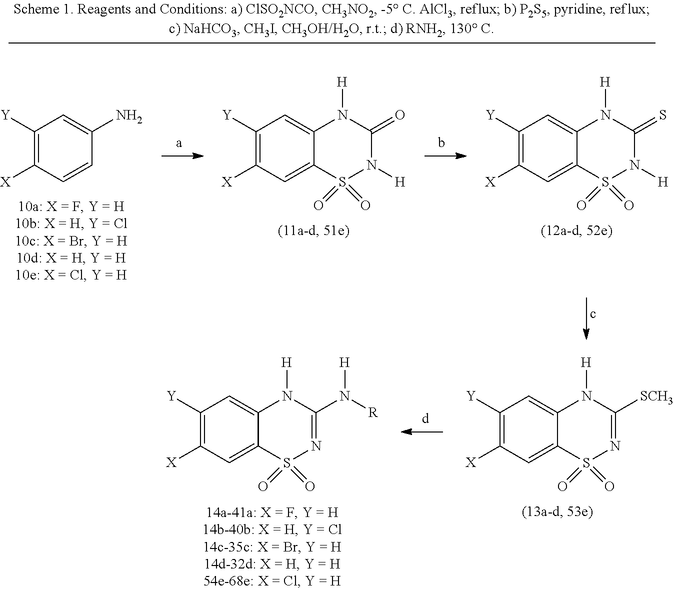

Methylsulfide derivatives (13a-c) can be oxidized to the corresponding methylsulfinyl analog (13aa-ca) (Scheme 2). Subsequently the 3-methyl sulfinyl intermediates can be reacted with cyclopentamine to yield diazoxide derivatives 22a-c.

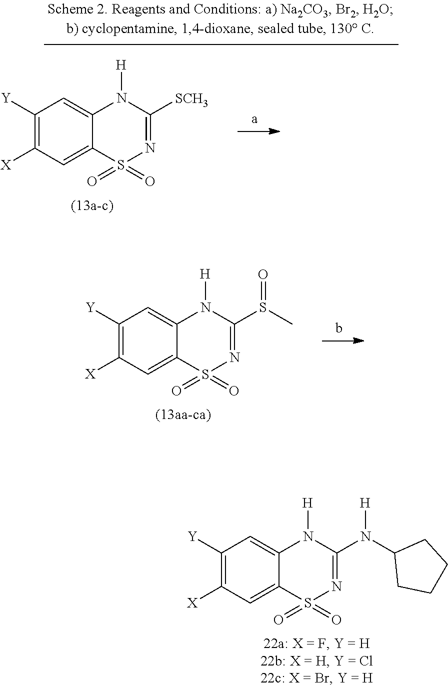

Access to the N-methylated diazoxide derivative (43d) can be achieved (Scheme 3) by exposing methylsulfides 13a and 13d to methyliodide in the presence of base to provide corresponding intermediates 42a and 42d. Subsequent nucleophilic substitution with isopropylamine yields 3-(isopropylamino)-4-methyl-4H-1,2,4benzothiadiazine 1,1-dioxide (43d).

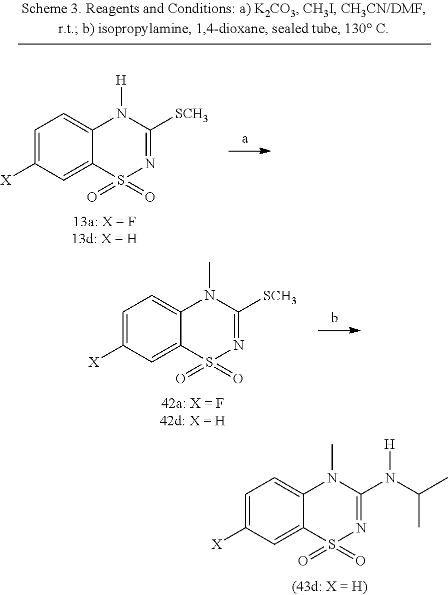

Additional synthetic procedures for preparing the compounds disclosed herein can be found in the Examples section.

### Pharmaceutical Formulations, Dosing, and Routes of Administration

Further provided are pharmaceutical formulations comprising a compound as described herein (e.g., compounds of Formula I, compounds of Table 1, compounds of Table 2, or pharmaceutically acceptable salts of the compounds) and a pharmaceutically acceptable excipient.

The compounds described herein can be administered to a subject in a therapeutically effective amount (e.g., in an amount sufficient to prevent or relieve the symptoms of a disorder associated with aberrant mitochondrial respiratory complex II (CII) or vascular endothelial growth factor (VEGF) activity). The compounds can be administered alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to effect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, e.g., from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

Suitable methods of administering a physiologically-acceptable composition, such as a pharmaceutical composition comprising the compounds disclosed herein (e.g., compounds of Formula I, compounds of Table 1, compounds of Table 2, or pharmaceutically acceptable salts of the compounds), are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by chemico-physical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, Pa., pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the disclosure are useful in fields of human medicine and veterinary medicine. Thus the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

The compounds described herein (e.g., the compounds of Formula I, compounds of Table 1, compounds of Table 2, or pharmaceutically acceptable salts of the compounds) can inhibit a mitochondrial respiratory complex II (CII) or vascular endothelial growth factor (VEGF) pathway. In various embodiments, the compounds are mitochondrial respiratory complex II (CII) or vascular endothelial growth factor (VEGF) modulators, e.g., the compounds change, inhibit, or prevent one or more of the mitochondrial respiratory complex II (CII) or vascular endothelial growth factor (VEGF) pathway's biological activities. In some cases, the VEGF is vascular endothelial growth factor 2 (VEGF2).

The compounds disclosed herein are particularly advantageous for the treatment of diseases or disorders caused by aberrant expression or activity of a mitochondrial respiratory complex II (CII) or vascular endothelial growth factor (VEGF) pathway. The incidence and/or intensity of diseases or disorders associated with aberrant expression or activity of a mitochondrial respiratory complex II (CII) or vascular endothelial growth factor (VEGF) pathway is reduced.

Increased expression and/or activity of a mitochondrial respiratory complex II (CII) or vascular endothelial growth factor (VEGF) pathway includes overexpression or hyperactivity of any component of a mitochondrial respiratory complex II (CII) or vascular endothelial growth factor (VEGF) pathway. Overexpression and/or hyperactivity of the mitochondrial respiratory complex II (CII) or vascular endothelial growth factor (VEGF) pathways is well known to cause many adverse conditions. These include, for example, cancer neurodegenerative diseases and disorders, and eye diseases and disorders. Cancer includes but is not limited to ovarian cancer, breast cancer, prostate cancer, colon cancer, liver cancer, central nervous system (CNS) cancer (e.g., brain cancer), kidney cancer, lung cancer, leukemia, lymphoma, multiple myeloma, thyroid cancer, bone cancer, esophageal cancer, and pancreatic cancer. In some cases, the cancer is a central nervous system (CNS) cancer, prostate cancer, or breast cancer. In some cases, the cancer is prostate cancer or breast cancer. In some cases, the cancer is a central nervous system (CNS) cancer. In some cases, the cancer is prostate cancer. In some cases, the cancer is breast cancer. In some cases, the cancer is triple negative breast cancer. Eye diseases and disorders include but are not limited to retinal vascular disease, coats disease, submacular hemorrhage, wet macular degeneration, and neovascular age-related degeneration. In some cases, the eye disease or disorder is neovascular age-related degeneration.

Mitochondrial respiratory complex II (CII) or vascular endothelial growth factor (VEGF) inhibitors can be used for cancer prevention and treatment.

Compounds of Formula I, compounds of Table 1, compounds of Table 2, or pharmaceutically acceptable salts of the compounds display high selectivity for growth inhibition and/or induction of apoptosis in cancer cells, e.g., in prostate or breast cancer cells.

The disclosed methods include methods for treating disease or disorder capable of being modulated by inhibition of the mitochondrial respiratory complex II (CII) or vascular endothelial growth factor (VEGF) pathway, e.g., cancer, comprising administering to a subject a compound that binds a component of the mitochondrial respiratory complex II (CII) or vascular endothelial growth factor (VEGF) pathway.

Provided herein is a method of modulating the mitochondrial respiratory complex II (CII) or vascular endothelial growth factor (VEGF) pathway in a cell, comprising contacting the cell with a compound or a composition as disclosed herein (e.g., the compounds of Formula I, compounds of Table 1, compounds of Table 2, or pharmaceutically acceptable salts of the compounds) in an amount sufficient to modulate the mitochondrial respiratory complex II (CII) or vascular endothelial growth factor (VEGF) pathway. The contacting of the cell can occur in vitro or in vivo. In some cases, contacting of the cell occurs in vitro. In other cases, contacting of the cell occurs in vivo. Therefore, the disclosure includes administering one or more of a compound described herein to a subject, such as a human, in need thereof. In some embodiments, the subject suffers from a disease or disorder associated with aberrant activity of the mitochondrial respiratory complex II (CII) or vascular endothelial growth factor (VEGF) pathway. Disorders associated with aberrant activity of the mitochondrial respiratory complex II (CII) or vascular endothelial growth factor (VEGF) pathway include, but are not limited to, cancer (e.g., prostate or breast cancer) and eye diseases and disorders. Specifically contemplated cancers include ovarian cancer, breast cancer, prostate cancer, colon cancer, liver cancer, central nervous system (CNS) cancer (e.g., brain cancer), kidney cancer, lung cancer, leukemia, lymphoma, multiple myeloma, thyroid cancer, bone cancer, esophageal cancer, and pancreatic cancer. In some cases, the cancer is a central nervous system (CNS) cancer, prostate cancer, or breast cancer. In some cases, the cancer is prostate cancer or breast cancer. In some cases, the cancer is a central nervous system (CNS) cancer. In some cases, the cancer is prostate cancer. In some cases, the cancer is breast cancer. In some cases, the cancer is triple negative breast cancer. In some cases, the eye disease or disorder is neovascular age-related degeneration.

The disclosed methods utilize compounds that inhibit the mitochondrial respiratory complex II (CII) or vascular endothelial growth factor (VEGF) pathway, for treating, e.g., cancer or eye diseases and disorders. Methods for assessing the usefulness of a compound for treating cancer are known to those of skill in the art. For example, compounds may be assessed using models of cancer, including cells (such as prostate or breast cancer cells), animal models (such as mouse xenograph or other cancer models), or in human subjects having, e.g., prostate or breast cancer.

The compounds described herein can be used to decrease or prevent cancer in subjects with e.g., CNS cancer, prostate cancer, or breast cancer. The compounds described herein can also be used to ameliorate or prevent an eye disease or disorder in subjects with e.g., neovascular age-related degeneration. The subject can be human. In a particular example, a compound or mixture is administered orally, such as by mixing with distilled water. In another example, a compound or mixture is administered intravenously, such as in saline or distilled water. In some examples, treatment with test compound may be a single dose or repeated doses. The test compound may be administered about every 6 hours, about every 12 hours, about every 24 hours (daily), about every 48 hours, about every 72 hours, or about weekly. Treatment with repeated doses may continue for a period of time, for example for about 1 week to 12 months, such as about 1 week to about 6 months, or about 2 weeks to about 3 months, or about 1 to 2 months. Administration of a compound may also continue indefinitely. Doses of test compound are from about 0.1 mg/kg to about 400 mg/kg, such as about 1 mg/kg to about 300 mg/kg, about 2 mg/kg to 200 mg/kg, about 10 mg/kg to about 100 mg/kg, about 20 mg/kg to about 75 mg/kg, or about 25 mg/kg to about 50 mg/kg.

It will be understood that the methods and compositions described herein for treating cancer, comprising administering a compound that inhibits the mitochondrial respiratory complex II (CII) or vascular endothelial growth factor (VEGF) pathway, are applicable to methods of treating other diseases related to mitochondrial respiratory complex II (CII) or vascular endothelial growth factor (VEGF) activity, such as those described above. The methods for assessing the effectiveness of compounds for treating such diseases in cells, appropriate animal models, or affected subjects are known to one of skill in the art.

Uses of the compounds disclosed herein in the preparation of a medicament for treating diseases or disorders related to mitochondrial respiratory complex II (CII) or vascular endothelial growth factor (VEGF) activity also are provided herein.

The disclosure herein will be understood more readily by reference to the following examples, below.

### EXAMPLES

The following examples are provided for illustration and are not intended to limit the scope of the disclosure.

### Example 1: Synthetic Procedures for Compounds of Formula I

General Experimental Procedures.

All reactions were carried out in oven- or flame-dried glassware under positive nitrogen pressure unless otherwise noted. Reaction progress was monitored by thin-layer chromatography (TLC) carried out on silica gel plates (2.5 cm×7.5 cm, 200 μm thick, 60 F254) and visualized by using UV (254 nm) or by potassium permanganate as indictor. Flash column chromatography was performed with silica gel (40-63 μm, 60 Å) or on a Teledyne Isco (CombiFlash R_{f} 200 UV/Vis). Commercial grade solvents and reagents were purchased from Fisher Scientific (Houston, Tex.) or Sigma Aldrich (Milwaukee, Wis.) and were used without further purification except as indicated. Anhydrous solvents were purchased from Across Organics and stored under an atmosphere of dry nitrogen over molecular sieves.

¹H and ¹³C NMR spectra were recorded in the indicated solvent on a Bruker 400 MHz Advance III HD spectrometer at 400 and 100 MHz for ¹H and ¹³C respectively with solvent peak as an internal standard. Multiplicities are indicated by s (single), d (doublet), dd (doublet of doublets), t (triplet), q (quartet), m (multiplet), br (broad). Chemical shifts (6) are reported in parts per million (ppm), and coupling constants (J), in hertz. High-resolution mass spectroscopy was performed on a LC/MS IT-TOF (Shimadzu) using an ESI source conducted at the University of Texas at Arlington, Shimadzu Center for Advanced Analytical Chemistry. High-pressure liquid chromatography was performed on a Gilson HPLC system with 321 pumps and 155 UV/Vis detector using trilution software v2.1 with an ACE Equivalence 3 (C18, 3 μM, 4.6×150 mm) column. All samples were determined to possess >95% purity.

### 7-fluoro-3-oxo-3,4-dihydro-2H-1,2,4-benzothiadiazine 1,1-dioxide (11a)

A solution of chlorosulfonyl isocyanate (2.82 mL, 32.4 mmol) in nitromethane (30 mL) was mixed in a closed dried vessel under nitrogen pressure and cooled at −5° C. (ice and salt bath). To this mixture 4-fluoroaniline (10a, 2.6 mL, 27 mmol) was added slowly. The contents were vigorously stirred for 20 mins followed by the addition of anhydrous AlCl₃ (4.7 g, 35.1 mmol) and the mixture was refluxed for 1 h. The hot solution was poured onto ice (200 g) and stirred for and additional 30 mins and the resulting precipitate was collected by filtration and washed with water. The crude solid was treated with an aqueous solution of sodium bicarbonate (5 g/100 mL) followed by heating until the solid precipitate was dissolved. The solution was treated with charcoal and was filtered, the filtrate solution was adjusted to pH 1 using 12N HCl. The resulting precipitate was filtered, washed with water, and air dried: ¹H NMR (400 MHz, DMSO-d6): δ 7.30 (m, 1H), 7.55 (1H, t, J=8.7 Hz), 7.68 (1H, dd, J=7.5, 2.8 Hz), 11.40 (s, 1H); ¹³C NMR (100 MHz, DMSO-d6): δ 109.27, 119.81, 121.88, 123.58, 132.18, 151.67, 156.58, 159.01; HRMS (ESI) m/z calcd for C₇H₅FN₂O₃S [M+Na]⁺: 238.9902, found: 238.9901.

### 6-chloro-3-oxo-3,4-dihydro-2H-1,2,4-benzothiadiazine1,1-dioxide (11b)

The compound was obtained from 3-chloroaniline (10b, 3.32 mL, 31.35 mmol) by following the experimental conditions described for 11a: ¹H NMR (400 MHz, DMSO-d6): δ 7.26 (1H, d, J=2 Hz), 7.32 (1H, dd, J=8.5, 1.8 Hz), 7.80 (1H, d, J=8.5 Hz), 11.39 (s, 1H); ¹³C NMR (100 MHz, DMSO-d6): δ 116.91, 121.73, 123.96, 124.65, 136.97, 138.54, 151.15; HRMS (ESI) m/z calcd for C₇H₅ClN₂O₃S [M+Na]⁺: 254.9607, found: 254.9606.

### 7-bromo-3-oxo-3,4-dihydro-2H-1,2,4-benzothiadiazine1,1-dioxide (11c)

The compound was obtained from 4-bromoaniline (10c, 3 g, 17.44 mmol) by following the experimental conditions described for 11a with the slight modification that the crude material was dissolved in a 1:1 hydromethanolic solution of sodium bicarbonate instead of an aqueous solution of sodium bicarbonate: ¹H NMR (400 MHz, DMSO-d6): δ 7.19 (1H, d, J=8.7 Hz), 7.78 (1H, dd, J=8.7, 2.2 Hz), 7.91 (1H, d, J=2.2 Hz), 11.46 (s, 1H); ¹³C NMR (100 MHz, DMSO-d6): δ 115.08, 119.83, 124.34, 124.79, 134.86, 137.00, 151.52; HRMS (ESI) m/z calcd for C₇H₅BrN₂O₃S [M+Na]⁺: 298.9101, found: 298.9096.

### 2H-benzo[e][1,2,4]thiadiazin-3(4H)-one 1,1-dioxide (11d)

The compound was obtained from aniline (10d, 4.86 mL, 53.7 mmol) by following the experimental conditions described for 11a: ¹H NMR (400 MHz, DMSO-d6): δ 7.27 (m, 2H), 7.63 (1H, t, J=7.2 Hz), 7.77 (1H, d, J=7.6 Hz), 11.27 (s, 1H); ¹³C NMR (100 MHz, DMSO-d6): δ 117.47, 122.43, 122.97, 123.89, 134.40, 135.48, 151.08; HRMS (ESI) m/z calcd for C₇H₆N₂O₃S [M+Na]⁺: 220.9996, found: 220.9998.

### 7-fluoro-3-thioxo-3,4-dihydro-2H-1,2,4-benzothiadiazine 1,1-dioxide (12a)

A mixture of 7-fluoro-3-oxo-3,4-dihydro-2H-1,2,4-benzothiadiazine1,1-dioxide (11a, 2.8 g, 12.95 mmol) and phosphorus pentasulfide (5.47 g, 12.95 mmol) was dissolved in anhydrous pyridine (50 mL) and refluxed under nitrogen pressure overnight. The reaction was allowed to cool and the solvent removed in vacuo, the crude product was dissolved in an aqueous solution of sodium hydroxide (NaOH) (5 g/100 mL). This solution was treated with charcoal and was filtered. The filtrate was acidified to pH 1 using 12N HCl. The precipitated compound was collected by filtration, washed with water and was allowed to air dry. The dried compound was suspended in an aqueous solution of sodium bicarbonate (NaHCO₃) (10 g/200 mL) and heated until the solid was dissolved. This solution was treated with charcoal and filtered. The filtrate was adjusted to pH 1 using 12N HCl, and the precipitate was collected by filtration, washed with water, and air dried.: ¹H NMR (400 MHz, DMSO-d6): δ 7.29 (m, 1H), 7.56 (m, 1H), 7.68 (1H, dd, J=7.5, 2.8 Hz), 11.35 (s, 1H); ¹³C NMR (100 MHz, DMSO-d6): δ 109.93, 121.01, 122.12, 123.31, 132.56, 158.33, 160.79; HRMS (ESI) m/z calcd for C₇H₅FN₂O₂S₂ [M+Na]⁺: 254.9674, found: 254.9674.

### 6-chloro-3-thioxo-3,4-dihydro-2H-1,2,4-benzothiadiazine 1,1-dioxide (12b)

The compound was obtained from 11b (4.5 g, 19.34 mmol) by following the experimental conditions described for 12a.: ¹H NMR (400 MHz, DMSO-d6): δ 7.26 (1H, d, J=2 Hz), 7.34 (1H, dd, J=8.4, 2 Hz), 7.80 (1H, d, J=8.4 Hz), 11.33 (s, 1H); ¹³C NMR (100 MHz, DMSO-d6): δ 117.76, 121.21, 126.03, 126.95, 137.55, 137.90, 144.72; HRMS (ESI) m/z calcd for C₇H₅ClN₂O₂S₂ [M+Na]⁺: 270.9378, found: 270.9373.

### 7-bromo-3-thioxo-3,4-dihydro-2H-1,2,4-benzothiadiazine 1,1-dioxide (12c)

The compound was obtained from 11c (2.4 g, 8.63 mmol) by following the experimental conditions described for 12a with the slight modification that the crude material was dissolved in 1:1 hydromethanolic solution of sodium bicarbonate instead of an aqueous solution of sodium bicarbonate by heating the mixture until most of the insoluble material dissolved. Charcoal was added to the suspension and filtered. The filtrate was adjusted to pH 1 with 12 N HCl, and the precipitate was collected by filtration, washed with water, and air dried: ¹H NMR (400 MHz, DMSO-d6): δ 5.08 (br, 1H), 7.32 (1H, d, J=8.8 Hz), 7.85 (1H, d, J=8.7 Hz), 7.95 (s, 1H), 11.45 (br, 1H); ¹³C NMR (100 MHz, DMSO-d6): δ 118.22, 120.67, 123.82, 126.08, 135.15, 136.82, 157.57; HRMS (ESI) m/z calcd for C₇H₅BrN₂O₂S₂ [M+Na]⁺: 314.8873, found: 314.8863.

### 2H-benzo[e][1,2,4]thiadiazine-3(4H)-thione 1,1-dioxide (12d)

The compound was obtained from 11d (4.9 g, 24.72 mmol) by following the experimental conditions described for 12a: ¹H NMR (400 MHz, DMSO-d6): δ 7.38 (m, 2H), 7.70 (1H, t, J=7.8 Hz), 7.80 (1H, d, J=7.4 Hz), 12.12 (br, 1H); ¹³C NMR (100 MHz, DMSO-d6): δ 115.52, 121.14, 125.21, 126.83, 136.59, 138.94, 144.16, 144.66; HRMS (ESI) m/z calcd for C₇H₆N₂O₂S₂ [M+Na]⁺: 236.9768, found: 236.9765.

### 7-Fluoro-3-methylsulfanyl-4H-1,2,4-benzothiadiazine 1,1-Dioxide (13a)

7-Fluoro-3-thioxo-3,4dihydro-2H-1,2,4-benzothiadiazine 1,1-dioxide (12a, 2.8 g, 12.06 mmol) was dissolved in a 1:1 hydromethanolic solution of sodium bicarbonate (5 g/200 mL). Methyl iodide was added (1.5 mL, 24.12 mmol) and the solution was stirred for 1 h. The resulting suspension was adjusted to pH 5 using 6N HCl. The suspension was concentrated under reduced pressure, and the precipitate was collected by filtration, washed with water, and air dried: ¹H NMR (400 MHz, DMSO-d6): δ 2.53 (s, 2H), 7.33 (m, 1H), 7.58 (1H, t, J=8.8 Hz), 7.55 (1H, dd, J=7.5, 2.8 Hz), 12.61 (br, 1H); ¹³C NMR (100 MHz, DMSO-d6): δ d=13.85, 109.80, 120.06, 121.91, 122.83, 132.77, 157.84, 161.61; HRMS (ESI) m/z calcd for C₈H₇FN₂O₂S₂ [M+Na]⁺: 268.9831, found: 268.9832.

### 6-Chloro-3-methylsulfanyl-4H-1,2,4-benzothiadiazine 1,1-Dioxide (13b)

The compound was obtained from 12b (2.5, 10.05 mmol) by following the experimental conditions described for 13a: ¹H NMR (400 MHz, DMSO-d6): δ 2.52 (s, 2H), 7.26 (1H, d, J=2 Hz), 7.34 (1H, dd, J=8.5, 2 Hz), 7.80 (1H, d, J=8.5 Hz), 12.61 (s, 1H); ¹³C NMR (100 MHz, DMSO-d6): δ 13.90, 116.83, 120.76, 126.12, 126.48, 137.20, 137.87, 161.88; HRMS (ESI) m/z calcd for C₈H₇ClN₂O₂S₂ [M+Na]⁺: 284.9535, found: 284.9527.

### 7-Bromo-3-methylsulfanyl-4H-1,2,4-benzothiadiazine 1,1-Dioxide (13c)

The compound was obtained from 12c (3.73 g, 12.72 mmol) by following the experimental conditions described for 13a: ¹H NMR (400 MHz, DMSO-d6): δ 2.52 (s, 2H), 7.24 (1H, d, J=8.8 Hz), 7.83 (1H, dd, J=8.7, 2.2 Hz), 7.93 (1H, d, J=2.1 Hz), 12.65 (s, 1H); ¹³C NMR (100 MHz, DMSO-d6): δ 13.88, 117.22, 119.87, 123.43, 125.97, 135.26, 136.60, 161.75; HRMS (ESI) m/z calcd for C₈H₇BrN₂O₂S₂ [M+Na]⁺: 328.90300, found: 328.90244.

### 3-(methylthio)-4H-benzo[e][1,2,4]thiadiazine 1,1-dioxide (13d)

The compound was obtained from 12d (1.92 g, 8.96 mmol) by following the experimental conditions described for 13a: ¹H NMR (400 MHz, DMSO-d6): δ 2.52 (s, 2H), 7.28 (1H, d, J=8.7 Hz), 7.41 (1H, t, J=7.2 Hz), 7.67 (1H, d, J=8.7 Hz), 7.78 (1H, dd, J=7.9, 2 Hz), 12.51 (s, 1H); ¹³C NMR (100 MHz, DMSO-d6): δ 13.98, 117.31, 122.08, 123.85, 126.41, 133.73, 135.99, 161.38; HRMS (ESI) m/z calcd for C₈H₈N₂O₂S₂ [M+Na]⁺: 250.9924, found: 250.9920.

### General Procedures for the Synthesis of 3-(Alkylamino)-7-halo-4H-1,2,4-benzothiadiazine 1,1-Dioxides (14a-a41) (14b-40) (14c-35c) (14d-32d)

Method A.

The appropriate 3-methylsulfanyl-4H-1,2,4-benzothiadiazine1,1-dioxide (13a-13d) (0.25 g) and an appropriate alkylamine (0.7 mL) were dissolved in 1,4-dioxane (8 mL) in a sealed vessel and heated for 24 h at 130° C. The solvent and the excess amine were removed in vacuo, and the residue was dissolved in an aqueous 2% w/v solution of NaOH (7 mL). This solution was treated with charcoal and was filtered. The filtrate was adjusted to pH 1 using 6N HCl. The precipitated compound was collected by filtration, washed with water and air dried. The dried compound was suspended in an aqueous solution of sodium bicarbonate NaHCO₃ (1 g/40 mL). The alkaline solution was treated with charcoal and filtered; the filtrate was adjusted to pH 4-5 with 6N HCl. The precipitate was collected by filtration, washed twice with water, and air dried.

Method B.

A solution of the appropriate 3-methylsulfanyl-4H-1,2,4-benzothiadiazine1,1-dioxide (13a-13d) (0.25 g) and the appropriate amine (5 mL) was heated in a sealed vessel for 48 hr at 120° C. The solvent and excess amine was removed in vacuo, and the residue was dissolved in an aqueous 2% w/v solution of sodium hydroxide (7 mL). This solution was treated with charcoal and was filtered. The filtrate was adjusted to pH 1 using 6N HCl. The precipitated compound was collected by filtration, washed with water and air dried. The dried compound was suspended in an aqueous solution of sodium bicarbonate NaHCO₃ (1 g/40 mL). The alkaline solution was treated with charcoal and filtered, and the filtrate was adjusted to pH 4-5 with 6N HCl. The precipitate was collected by filtration, washed twice with water, and air dried.

### 7-Fluoro-3-isopropylamino-4H-1,2,4-benzothiadiazine 1,1-Dioxide (14a)

The compound was obtained from 13a by following the experimental conditions described for Method A: ¹H NMR (400 MHz, DMSO-d6): δ 1.16 (6H, d, J=6.3 Hz), 3.91 (m, 1H), 7.09 (s, 1H), 7.26 (q, 1H), 7.50 (m, 1H), 10.42 (s, 1H); ¹³C NMR (100 MHz, DMSO-d6): δ 22.66, 43.27, 109.18, 119.50, 120.69, 132.79, 150.96, 156.77, 159.19; HRMS (ESI) m/z calcd for C₁₀H₁₂FN₃O₂S [M+Na]⁺: 280.0532, found: 280.0541.

### 6-Chloro-3-isopropylamino-4H-1,2,4-benzothiadiazine 1,1-Dioxide (14b)

The compound was obtained from 13b by following the experimental conditions described for Method A: ¹H NMR (400 MHz, DMSO-d6): δ 1.18 (6H, d, J=6.4 Hz), 3.93 (m, 1H), 7.09 (br, 1H), 7.27 (m, 1H), 7.47 (m, 1H), 10.40 (s, 1H); ¹³C NMR (100 MHz, DMSO-d6): δ 22.67, 43.26, 109.18, 119.50, 120.68, 132.79, 150.96, 156.77, 159.19; HRMS (ESI) m/z calcd for C₁₀H₁₂ClN₃O₂S [M+Na]⁺: 296.0236, found: 296.0237.

### 7-Bromo-3-isopropylamino-4H-1,2,4-benzothiadiazine 1,1-Dioxide (14c)

The compound was obtained from 13c by following the experimental conditions described for Method A: ¹H NMR (400 MHz, DMSO-d6): δ 1.16 (6H, d, J=6.5 Hz), 3.91 (m, 1H), 7.16 (2H, d, J=8.3 Hz), 7.70 (1H, dd, J=8.7, 2.1 Hz), 7.76 (1H, d, J=2.1 Hz), 10.48 (s, 1H); ¹³C NMR (100 MHz, DMSO-d6): δ 22.62, 43.30, 114.97, 119.59, 124.61, 125.34, 135.42, 145.54, 150.68; HRMS (ESI) m/z calcd for C₁₀H₁₂BrN₃O₂S [M+Na]⁺: 339.9731, found: 339.9716.

### 3-(isopropylamino)-4H-benzo[e][1,2,4]thiadiazine 1,1-dioxide (14d)

The compound was obtained from 13d by following the experimental conditions described for Method A: ¹H NMR (400 MHz, DMSO-d6 δ 1.16 (6H, d, J=6.5 Hz), 3.93 (m, 1H), 6.97 (s, 1H), 7.18 (1H, d, J=8.2 Hz), 7.24 (1H, t, J=7.8 Hz), 7.54 (1H, t, J=8.7 Hz), 7.65 (1H, dd, J=7.8, 2 Hz), 10.31 (s, 1H); ¹³C NMR (100 MHz, DMSO-d6): δ 22.70, 43.13, 116.96, 123.12, 123.24, 124.10, 132.78, 136.10, 150.82; HRMS (ESI) m/z calcd for C₁₀H₁₃N₃O₂S [M+Na]⁺: 262.0626, found: 262.0629.

### 7-fluoro-3-((3-methylbutan-2-yl)amino)-4H-benzo[e][1,2,4]thiadiazine 1,1-dioxide (15a)

The compound was obtained from 13a by following the experimental conditions described for Method B: ¹H NMR (400 MHz, DMSO-d6): δ 0.87 (q, 6H), 1.09 (3H, d, J=6.6 Hz), 1.74 (m, 1H), 3.69 (m, 1H), 6.96 (br, 1H), 7.24 (br, 1H), 7.49 (m, 2H), 10.32 (s, 1H); ¹³C NMR (100 MHz, DMSO-d6): δ 17.47, 18.70, 18.97, 32.70, 52.08, 109.19, 119.39, 120.67, 132.70, 124.10, 151.42, 156.77, 159.19; HRMS (ESI) m/z calcd for C₁₂H₁₆FN₃O₂S [M+Na]⁺: 308.0844 found: 308.0844.

### 6-chloro-3-((3-methylbutan-2-yl)amino)-4H-benzo[e][1,2,4]thiadiazine 1,1-dioxide (15b)

The compound was obtained from 13b by following the experimental conditions described for Method B: ¹H NMR (400 MHz, DMSO-d6): δ 0.90 (q, 6H), 1.08 (3H, d, J=6.6 Hz), 1.74 (m, 1H), 3.69 (m, 1H), 6.96 (br, 1H), 7.24 (br, 1H), 7.50 (m, 2H), 10.31 (s, 1H); ¹³C NMR (100 MHz, DMSO-d6): δ 17.48, 18.70, 18.97, 32.70, 52.08, 109.19, 119.39, 120.43, 132.71, 151.42, 156.77, 159.19; HRMS (ESI) m/z calcd for C₁₂H₁₆ClN₃O₂S [M+Na]⁺: 324.0550 found: 324.0565.

### 7-bromo-3-((3-methylbutan-2-yl)amino)-4H-benzo[e][1,2,4]thiadiazine 1,1-dioxide (15c)

The compound was obtained from 13c by following the experimental conditions described for Method B: ¹H NMR (400 MHz, DMSO-d6): δ 0.89 (q, 6H), 1.08 (3H, d, J=6.6 Hz), 1.76 (m, 1H), 3.71 (m, 1H), 6.99 (br, 1H), 7.16 (1H, d, J=8.7 Hz), 7.72 (1H, dd, J=8.7, 1.9 Hz), 7.75 (1H, d, J=1.9 Hz), 10.37 (s, 1H); ¹³C NMR (100 MHz, DMSO-d6): δ 17.45, 18.71, 18.97, 32.68, 52.11, 114.95, 119.57, 124.68, 125.35, 135.36, 135.52, 151.16; HRMS (ESI) m/z calcd for C₁₂H₁₆BrN₃O₂S [M+Na]⁺: 368.00443 found: 368.004.

### 3-((3-methylbutan-2-yl)amino)-4H-benzo[e][1,2,4]thiadiazine 1,1-dioxide (15d)

The compound was obtained from 13d by following the experimental conditions described for Method B: ¹H NMR (400 MHz, DMSO-d6): δ 0.90 (q, 6H), 1.10 (3H, d, J=6.6 Hz), 1.75 (m, 1H), 3.72 (m, 1H), 6.88 (br, 1H), 7.16 (1H, d, J=7.4 Hz), 7.24 (1H, t, J=8.1 Hz), 7.54 (1H, t, J=8.2 Hz), 7.66 (1H, dd, J=7.8, 2 Hz), 10.23 (s, 1H); ¹³C NMR (100 MHz, DMSO-d6): δ 17.47, 18.69, 18.97, 32.68, 51.89, 116.93, 123.17, 123.25, 124.09, 132.78, 136.04, 151.27; HRMS (ESI) m/z calcd for C₁₂H₁₇N₃O₂S [M+Na]⁺: 290.0939 found: 290.0947.

### 3-((4-chlorobenzyl)amino)-7-fluoro-4H-benzo[e][1,2,4]thiadiazine 1,1-dioxide (16a)

The compound was obtained from 13a by following the experimental conditions described for Method A: ¹H NMR (400 MHz, DMSO-d6): δ 4.46 (s, 2H), 7.28 (q, 1H), 7.36 (2H, d, J=8.5 Hz), 7.41-7.52 (m, 4H), 7.70 (br, 1H), 10.89 (s, 1H); ¹³C NMR (100 MHz, DMSO-d6): δ 43.67, 109.48, 119.62, 120.80, 123.77, 127.64, 128.78, 129.50, 132.07, 132.94, 138.15, 151.82, 156.83, 159.25; HRMS (ESI) m/z calcd for C₁₄H₁₁ClFN₃O₂S [M+Na]⁺: 362.01422 found: 362.0137.

### 6-chloro-3-((4-chlorobenzyl)amino)-4H-benzo[e][1,2,4]thiadiazine 1,1-dioxide (16b)

The compound was obtained from 13b by following the experimental conditions described for Method A: ¹H NMR (400 MHz, DMSO-d6): δ 4.47 (2H, d, J=5.8 Hz), 7.29 (q, 2H), 7.37 (2H, d, J=8.5 Hz), 7.41 (2H, d, J=8.5 Hz), 7.68 (1H, d, J=8.5 Hz), 7.70 (br, 1H) 10.84 (br, 1H); ¹³C NMR (100 MHz, DMSO-d6): δ 43.67, 116.64, 121.84, 124.34, 125.45, 128.80, 129.54, 131.31, 132.11, 137.01, 137.53, 138.00, 151.47; HRMS (ESI) m/z calcd for C₁₄H₁₁Cl₂N₃O₂S [M+Na]⁺: 377.9846 found: 377.9839.

### 7-bromo-3-((4-chlorobenzyl)amino)-4H-benzo[e][1,2,4]thiadiazine 1,1-dioxide dioxide (16c)

The compound was obtained from 13c by following the experimental conditions described for Method A: ¹H NMR (400 MHz, DMSO-d6): δ 4.46 (2H, d, J=5.8 Hz), 7.19 (1H, d, J=8.7 Hz), 7.36 (2H, d, J=8.7 Hz), 7.40 (2H, d, J=8.7 Hz), 7.78 (m, 3H), 7.70 (s, 1H), 11.01 (s, 1H); ¹³C NMR (100 MHz, DMSO-d6): δ 43.68, 115.14, 119.63, 124.53, 125.41, 128.80, 129.52, 132.11, 135.45, 135.66, 138.01, 151.52; HRMS (ESI) m/z calcd for C₁₄H₁₁ClBrN₃O₂S [M+Na]⁺: 421.9341 found: 421.93244.

### 3-((4-chlorobenzyl)amino)-4H-benzo[e][1,2,4]thiadiazine 1,1-dioxide (16d)

The compound was obtained from 13d by following the experimental conditions described for Method A: ¹H NMR (400 MHz, DMSO-d6): δ 4.47 (2H, d, J=5.8 Hz), 7.21 (1H, d, J=8.5 Hz), 7.26 (1H, t, J=7.8 Hz), 7.37 (2H, d, J=8.5 Hz), 7.41 (2H, d, J=8.5 Hz), 7.56 (1H, t, J=8.3 Hz), 7.61 (br, 1H), 7.65 (1H, dd, J=7.8, 2 Hz), 10.85 (s, 1H); ¹³C NMR (100 MHz, DMSO-d6): δ 43.57, 117.02, 119.63, 123.05, 123.30, 124.26, 128.79, 129.51, 132.05, 132.89, 136.13, 138.22, 151.62; HRMS (ESI) m/z calcd for C₁₄H₁₁ClN₃O₂S [M+Na]⁺: 344.0236 found: 344.0236.

### 7-fluoro-3-((4-fluorobenzyl)amino)-4H-benzo[e][1,2,4]thiadiazine 1,1-dioxide (17a)

The compound was obtained from 13a by following the experimental conditions described for Method A: ¹H NMR (400 MHz, DMSO-d6): δ 4.46 (s, 2H), 7.16 (2H, t, J=8.8 Hz), 7.27 (q, 1H), 7.38 (m, 2H), 7.45 (1H, t, J=8.8 Hz), 7.52 (1H, dd, J=7.5, 2.8 Hz), 7.68 (br, 1H), 10.68 (br, 1H); ¹³C NMR (100 MHz, DMSO-d6): δ 43.66, 109.23, 115.48, 119.60, 120.77, 123.80, 129.77, 132.92, 135.20, 151.80, 156.84, 159.26, 160.58, 163.00; HRMS (ESI) m/z calcd for C₁₄H₁₁F2N₃O₂S [M+Na]⁺: 346.0437 found: 346.0428.

### 6-chloro-3-((4-fluorobenzyl)amino)-4H-benzo[e][1,2,4]thiadiazine 1,1-dioxide (17b)

The compound was obtained from 13b by following the experimental conditions described for Method A: ¹H NMR (400 MHz, DMSO-d6): δ 4.47 (s, 2H), 7.18 (2H, t, J=8.8 Hz), 7.28 (q, 1H), 7.39 (q, 2H), 7.46 (1H, t, J=8.8 Hz), 7.53 (1H, dd, J=7.5, 2.8 Hz), 7.67 (br, 1H), 10.83 (br, 1H); ¹³C NMR (100 MHz, DMSO-d6): δ 43.65, 109.50, 115.69, 119.50, 120.79, 123.86, 129.76, 132.84, 135.19, 151.75, 156.85, 159.27, 160.58, 163.00; HRMS (ESI) m/z calcd for C₁₄H₁₁ClFN₃O₂S [M+Na]⁺: 362.0142 found: 362.0160.

### 7-bromo-3-((4-fluorobenzyl)amino)-4H-benzo[e][1,2,4]thiadiazine 1,1-dioxide (17c)

The compound was obtained from 13c by following the experimental conditions described for Method A: ¹H NMR (400 MHz, DMSO-d6): δ 4.45 (2H, d, J=5.8 Hz), 7.18 (3H, d, J=8.8 Hz), 7.38 (q, 2H), 7.72 (2H, dd, J=8.7, 2.2 Hz), 7.78 (1H, d, J=2.2 Hz), 10.94 (s, 1H); ¹³C NMR (100 MHz, DMSO-d6): δ 43.66, 115.12, 115.50, 115.71, 119.63, 124.56, 125.40, 129.77, 135.11, 135.46, 135.65, 151.49, 160.59, 163.00; HRMS (ESI) m/z calcd for C₁₄H₁₁BrFN₃O₂S [M+Na]⁺: 405.963708 found: 405.96021.

### 3-((4-fluorobenzyl)amino)-4H-benzo[e][1,2,4]thiadiazine 1,1-dioxide (17d)

The compound was obtained from 13d by following the experimental conditions described for Method A: ¹H NMR (400 MHz, DMSO-d6): δ 4.45 (2H, d, J=5.8 Hz), 7.18 (3H, t, J=8.8 Hz), 7.26 (2H, d, J=7.4 Hz), 7.39 (q, 2H), 7.55 (2H, t, J=7.2 Hz), 7.69 (1H, d, J=7.8 Hz), 10.77 (s, 1H); ¹³C NMR (100 MHz, DMSO-d6): δ 43.56, 115.49, 115.70, 117.02, 123.07, 123.70, 124.24, 129.77, 132.87, 135.30, 136.13, 151.59, 160.58, 162.99; HRMS (ESI) m/z calcd for C₁₄H₁₂FN₃O₂S [M+Na]⁺: 328.0531 found: 328.0530.

### 7-fluoro-3-(phenethylamino)-4H-benzo[e][1,2,4]thiadiazine 1,1-dioxide (18a)

The compound was obtained from 13a by following the experimental conditions described for Method A: ¹H NMR (400 MHz, DMSO-d6): δ 2.86 (2H, t, J=7.2 Hz), 3.48 (q, 2H), 7.20-7.34 (m, 7H), 7.45 (1H, t, J=7.3 Hz), 7.52 (1H, dd, J=7.5, 2.8 Hz), 10.55 (s, 1H); ¹³C NMR (100 MHz, DMSO-d6): δ 35.08, 43.42, 109.23, 119.47, 120.49, 120.72, 123.79, 126.74, 128.90, 129.15, 132.85, 139.32, 151.69, 156.76, 159.21; HRMS (ESI) m/z calcd for C₁₅H₁₄FN₃O₂S [M+Na]⁺: 342.06884 found: 342.0687.

### 6-chloro-3-(phenethylamino)-4H-benzo[e][1,2,4]thiadiazine 1,1-dioxide (18b)

The compound was obtained from 13b by following the experimental conditions described for Method A: ¹H NMR (400 MHz, DMSO-d6): δ 2.86 (2H, t, J=7.2 Hz), 3.47 (q, 2H), 7.20-7.34 (m, 8H), 7.68 (1H, dd, J=8.4 Hz), 10.71 (br, 1H); ¹³C NMR (100 MHz, DMSO-d6): δ 35, 42.43, 116.52, 121.86, 124.22, 125.43, 126.74, 128.89, 129.15, 132.85, 136.94, 137.52, 139.29, 151.39; HRMS (ESI) m/z calcd for C₁₅H₁₄ClN₃O₂S [M+Na]⁺: 358.0392 found: 358.0388.

### 7-bromo-3-(phenethylamino)-4H-benzo[e][1,2,4]thiadiazine 1,1-dioxide (18c)

The compound was obtained from 13c by following the experimental conditions described for Method A: ¹H NMR (400 MHz, DMSO-d6): δ 2.86 (2H, t, J=7.3 Hz), 3.48 (q, 2H), 7.15-7.34 (m, 7H), 7.70 (1H, dd, J=8.7, 2.2 Hz), 7.77 (1H, d, J=2.2 Hz), 10.79 (br, 1H); ¹³C NMR (100 MHz, DMSO-d6): δ 35.03, 42.42, 115.01, 119.51, 124.52, 125.39, 126.75, 128.90, 129.14, 135.44, 135.58, 139.27, 151.43; HRMS (ESI) m/z calcd for C₁₅H₁₄BrN₃O₂S [M+Na]⁺: 401.98878 found: 401.98571.

### 3-(ethylamino)-7-fluoro-4H-benzo[e][1,2,4]thiadiazine 1,1-dioxide (19a)

The compound was obtained from 13a by following the experimental conditions described for Method A: ¹H NMR (400 MHz, DMSO-d6): δ 1.12 (3H, t, J=7.1 Hz), 3.26 (m, 2H), 7.18 (br, 1H), 7.26 (m, 1H), 7.42 (m, 1H), 7.49 (1H, dd, J=7.5, 2.8 Hz), 10.66 (s, 1H); ¹³C NMR (100 MHz, DMSO-d6): δ 14.99, 36.03, 109.42, 119.44, 123.82, 132.89, 151.59, 156.78, 159.71; HRMS (ESI) m/z calcd for C₈H₁₀FN₃O₂S [M+Na]⁺: 266.037547 found: 266.0385.

### 6-chloro-3-(ethylamino)-4H-benzo[e][1,2,4]thiadiazine 1,1-dioxide (19b)

The compound was obtained from 13b by following the experimental conditions described for Method A: ¹H NMR (400 MHz, DMSO-d6): δ 1.12 (3H, t, J=7.1 Hz), 3.25 (m, 2H), 7.19 (br, 1H), 7.27 (m, 1H), 7.42-7.50 (m, 2H), 10.66 (s, 1H); ¹³C NMR (100 MHz, DMSO-d6): δ 15.00, 36.03, 109.43, 119.44, 120.45, 132.88, 151.58, 156.75, 159.17; HRMS (ESI) m/z calcd for C₈H₁₀ClN₃O₂S [M+Na]⁺: 282.0079 found: 282.0110.

### 7-bromo-3-(ethylamino)-4H-benzo[e][1,2,4]thiadiazine 1,1-dioxide (19c)

The compound was obtained from 13c by following the experimental conditions described for Method A: ¹H NMR (400 MHz, DMSO-d6): δ 1.12 (3H, t, J=7.1 Hz), 3.25 (m, 2H), 7.19 (1H, d, J=8.5 Hz), 7.23 (br, 1H), 7.71 (1H, dd, J=8.7, 2.2 Hz), 7.75 (1H, d, J=2.2 Hz), 10.72 (s, 1H); ¹³C NMR (100 MHz, DMSO-d6): δ 14.96, 36.05, 114.92, 119.54, 124.60, 125.35, 135.54, 151.33; HRMS (ESI) m/z calcd for C₈H₁₀BrN₃O₂S [M+Na]⁺: 325.95748 found: 325.95503.

### 3-(ethylamino)-4H-benzo[e][1,2,4]thiadiazine 1,1-dioxide (19d)

The compound was obtained from 13d by following the experimental conditions described for Method A: ¹H NMR (400 MHz, DMSO-d6): δ 1.12 (3H, t, J=7.1 Hz), 3.25 (m, 2H), 7.07 (br, 1H), 7.19 (1H, d, J=8 Hz), 7.24 (1H, t, J=8 Hz), 7.53 (1H, t, J=8.7 Hz), 7.64 (1H, dd, J=7.8, 2 Hz), 10.56 (s, 1H); ¹³C NMR (100 MHz, DMSO-d6): δ 15.03, 35.93, 116.90, 123.09, 123.25, 124.06, 132.76, 136.21, 151.45; HRMS (ESI) m/z calcd for C₈H₁₀BrN₃O₂S [M+Na]⁺: 248.046969 found: 248.0466.

### 7-fluoro-3-((4-methoxybenzyl)amino)-4H-benzo[e][1,2,4]thiadiazine 1,1-dioxide (20a)

The compound was obtained from 13a by following the experimental conditions described for Method A: ¹H NMR (400 MHz, DMSO-d6): ¹H NMR (400 MHz, DMSO-d6): δ 3.73 (s, 3H), 4.40 (s, 2H), 6.92 (2H, d, J=8.5 Hz), 7.27 (m, 3H), 7.45 (2H, t, J=8.7 Hz), 7.51 (1H, dd, J=7.5, 2.8 Hz) 7.60 (br, 1H); ¹³C NMR (100 MHz, DMSO-d6): δ 43.85, 55.51, 109.21, 114.25, 119.54, 120.48, 120.72, 123.81, 129.14, 130.83, 133.06, 151.80, 156.77, 158.90, 159.19; HRMS (ESI) m/z calcd for C₁₅H₁₄FN₃O₃S [M+Na]⁺: 358.063762 found: 358.0616.

### 6-chloro-3-((4-methoxybenzyl)amino)-4H-benzo[e][1,2,4]thiadiazine 1,1-dioxide (20b)

The compound was obtained from 13b by following the experimental conditions described for Method A: ¹H NMR (400 MHz, DMSO-d6): δ 3.73 (s, 3H), 4.40 (s, 2H), 6.92 (2H, d, J=8.5 Hz), 7.28 (m, 4H), 7.69 (1H, d, J=8.7 Hz), 7.74 (br, 1H); ¹³C NMR (100 MHz, DMSO-d6): δ 43.87, 55.51, 114.25, 116.63, 121.87, 124.23, 125.41, 129.19, 130.65, 130.90, 137.01, 137.60, 151.40, 158.93; HRMS (ESI) m/z calcd for C₁₅H₁₄ClN₃O₃S [M+Na]⁺: 374.034212 found: 374.0332.

### 7-bromo-3-((4-methoxybenzyl)amino)-4H-benzo[e][1,2,4]thiadiazine 1,1-dioxide (20c)

The compound was obtained from 13c by following the experimental conditions described for Method A: ¹H NMR (400 MHz, DMSO-d6): δ 3.73 (s, 3H), 4.39 (2H, d, J=5.8 Hz), 6.92 (m, 2H), 7.19 (1H, d, J=8.7 Hz), 7.26 (2H, d, J=8.7 Hz), 7.65 (br, 1H), 7.71 (1H, dd, J=8.7, 2.2 Hz), 7.78 (1H, d, J=2.2 Hz), 10.85 (s, 1H); ¹³C NMR (100 MHz, DMSO-d6): δ 43.86, 55.54, 114.28, 115.05, 119.59, 124.59, 125.39, 129.17, 130.66, 135.47, 135.61, 151.53, 158.94; HRMS (ESI) m/z calcd for C₁₅H₁₄BrN₃O₃S [M+Na]⁺: 417.983695 found: 417.98112.

### 3-((4-methoxybenzyl)amino)-4H-benzo[e][1,2,4]thiadiazine 1,1-dioxide (20d)

The compound was obtained from 13d by following the experimental conditions described for Method A: ¹H NMR (400 MHz, DMSO-d6): δ 3.73 (s, 3H), 4.39 (2H, d, J=5.8 Hz), 6.93 (1H, d, J=8.7 Hz), 7.18 (1H, d, J=8.2 Hz), 7.37 (m, 3H), 7.49 (br, 1H), 7.55 (2H, t, J=8.3 Hz), 7.66 (1H, dd, J=7.8, 2 Hz), 10.68 (s, 1H); ¹³C NMR (100 MHz, DMSO-d6): δ 43.75, 55.53, 114.27, 116.98, 123.10, 123.30, 124.18, 129.16, 130.85, 132.84, 136.15, 151.53, 158.91; HRMS (ESI) m/z calcd for C₁₅H₁₅N₃O₃S [M+Na]⁺: 340.073184 found: 340.0722.

### 3-(benzylamino)-7-fluoro-4H-benzo[e][1,2,4]thiadiazine 1,1-dioxide (21a)

The compound was obtained from 13a by following the experimental conditions described for Method A: ¹H NMR (400 MHz, DMSO-d6): δ 4.48 (2H, d, J=5.8 Hz), 7.26 (m, 2H), 7.34 (m, 4H), 7.51 (m, 2H) 7.65 (br, 1H), 10.70 (br, 1H); ¹³C NMR (100 MHz, DMSO-d6): δ 44.35, 109.25, 109.49, 119.45, 120.54, 123.87, 127.84, 129.07, 132.94, 138.97, 151.86, 156.83, 159.25; HRMS (ESI) m/z calcd for C₁₄H₁₂FN₃O₂S [M+Na]⁺: 328.0531 found: 328.0523.

### 3-(benzylamino)-6-chloro-4H-benzo[e][1,2,4]thiadiazine 1,1-dioxide (21b)

The compound was obtained from 13b by following the experimental conditions described for Method A: ¹H NMR (400 MHz, DMSO-d6): δ 4.48 (s, 2H), 7.29 (m, 3H), 7.34 (m, 4H) 7.69 (1H, d, J=8.8 Hz), 7.82 (br, 1H), 10.46 (br, 1H); ¹³C NMR (100 MHz, DMSO-d6): δ 44.34, 116.69, 121.88, 124.23, 125.42, 127.58, 127.68, 128.87, 136.99, 137.69, 138.86, 151.57; HRMS (ESI) m/z calcd for C₁₄H₁₂ClN₃O₂S [M+Na]⁺: 344.0236 found: 344.0235.

### 3-(benzylamino)-7-bromo-4H-benzo[e][1,2,4]thiadiazine 1,1-dioxide (21c)

The compound was obtained from 13c by following the experimental conditions described for Method A: ¹H NMR (400 MHz, DMSO-d6): δ 4.48 (2H, d, J=5.8 Hz), 7.21 (1H, d, J=8.7 Hz), 7.27 (m, 1H), 7.34 (m, 4H), 7.72 (2H, dd, J=8.7, 2.2 Hz), 7.78 (1H, d, J=2.2 Hz), 10.87 (s, 1H); ¹³C NMR (100 MHz, DMSO-d6): δ 44.33, 115.08, 119.63, 124.57, 125.39, 127.59, 127.67, 128.87, 135.50, 135.64, 138.84, 151.57; HRMS (ESI) m/z calcd for C₁₄H₁₂BrN₃O₂S [M+Na]⁺: 387.97313 found: 387.97103.

### 3-(benzylamino)-4H-benzo[e][1,2,4]thiadiazine 1,1-dioxide (21d)

The compound was obtained from 13d by following the experimental conditions described for Method A: ¹H NMR (400 MHz, DMSO-d6 δ 4.49 (2H, d, J=5.8 Hz), 7.22 (1H, d, J=8.2 Hz), 7.26 (m, 2H), 7.34 (4H, d, J=4.5 Hz), 7.55 (2H, t, J=8.3 Hz), 7.70 (1H, d, J=7.8 Hz), 10.74 (s, 1H); ¹³C NMR (100 MHz, DMSO-d6): δ 44.26, 117.03, 123.10, 123.31, 124.22, 127.56, 127.67, 128.87, 136.16, 139.03, 151.65; HRMS (ESI) m/z calcd for C₁₄H₁₃N₃O₂S [M+Na]⁺: 310.0626 found: 310.0621.

### 7-fluoro-3-(methylsulfinyl)-4H-benzo[e][1,2,4]thiadiazine 1,1-dioxide (13aa)

The 7-fluoro-3-methylsulfanyl-4H-1,2,4-benzothiadiazine1,1-dioxide (13a, 0.5 g, 2.03 mmol) was suspended in an aqueous solution of sodium carbonate (2.2 g/25 ml) and the aqueous solution 2 N NaOH was added until the mixture was completely dissolved. At room temperature, bromine (0.2 mL, 2.03 mmol) was added under vigorous stirring for 30 min, the resulting suspension was adjusted to pH 2-3 by adding 6 N HCl. The insoluble compound was collected by filtration, washed twice with water, and suspended under stirring in methanol (10 mL). The resultant precipitate was collected by filtration, washed with water and methanol, and dried: ¹H NMR (400 MHz, DMSO-d6): δ 3.45 (s, 3H), 7.66 (m, 1H), 7.76-7-81 (m, 2H); HRMS (ESI) m/z calcd for C₈H₇FN₂O₃S₂ [M+Na]⁺: 284.9780, found: 284.9831.

### 6-chloro-3-(methylsulfinyl)-4H-benzo[e][1,2,4]thiadiazine 1,1-dioxide (13ba)

The compound was obtained from 13b (1 g, 3.81 mmol) by following the experimental conditions described for 13aa: ¹H NMR (400 MHz, DMSO-d6): δ 3.44 (s, 3H), 7.66 (m, 1H), 7.74-7-80 (m, 2H) (s, 1H); HRMS (ESI) m/z calcd for C₈H₇ClN₂O₃S₂ [M+Na]⁺: 300.9484, found: 300.9526.

### 7-bromo-3-(methylsulfinyl)-4H-benzo[e][1,2,4]thiadiazine 1,1-dioxide (13ca)

The compound was obtained from 13c (1 g, 3.26 mmol) by following the experimental conditions described for 13aa: ¹H NMR (400 MHz, DMSO-d): δ 3.45 (s, 3H), 7.66 (m, 1H), 7.76-7-81 (m, 1H); HRMS (ESI) m/z calcd for C₈H₇BrN₂O₂S₂ [M+Na]⁺: 328.9030, found: 328.9018.

### 7-fluoro-3-(cyclopentylamino)-4H-benzo[e][1,2,4]thiadiazine 1,1-dioxide (22a)

A mixture of 7-fluoro-3-methylsulfinyl4H-1,2,4-benzothiadiazine 1,1-dioxide (13aa) (0.25 g, 0.953 mmol) and cyclopentylamine (0.3 ml, 2.89 mmol) was dissolved in 1,4-Dioxane (5 mL) and heated in a sealed vessel overnight at 160° C. The solvent and excess amine was removed in vacuo, and the residue was dissolved in a hydromethanolic (1:1) 2% w/v solution of NaOH (10 mL). The alkaline solution was treated with charcoal and was filtered, and the filtrate was adjusted to pH 4-5 with 6N HCl. The precipitate was collected by filtration, washed with water, and air dried. The dried compound was suspended in an aqueous solution of sodium bicarbonate NaHCO₃ (1 g/40 mL). The alkaline solution was treated with charcoal and filtered, and the filtrate was adjusted to pH 4-5 with 6 N HCl. The precipitate was collected by filtration, washed with water, and air dried. The compound was recrystallized from methanol/water: ¹H NMR (400 MHz, DMSO-d6): δ 1.46-1.66 (m, 6H), 1.90 (m, 2H), 4.07 (m, 1H), 7.27 (s, 2H), 7.45 (m, 2H), 10.35 (s, 1H); ¹³C NMR (100 MHz, DMSO-d6): δ 23.72, 32.71, 52.82, 109.42, 119.46, 120.66, 132.74, 151.31, 156.78, 159.20; HRMS (ESI) m/z calcd for C₁₂H₁₄FN₃O₂S [M+Na]⁺: 306.0688, found: 306.0675.

### 6-chloro-3-(cyclopentylamino)-4H-benzo[e][1,2,4]thiadiazine 1,1-dioxide (22b)

The compound was obtained from 13ba (0.25 g, 0.897 mmol) by following the experimental conditions described for 22a: ¹H NMR (400 MHz, DMSO-d6): δ 1.48-1.67 (m, 6H), 1.91 (m, 2H), 4.06 (m, 1H), 7.29 (2H, dd, J=8.5, 2 Hz), 7.39 (br, 1H), 7.69 (1H, d, J=8.7 Hz), 10.32 (s, 1H); ¹³C NMR (100 MHz, DMSO-d6): δ 22.65, 32.66, 52.85, 116.63, 121.98, 124.23, 125.38, 136.91, 137.44, 150.99; HRMS (ESI) m/z calcd for C₁₂H₁₄ClN₃O₂S [M+Na]⁺: 322.0392, found: 322.0407.

### 7-bromo-3-(cyclopentylamino)-4H-benzo[e][1,2,4]thiadiazine 1,1-dioxide (22c)

The compound was obtained from 13ca (0.25 g, 0.77 mmol) by following the experimental conditions described for 22a: ¹H NMR (400 MHz, DMSO-d6): δ 1.48-1.67 (m, 6H), 1.91 (m, 2H), 4.06 (m, 1H), 7.19 (br, 2H), 7.28 (br, 1H), 7.76 (m, 1H), 10.40 (s, 1H); ¹³C NMR (100 MHz, DMSO-d6): δ 23.64, 32.69, 52.84, 114, 99, 119.63, 124.65, 125.34, 135.40, 135.54, 151.05; HRMS (ESI) m/z calcd for C₁₂H₁₄BrN₃O₂S [M+Na]⁺: 365.98878, found: 365.98796.

### 7-fluoro-3-((3-phenylpropyl)amino)-4H-benzo[e][1,2,4]thiadiazine 1,1-dioxide (23a)

The compound was obtained from 13a by following the experimental conditions described for Method A: ¹H NMR (400 MHz, DMSO-d6): δ 1.83 (m, 2H), 2.63 (2H, t, J=7.8 Hz), 3.24 (q, 2H) 7.16-7.30 (m, 7H), 7.42-7.51 (m, 2H) 10.68 (br, 1H); ¹³C NMR (100 MHz, DMSO-d6): δ 31.01, 32.78, 109.20, 109.45, 119.40, 120.47, 120.70, 123.90, 126.28, 128.79, 132.87, 141.86, 151.75, 156.78, 159.19; HRMS (ESI) m/z calcd for C₁₆H₁₆FN₃O₂S [M+Na]⁺: 356.084497 found: 356.0811.

### 6-chloro-3-((3-phenylpropyl)amino)-4H-benzo[e][1,2,4]thiadiazine 1,1-dioxide (23b)

The compound was obtained from 13b by following the experimental conditions described for Method A: ¹H NMR (400 MHz, DMSO-d6): δ 1.83 (m, 2H), 2.63 (2H, t, J=7.8 Hz), 3.23 (q, 2H) 7.17-7.37 (m, 8H), 7.67 (1H, d, J=8.5 Hz), 10.66 (s, 1H); ¹³C NMR (100 MHz, DMSO-d6): δ 30.93, 31.16, 32.79, 116.59, 121.92, 124.18, 125.40, 126.29, 128.75, 128.80, 136.90, 137.60, 141.86, 151.44; HRMS (ESI) m/z calcd for C₁₆H₁₆ClN₃O₂S [M+Na]⁺: 372.054947 found: 372.0544.

### 7-bromo-3-((3-phenylpropyl)amino)-4H-benzo[e][1,2,4]thiadiazine 1,1-dioxide (23c)

The compound was obtained from 13c by following the experimental conditions described for Method A: ¹H NMR (400 MHz, DMSO-d6): δ 1.83 (m, 2H), 2.63 (2H, t, J=7.8 Hz), 3.26 (q, 2H) 7.16-7.31 (m, 7H), 7.71 (1H, dd, J=8.7, 2.2 Hz), 7.76 (1H, d, J=2.2 Hz), 10.73 (br, 1H); ¹³C NMR (100 MHz, DMSO-d6): δ 30.95, 32.78, 114.97, 119.56, 124.61, 125.36, 126.28, 128.74, 128.79, 135.50, 135.55, 141.85, 151.49; HRMS (ESI) m/z calcd for C₁₆H₁₆BrN₃O₂S [M+Na]⁺: 416.00443 found: 416.00367.

### 3-((3-phenylpropyl)amino)-4H-benzo[e][1,2,4]thiadiazine 1,1-dioxide (23d)

The compound was obtained from 13d by following the experimental conditions described for Method A: ¹H NMR (400 MHz, DMSO-d6): δ 1.83 (m, 2H), 2.64 (2H, t, J=7.5 Hz), 3.24 (q, 2H) 7.17-7.31 (m, 8H), 7.54 (1H, t, J=8.3 Hz), 7.65 (1H, d, J=7.8 Hz), 10.59 (s, 1H); ¹³C NMR (100 MHz, DMSO-d6): δ 31.04, 32.80, 116.94, 123.10, 123.27, 124.09, 126.28, 128.75, 128.80, 132.78, 136.19, 141.89, 151.61; HRMS (ESI) m/z calcd for C₁₆H₁₇N₃O₂S [M+Na]⁺: 338.093919 found: 338.0938.

### 7-fluoro-3-((1-phenylethyl)amino)-4H-benzo[e][1,2,4]thiadiazine 1,1-dioxide (24a)

The compound was obtained from 13a by following the experimental conditions described for Method B: ¹H NMR (400 MHz, DMSO-d6): δ 1.48 (3H, d, J=7 Hz), 5.02 (m, 1H), 7.27 (m, 2H), 7.39 (m, 4H), 7.48 (m, 2H), 7.70 (br, 1H), 10.52 (s, 1H); ¹³C NMR (100 MHz, DMSO-d6): δ 32.91, 50.47, 109.48, 119.67, 120.54, 122.89, 126.41, 127.55, 128.91, 132.66, 143.84, 150.95, 156.85, 159.27; HRMS (ESI) m/z calcd for C₁₅H₁₄FN₃O₂S [M+Na]⁺: 342.068847 found: 342.0676.

### 6-chloro-3-((1-phenylethyl)amino)-4H-benzo[e][1,2,4]thiadiazine 1,1-dioxide (24b)

The compound was obtained from 13b by following the experimental conditions described for Method B: ¹H NMR (400 MHz, DMSO-d6): δ 1.48 (3H, d, J=7 Hz), 5.02 (m, 1H), 7.28 (m, 3H), 7.38 (m, 4H), 7.66 (1H, d, J=8.3 Hz), 7.84 (s, 1H), 10.58 (s, 1H); ¹³C NMR (100 MHz, DMSO-d6): δ 22.87, 50.48, 116.64, 120.80, 121.88, 124.34, 125.47, 126.43, 127.57, 128.92, 137.00, 137.63, 143.74, 150.68; HRMS (ESI) m/z calcd for C₁₅H₁₄ClN₃O₂S [M+Na]⁺: 358.039297 found: 358.0385.

### 7-bromo-3-((1-phenylethyl)amino)-4H-benzo[e][1,2,4]thiadiazine 1,1-dioxide (24c)

The compound was obtained from 13c by following the experimental conditions described for Method B: ¹H NMR (400 MHz, DMSO-d6): δ 1.49 (3H, d, J=7 Hz), 5.02 (m, 1H), 7.19 (1H, d, J=8.7 Hz), 7.26 (m, 1H), 7.38 (m, 4H), 7.71 (2H, dd, J=8.7, 2.2 Hz), 7.76 (1H, d, J=2.2 Hz), 10.57 (s, 1H); ¹³C NMR (100 MHz, DMSO-d6): δ 22.84, 50.50, 115.12, 119.69, 124.58, 125.38, 126.43, 127.14, 127.57, 128.91, 129.23, 135.31, 135.61, 143.73, 150.71; HRMS (ESI) m/z calcd for C₁₅H₁₄BrN₃O₂S [M+Na]⁺: 401.98878 found: 401.98855.

### 3-((1-phenylethyl)amino)-4H-benzo[e][1,2,4]thiadiazine 1,1-dioxide (24d)

The compound was obtained from 13d by following the experimental conditions described for Method B: ¹H NMR (400 MHz, DMSO-d6): δ 1.47 (3H, d, J=6.9 Hz), 5.03 (m, 1H), 7.19 (1H, d, J=8.2 Hz), 7.25 (m, 2H), 7.39 (m, 4H), 7.54 (2H, t, J=8.3 Hz), 7.58 (br, 1H), 7.67 (1H, d, J=7.6 Hz), 10.42 (s, 1H); ¹³C NMR (100 MHz, DMSO-d6): δ 22.96, 50.36, 117.06, 123.08, 123.28, 124.26, 126.41, 127.54, 128.92, 132.86, 135.98, 143.91, 150.82; HRMS (ESI) m/z calcd for C₁₅H₁₅N₃O₂S [M+Na]⁺: 324.078269 found: 324.0782.

### 7-fluoro-3-((4-(trifluoromethyl)benzyl)amino)-4H-benzo[e][1,2,4]thiadiazine 1,1-dioxide (25a)

The compound was obtained from 13a by following the experimental conditions described for Method A: ¹H NMR (400 MHz, DMSO-d6): δ 4.58 (2H, d, J=5.9 Hz), 7.30 (m, 1H), 7.45-7.56 (m, 4H), 7.71 (2H, d, J=8.2 Hz), 7.76 (br, 1H), 11.00 (br, 1H); ¹³C NMR (100 MHz, DMSO-d6): δ 43.94, 109.27, 119.52, 120.62, 123.41, 123.82, 125.69, 126.11, 128.18, 132.81, 144.05, 151.82, 156.88, 159.30; HRMS (ESI) m/z calcd for C₁₅H₁₁F4N₃O₂S [M+Na]⁺: 396.040581 found: 396.0388.

### 6-chloro-3-((4-(trifluoromethyl)benzyl)amino)-4H-benzo[e][1,2,4]thiadiazine 1,1-dioxide (25b)

The compound was obtained from 13b by following the experimental conditions described for Method A: ¹H NMR (400 MHz, DMSO-d6): δ 4.50 (2H, d, J=5.8 Hz), 7.30 (s, 1H), 7.32 (1H, d, J=2 Hz), 7.37 (2H, d, J=8.5 Hz), 7.45 (2H, d, J=8.7 Hz), 7.70 (1H, d, J=8.7 Hz), 7.86 (s, 1H), 10.90 (s, 1H); ¹³C NMR (100 MHz, DMSO-d6): δ 43.66, 116.65, 119.26, 121.50, 121.81, 121.87, 124.35, 125.45, 129.55, 137.01, 137.53, 138.48, 147.83, 151.48; HRMS (ESI) m/z calcd for C₁₅H₁₁ClF₃N₃O₂S [M+Na]⁺: 412.011031 found: 412.1504.

### 7-bromo-3-((4-(trifluoromethyl)benzyl)amino)-4H-benzo[e][1,2,4]thiadiazine 1,1-dioxide (25c)

The compound was obtained from 13c by following the experimental conditions described for Method A: ¹H NMR (400 MHz, DMSO-d6 δ 4.58 (2H, d, J=5.2 Hz), 7.22 (1H, d, J=8.7 Hz), 7.55 (2H, d, J=8.1 Hz), 7.73 (m, 3H), 7.78 (1H, d, J=2.2 Hz), 7.83 (br, 1H), 7.86, 11.04 (s, 1H); ¹³C NMR (100 MHz, DMSO-d6): δ 43.94, 115.16, 119.28, 123.41, 124.50, 125.41, 125.71, 126.11, 127.99, 128.21, 128.30, 135.51, 135.69, 143.97, 151.61; HRMS (ESI) m/z calcd for C₁₅H₁₁BrF₃N₃O₂S [M+Na]⁺: 455.960514 found: 455.95914.

### 7-fluoro-3-((2-methoxybenzyl)amino)-4H-benzo[e][1,2,4]thiadiazine 1,1-dioxide (26a)

The compound was obtained from 13a by following the experimental conditions described for Method A: ¹H NMR (400 MHz, DMSO-d6): δ 3.48 (s, 3H), 4.43 (2H, d, J=5.7 Hz), 6.93 (1H, t, J=7.3 Hz), 7.02 (1H, d, J=8.1 Hz), 7.28 (m, 3H), 7.46 (2H, t, J=8.7 Hz), 7.52 (1H, dd, J=7.5, 2.8 Hz), 10.78 (s, 1H); ¹³C NMR (100 MHz, DMSO-d6): δ 55.83, 109.51, 111.03, 119.47, 120.68, 123.79, 126.14, 128.38, 129.05, 132.78, 151.84, 156.83, 157.21, 159.25; HRMS (ESI) m/z calcd for C₁₅H₁₄FN₃O₃S [M+Na]⁺: 358.063762 found: 358.0608.

### 6-chloro-3-((2-methoxybenzyl)amino)-4H-benzo[e][1,2,4]thiadiazine 1,1-dioxide (26b)

The compound was obtained from 13b by following the experimental conditions described for Method A: ¹H NMR (400 MHz, DMSO-d6): δ 3.48 (s, 3H), 4.43 (2H, d, J=5.7 Hz), 6.93 (1H, t, J=7.3 Hz), 7.03 (1H, d, J=8.2 Hz), 7.28 (m, 4H), 7.66 (br, 1H), 7.68 (1H, d, J=8.3 Hz), 10.47 (br, 1H); ¹³C NMR (100 MHz, DMSO-d6): δ 55.86, 111.08, 116.57, 120.68, 121.84, 124.25, 125.43, 126.04, 128.44, 129.08, 136.99, 137.53, 151.55, 157.23; HRMS (ESI) m/z calcd for C₁₅H₁₄ClN₃O₃S [M+Na]⁺: 374.034212 found: 374.0336.

### 7-bromo-3-((2-methoxybenzyl)amino)-4H-benzo[e][1,2,4]thiadiazine 1,1-dioxide (26c)

The compound was obtained from 13c by following the experimental conditions described for Method A: ¹H NMR (400 MHz, DMSO-d6): δ 3.84 (s, 3H), 4.42 (2H, d, J=5.3 Hz), 6.93 (1H, t, J=7.3 Hz), 7.03 (1H, d, J=8.2 Hz), 7.18 (1H, d, J=8.5 Hz), 7.22 (1H, d, J=7.2 Hz), 7.28 (1H, t, J=7.7 Hz) 7.46 (br, 1H), 7.72 (1H, dd, J=8.7, 2 Hz), 7.77 (1H, d, J=2 Hz), 10.69 (br, 1H); ¹³C NMR (100 MHz, DMSO-d6): δ 55.86, 111.08, 115.00, 119.60, 120.69, 124.51, 125.40, 126.06, 128.42, 129.08, 135.61, 151.61, 157.22; HRMS (ESI) m/z calcd for C₁₅H₁₄BrN₃O₃S [M+Na]⁺: 417.983695 found: 417.98163.

### 7-fluoro-3-((4-methylbenzyl)amino)-4H-benzo[e][1,2,4]thiadiazine 1,1-dioxide (27a)

The compound was obtained from 13a by following the experimental conditions described for Method A: ¹H NMR (400 MHz, DMSO-d6): δ 2.28 (s, 3H), 4.44 (2H, d, J=5.7 Hz), 7.16 (2H, d, J=7.8 Hz), 7.22 (2H, d, J=7.8 Hz), 7.28 (m, 1H), 7.46 (m, 1H), 7.52 (1H, dd, J=7.5, 2.8 Hz), 7.64 (br, 1H), 10.81 (s, 1H); ¹³C NMR (100 MHz, DMSO-d6): δ 21.13, 44.10, 109.49, 119.54, 120.53, 120.77, 123.89, 127.66, 129.40, 132.84, 135.86, 136.68, 151.75, 156.83, 157.21, 159.25; HRMS (ESI) m/z calcd for C₁₅H₁₄FN₃O₂S [M+Na]⁺: 342.068847 found: 342.0672.

### 6-chloro-3-((4-methylbenzyl)amino)-4H-benzo[e][1,2,4]thiadiazine 1,1-dioxide (27b)

The compound was obtained from 13b by following the experimental conditions described for Method A: ¹H NMR (400 MHz, DMSO-d6): δ 2.28 (s, 3H), 4.41 (2H, d, J=4.2 Hz), 7.17 (2H, d, J=7.9 Hz), 7.22 (2H, d, J=7.9 Hz), 7.28 (s, 1H), 7.30 (s, 1H), 7.68 (1H, d, J=8.3 Hz), 7.76 (br, 1H), 10.64 (br, 1H); ¹³C NMR (100 MHz, DMSO-d6): δ 21.14, 44.10, 116.66, 121.89, 124.23, 125.42, 127.70, 129.40, 135.76, 136.70, 136.96, 137.65, 151.49; HRMS (ESI) m/z calcd for C₁₅H₁₄ClN₃O₂S [M+Na]⁺: 358.039297 found: 358.0388.

### 7-bromo-3-((4-methylbenzyl)amino)-4H-benzo[e][1,2,4]thiadiazine 1,1-dioxide (27c)

The compound was obtained from 13c by following the experimental conditions described for Method A: ¹H NMR (400 MHz, DMSO-d6): δ 2.28 (s, 3H), 4.43 (2H, d, J=4.6 Hz), 7.15-7.23 (m, 5H), 7.67 (br, 1H), 7.72 (1H, dd, J=8.7, 2.2 Hz), 7.77 (1H, d, J=2.2 Hz), 10.83 (s, 1H); ¹³C NMR (100 MHz, DMSO-d6): δ 21.48, 44.10, 116.67, 121.90, 124.23, 125.42, 127.70, 129.40, 135.77, 136.70, 136.95, 137.65, 151.48; HRMS (ESI) m/z calcd for C₁₅H₁₄BrN₃O₂S [M+Na]⁺: 401.98878 found: 401.98857.

### 6-chloro-3-((3-methoxybenzyl)amino)-4H-benzo[e][1,2,4]thiadiazine 1,1-dioxide (28b)

The compound was obtained from 13b by following the experimental conditions described for Method A: ¹H NMR (400 MHz, DMSO-d6): δ 3.74 (s, 3H), 4.46 (2H, d, J=5.3 Hz), 6.85 (1H, dd, J=8, 2 Hz), 6.94 (m, 2H), 7.27 (1H, d, J=7.8 Hz), 7.29 (2H, d, J=7 Hz), 7.70 (1H, d, J=8.8 Hz), 7.79 (br, 1H), 10.79 (s, 1H); ¹³C NMR (100 MHz, DMSO-d6): δ 44.29, 55.45, 112.99, 113.38, 116.66, 119.81, 121.78, 124.30, 125.43, 129.95, 137.03, 137.54, 140.42, 151.47, 159.81; HRMS (ESI) m/z calcd for C₁₅H₁₄ClN₃O₃S [M+Na]⁺: 374.034212 found: 374.0335.

### 7-bromo-3-((3-methoxybenzyl)amino)-4H-benzo[e][1,2,4]thiadiazine 1,1-dioxide (28c)

The compound was obtained from 13c by following the experimental conditions described for Method A: ¹H NMR (400 MHz, DMSO-d6): δ 3.74 (s, 3H), 4.44 (s, 2H), 6.85 (1H, d, J=7.9 Hz), 6.92 (2H, d, J=10.5 Hz), 7.20 (1H, d, J=8.6 Hz), 7.26 (1H, t, J=7.8 Hz), 7.72 (2H, dd, J=8.7, 2 Hz), 7.77 (1H, d, J=2 Hz), 10.79 (br, 1H); ¹³C NMR (100 MHz, DMSO-d6): δ 44.28, 55.47, 113.01, 113.36, 115.05, 119.68, 119.79, 124.57, 125.39, 129.95, 135.62, 140.45, 151.59, 159.81; HRMS (ESI) m/z calcd for C₁₅H₁₄BrN₃O₃S [M+Na]⁺: 417.983695 found: 417.98193.

### 6-chloro-3-((2,4-difluorobenzyl)amino)-4H-benzo[e][1,2,4]thiadiazine 1,1-dioxide (29b)

The compound was obtained from 13b by following the experimental conditions described for Method A: ¹H NMR (400 MHz, DMSO-d6): δ 4.47 (2H, d, J=5.6 Hz), 7.10 (1H, t, J=8.5 Hz), 7.24-7.32 (m, 3H), 7.45 (q, 1H), 7.69 (1H, d, J=8.5 Hz), 7.79 (br, 1H), 10.84 (s, 1H); ¹³C NMR (100 MHz, DMSO-d6): δ 38.08, 104.26, 111.77, 116.67, 121.78, 124.40, 125.47, 131.23, 137.04, 137.44, 151.38, 159.34, 160.77, 161.68, 163.33; HRMS (ESI) m/z calcd for C₁₅H₁₀ClF₂N₃O₂S [M+Na]⁺: 380.004803 found: 380.0051.

### 7-bromo-3-((2,4-difluorobenzyl)amino)-4H-benzo[e][1,2,4]thiadiazine 1,1-dioxide (29c)

The compound was obtained from 13c by following the experimental conditions described for Method A: ¹H NMR (400 MHz, DMSO-d6): δ 4.48 (2H, d, J=5.5 Hz), 7.10 (1H, t, J=8.5 Hz), 7.20 (1H, d, J=8.7 Hz), 7.27 (m, 1H), 7.45 (q, 1H), 7.70 (br, 1H), 7.73 (1H, dd, J=8.7, 2.2 Hz), 7.78 (1H, d, J=2.2 Hz), 10.93 s, 1H); ¹³C NMR (100 MHz, DMSO-d6): δ 38.09, 104.27, 111.99, 115.20, 119.66, 122.06, 124.45, 125.42, 131.20, 135.39, 135.69, 151.43, 159.20, 160.76, 161.79, 163.32; HRMS (ESI) m/z calcd for C₁₅H₁₀BrF₂N₃O₂S [M+Na]⁺: 423.954286 found: 423.95383.

### 7-Fluoro-3-((4-(trifluoromethoxy)benzyl)amino)-4H-benzo[e][1,2,4]thiadiazine 1,1-dioxide (30a)

The compound was obtained from 13a by following the experimental conditions described for Method A: ¹H NMR (400 MHz, DMSO-d6): δ 4.51 (2H, d, J=5.7 Hz), 7.29 (q, 1H), 7.37 (2H, d, J=8.2 Hz), 7.44-7.52 (m, 4H), 7.73 (br, 1H), 10.91 (s, 1H); ¹³C NMR (100 MHz, DMSO-d6): δ 43.64, 109.51, 119.26, 119.59, 120.60, 121.51, 123.85, 129.50, 132.80, 138.63, 147.80, 151.77, 156.87, 159.29; HRMS (ESI) m/z calcd for C₁₅H₁₁F4N₃O₃S [M+Na]⁺: 412.035496 found: 412.0304.

### 6-chloro-3-((4-(trifluoromethoxy)benzyl)amino)-4H-benzo[e][1,2,4]thiadiazine 1,1-dioxide (30b)

The compound was obtained from 13b by following the experimental conditions described for Method A: ¹H NMR (400 MHz, DMSO-d6): δ 4.51 (2H, d, J=5.7 Hz), 7.30 (2H, d, J=7.8 Hz), 7.37 (2H, d, J=8.6 Hz), 7.45 (2H, d, J=8.6 Hz), 7.69 (1H, d, J=8.6 Hz), 7.85 (br, 1H), 10.90 (s, 1H); ¹³C NMR (100 MHz, DMSO-d6): δ 43.65, 116.66, 119.26, 121.51, 121.81, 121.85, 124.35, 125.45, 129.54, 137.03, 137.52, 138.49, 147.84, 151.47; HRMS (ESI) m/z calcd for C₁₅H₁₁ClF₃N₃O₃S [M+Na]⁺: 428.005946 found: 428.0062.

### 7-bromo-3-((4-(trifluoromethoxy)benzyl)amino)-4H-benzo[e][1,2,4]thiadiazine 1,1-dioxide (30c)

The compound was obtained from 13c by following the experimental conditions described for Method A: ¹H NMR (400 MHz, DMSO-d6): δ 4.50 (2H, d, J=5 Hz), 7.21 (1H, d, J=8.7 Hz), 7.36 (2H, d, J=8.2 Hz), 7.45 (2H, d, J=8.5 Hz), 7.74 (1H, d, J=8.6.2 Hz), 7.78 (2H, d, J=2 Hz), 10.84 (s, 1H); ¹³C NMR (100 MHz, DMSO-d6): δ 43.65, 115.10, 116.72, 119.26, 119.68, 121.49, 121.81, 124.54, 125.40, 129.53, 135.55, 135.64, 138.52, 147.82, 151.58; HRMS (ESI) m/z calcd for C₁₅H₁₁BrF₃N₃O₃S [M+Na]⁺: 471.955429 found: 471.95405.

### 3-((4-(trifluoromethoxy)benzyl)amino)-4H-benzo[e][1,2,4]thiadiazine 1,1-dioxide (30d)

The compound was obtained from 13d by following the experimental conditions described for Method A: ¹H NMR (400 MHz, DMSO-d6): δ 4.52 (2H, d, J=5.8 Hz), 7.22 (1H, d, J=8.2 Hz), 7.26 (1H, t, J=7.6 Hz), 7.37 (2H, d, J=8.2 Hz), 7.46 (2H, d, J=8.6 Hz), 7.56 (1H, t, J=8.2 Hz), 7.61 (br, 1H), 7.67 (1H, d, J=7.2 Hz), 10.83 (s, 1H); ¹³C NMR (100 MHz, DMSO-d6): δ 43.55, 117.04, 119.27, 121.50, 121.81, 123.05, 123.30, 124.27, 129.50, 132.89, 136.12, 138.71, 147.79, 151.62; HRMS (ESI) m/z calcd for C₁₅H₁₂F₃N₃O₃S [M+Na]⁺: 394.044918 found: 394.0439.

### 3-((4-aminobenzyl)amino)-7-bromo-4H-benzo[e][1,2,4]thiadiazine 1,1-dioxide (31c)

The compound was obtained from 13c by following the experimental conditions described for Method B: ¹H NMR (400 MHz, DMSO-d6): δ 4.26 (s, 2H), 5.03 (s, 2H) 6.52 (s, 2H), 7.00 (s, 2H), 7.16 (s, 1H), 7.46 (s, 1H), 7.76 (d, 2H), 10.15 (s, 1H); ¹³C NMR (100 MHz, DMSO-d6): δ 44.28, 114.02, 114.17, 114.73, 119.76, 124.63, 125.35, 128.93, 130.36, 135.49, 135.89, 148.39, 151.56; HRMS (ESI) m/z calcd for C₁₄H₁₃BrN₄O₂S [M+Na]⁺: 402.984029 found: 402.98192.

### 3-(allylamino)-7-fluoro-4H-benzo[e][1,2,4]thiadiazine 1,1-dioxide (32a)

The compound was obtained from 13a by following the experimental conditions described for Method A: ¹H NMR (400 MHz, DMSO-d6): δ 3.88 (m, 3H), 5.12-5.23 (m, 2H), 5.88 (m, 1H), 7.28 (q, 1H), 7.36 (br, 1H), 7.43-7-57 (m, 2H), 10.75 (s, 1H); ¹³C NMR (100 MHz, DMSO-d6): δ 43.15, 109.47, 116.23, 119.52, 120.75, 123.86, 132.80, 134.92, 151.65, 156.82, 159.24; HRMS (ESI) m/z calcd for C₁₀H₁₀FN₃O₂S [M+Na]⁺: 278.037547 found: 278.0351.

### 3-(allylamino)-6-chloro-4H-benzo[e][1,2,4]thiadiazine 1,1-dioxide (32b)

The compound was obtained from 13b by following the experimental conditions described for Method A: ¹H NMR (400 MHz, DMSO-d6): δ 3.88 (m, 3H), 5.15 (m, 1H), 5.29 (m, 1H), 5.88 (m, 1H), 7.29 (s, 1H), 7.31 (1H, d, J=8.7 Hz), 7.50 (br, 1H), 7.68 (d, 1H), 10.74 (br, 1H); ¹³C NMR (100 MHz, DMSO-d6): δ 43.16, 116.32, 116.61, 121.85, 124.28, 125.42, 134.81, 136.69, 137.53, 151.33; HRMS (ESI) m/z calcd for C₁₀H₁₀ClN₃O₂S [M+Na]⁺: 294.007997 found: 294.0068.

### 3-(allylamino)-4H-benzo[e][1,2,4]thiadiazine 1,1-dioxide (32d)

The compound was obtained from 13d by following the experimental conditions described for Method A: ¹H NMR (400 MHz, DMSO-d6): δ 3.88 (m, 3H), 5.12-5.23 (m, 2H), 5.88 (q, 1H), 7.21 (1H, d, J=8.2 Hz), 7.25 (2H, t, J=8.3 Hz), 7.55 (1H, t, J=8.3 Hz), 7.65 (1H, dd, J=7.8, 2 Hz), 10.64 (s, 1H); ¹³C NMR (100 MHz, DMSO-d6): δ 43.05, 116.19, 116.98, 123.05, 123.28, 124.19, 132.82, 135.01, 136.13, 151.49; HRMS (ESI) m/z calcd for C₁₀H₁₁N₃O₂S [M+Na]⁺: 260.046969 found: 260.0338.

### 7-bromo-3-((4-fluorophenethyl)amino)-4H-benzo[e][1,2,4]thiadiazine 1,1-dioxide (33c)

The compound was obtained from 13c by following the experimental conditions described for Method A: ¹H NMR (400 MHz, DMSO-d6): δ 2.84 (2H, t, J=7.2 Hz), 3.47 (q, 2H), 7.14 (m, 4H), 7.29 (m, 2H), 7.73 (1H, dd, J=8.7, 2.2 Hz), 7.77 (1H, d, J=2.2 Hz), 10.78 (br, 1H); ¹³C NMR (100 MHz, DMSO-d6): δ 34.15, 42.41, 115.01, 115.46, 115.66, 119.51, 124.53, 125.39, 130.91, 130.99, 135.44, 135.58, 151.46, 160.19, 162.59; HRMS (ESI) m/z calcd for C₁₅H₁₃BrFN₃O₂S [M+Na]⁺: 419.979358 found: 419.97927.

### 7-bromo-3-((4-methoxyphenethyl)amino)-4H-benzo[e][1,2,4]thiadiazine 1,1-dioxide (34c)

The compound was obtained from 13c by following the experimental conditions described for Method A: ¹H NMR (400 MHz, DMSO-d6): δ 2.78 (2H, t, J=7.2 Hz), 3.43 (q, 2H), 3.72 (s, 3H), 6.89 (2H, d, J=8.6 Hz), 7.14 (br, 1H), 7.16 (3H, d, J=8.6 Hz), 7.71 (1H, dd, J=8.7, 2.2 Hz), 7.77 (1H, d, J=2.2 Hz), 10.75 (br, 1H); ¹³C NMR (100 MHz, DMSO-d6): δ 34.13, 42.66, 55.45, 114.42, 115.00, 119.52, 124.53, 125.38, 130.12, 130.06, 135.44, 135.58, 151.42, 158.27; HRMS (ESI) m/z calcd for C₁₆H₁₆BrN₃O₃S [M+Na]⁺: 431.999345 found: 431.99962.

### 7-bromo-3-((2-(trifluoromethyl)benzyl)amino)-4H-benzo[e][1,2,4]thiadiazine 1,1-dioxide (35c)

The compound was obtained from 13c by following the experimental conditions described for Method A: ¹H NMR (400 MHz, DMSO-d6): δ 4.67 (2H, d, J=5.4 Hz), 7.22 (1H, d, J=8.7 Hz), 7.54 (m, 1H), 7.67-7.79 (m, 5H), 11.09 (br, 1H); ¹³C NMR (100 MHz, DMSO-d6): δ 41.12, 114.22, 119.71, 123.53, 124.44, 125.45, 126.37, 128.15, 128.85, 133.72, 135.45, 135.73, 137.07, 151.61; HRMS (ESI) m/z calcd for C₁₅H₁₁₂BrF₃N₃O₂S [M+Na]⁺: 455.960514 found: 455.95883.

### 3-((2-(1H-indol-3-yl)ethyl)amino)-7-fluoro-4H-benzo[e][1,2,4]thiadiazine 1,1-dioxide (36a)

A mixture of 7-fluoro-3-methylsulfanyl-4H-1,2,4-benzothiadiazine 1,1-dioxide (13a) (0.25 g, 1.02 mmol) and tryptamine (0.19 g, 1.21 mmol) was dissolved in 1,4-dioxane (10 mL) and refluxed for 72 h. The reaction was allowed to cool and the solvent and excess amine removed in vacuo and the resulting residue dissolved in an aqueous 2% w/v solution of sodium hydroxide (7 mL). This solution was treated with charcoal and was filtered. The filtrate was adjusted to pH 3-4 using 6N HCl. The precipitated compound was collected by filtration, washed with water and air dried. The dried compound was suspended in an aqueous solution of sodium bicarbonate NaHCO₃ (1 g/40 mL). The alkaline solution was treated with charcoal and filtered, and the filtrate was adjusted to pH 4-5 with 6N HCl. The precipitate was collected by filtration, washed twice with water, and air dried.: ¹H NMR (400 MHz, DMSO-d6): δ 2.97 (2H, t, J=7.2 Hz), 3.56 (q, 2H), 6.99 (1H, t, J=7.7 Hz), 7.09 (2H, t, J=7.8 Hz), 7.22 (m, 2H), 7.35 (1H, d, J=8.1 Hz), 7.45 (1H, t, J=8.7 Hz), 7.53 (1H, dd, J=7.5, 2.8 Hz), 7.59 (1H, d, J=7.8 Hz), 10.72 (br, 1H), 10.90 (br, 1H); ¹³C NMR (100 MHz, DMSO-d6): δ 25.21, 41.70, 109.50, 111.53, 111.87, 118.78, 119.37, 120.73, 121.49, 123.37, 123.84, 127.59, 132.85, 136.74, 151.73, 156.77, 159.18; HRMS (ESI) m/z calcd for C₁₇H₁₅FN₄O₂S [M+Na]⁺: 381.079746 found: 381.0749.

### 3-((2-(1H-indol-3-yl)ethyl)amino)-6-chloro-4H-benzo[e][1,2,4]thiadiazine 1,1-dioxide (36b)

The compound was obtained from 13b by following the experimental conditions described for 27a: ¹H NMR (400 MHz, DMSO-d6): δ 2.97 (2H, t, J=7.2 Hz), 3.55 (q, 2H), 6.99 (1H, t, J=7.9 Hz), 7.08 (1H, t, J=7.9 Hz), 7.22 (1H, d, J=2.5 Hz), 7.23 (br, 1H), 7.30 (2H, dd, J=8.4, 2 Hz), 7.34 (1H, d, J=8.1 Hz), 7.61 (1H, d, J=7.8 Hz), 768 (1H, d, J=8.4 Hz), 10.71 (br, 1H), 10.90 (br, 1H); ¹³C NMR (100 MHz, DMSO-d6): δ 25.15, 41.71, 111.51, 111.87, 116.47, 118.78, 121.49, 123.38, 124.18, 125.44, 126.53, 127.59, 136.74, 136.93, 137.55, 151.43, 161.92; HRMS (ESI) m/z calcd for C₁₇H₁₅ClN₄O₂S [M+Na]⁺: 397.050196 found: 397.0498.

### 3-((4-chlorophenethyl)amino)-7-fluoro-4H-benzo[e][1,2,4]thiadiazine 1,1-dioxide (37a)

The compound was obtained from 13a by following the experimental conditions described for Method A: ¹H NMR (400 MHz, DMSO-d6): δ 2.85 (2H, t, J=7.1 Hz), 3.47 (q, 2H), 7.14 (br, 1H), 7.24 (m, 1H), 7.30 (1H, d, J=8.3 Hz), 7.36 (2H, d, J=8.3 Hz), 7.45 (1H, t, J=8.7 Hz), 7.51 (1H, dd, J=7.5, 2.8 Hz), 10.70 (s, 1H); ¹³C NMR (100 MHz, DMSO-d6): δ 34.32, 42.18, 109.48, 119.45, 120.74, 128.79, 131.08, 131.39, 131.60, 132.78, 138.36, 151.67, 159.22, 16.91; HRMS (ESI) m/z calcd for C₁₅H₁₃ClFN₃O₂S [M+Na]⁺: 376.029875 found: 376.0270.

### 7-fluoro-3-((4-((trifluoromethyl)thio)benzyl)amino)-4H-benzo[e][1,2,4]thiadiazine 1,1-dioxide (38a)

The compound was obtained from 13a by following the experimental conditions described for Method A: ¹H NMR (400 MHz, DMSO-d6): δ 4.57 (2H, d, J=5.9 Hz), 7.30 (m, 1H), 7.44-7.53 (m, 4H), 7.7 (2H, d, J=8.1 Hz), 7.77 (br, 1H), 11.05 (s, 1H); ¹³C NMR (100 MHz, DMSO-d6): δ 43.87, 109.51, 119.50, 120.84, 121.77, 123.83, 128.54, 129.00, 131.60, 132.82, 136.79, 143.08, 151.86, 156.88, 159.30; HRMS (ESI) m/z calcd for C₁₅H₁₁F₄N₃O₂S₂ [M+Na]⁺: 428.012653 found: 428.0131.

### 7-fluoro-3-((4-methoxyphenethyl)amino)-4H-benzo[e][1,2,4]thiadiazine 1,1-dioxide (39a)

The compound was obtained from 13a by following the experimental conditions described for Method A: ¹H NMR (400 MHz, DMSO-d6): δ 2.79 (2H, t, J=7.2 Hz), 3.44 (q, 2H), 3.72 (s, 3H), 6.88 (2H, d, J=8.6 Hz), 7.10 (br, 1H), 7.16 (2H, d, J=8.5 Hz), 7.24 (br, 1H), 7.44 (1H, t, J=8.7 Hz), 7.52 (1H, dd, J=7.5, 2.8 Hz) 10.73 (br, 1H); ¹³C NMR (100 MHz, DMSO-d6): δ 34.20, 42.66, 55.42, 109.47, 114.30, 119.40, 120.47, 120.71, 123.75, 130.12, 131.09, 132.82, 151.70, 156.78, 158.26, 159.20; HRMS (ESI) m/z calcd for C₁₆H₁₆FN₃O₃S [M+Na]⁺: 372.0794 found: 372.0720.

### 6-chloro-3-((3-methoxyphenethyl)amino)-4H-benzo[e][1,2,4]thiadiazine 1,1-dioxide (40b)

The compound was obtained from 13b by following the experimental conditions described for Method A: ¹H NMR (400 MHz, DMSO-d6): δ 2.83 (2H, t, J=7.1 Hz), 3.48 (q, 2H), 3.74 (s, 3H), 6.80 (1H, d, J=8.2 Hz), 6.87 (m, 2H), 7.23 (m, 3H), 7.29 (1H, dd, J=8.4, 2 Hz), 7.68 (1H, d, J=8.4 Hz), 10.67 (br, 1H); ¹³C NMR (100 MHz, DMSO-d6): δ 34.98, 42.29, 55.36, 112.19, 114.78, 116.53 121.37, 121.86, 124.23, 125.42, 129.91, 136.94, 137.51, 140.86, 151.38, 159.79; HRMS (ESI) m/z calcd for C₁₆H₁₆ClN₃O₃S [M+Na]⁺: 388.0498 found: 388.0542.

### 7-fluoro-3-((3-(trifluoromethyl)phenethyl)amino)-4H-benzo[e][1,2,4]thiadiazine 1,1-dioxide (41a)

The compound was obtained from 13a by following the experimental conditions described for Method A: ¹H NMR (400 MHz, DMSO-d6): δ 2.97 (2H, t, J=7 Hz), 3.52 (q, 2H), 7.25 (br, 2H), 7.45 (1H, t, J=8.7 Hz), 7.50 (1H, dd, J=7.5, 2.8 Hz), 7.57 (q, 3H), 7.63 (s, 1H), 10.71 (br, 1H); ¹³C NMR (100 MHz, DMSO-d6): δ 34.69, 42.06, 109.45, 119.45, 120.48, 120.72, 123.51, 123.83, 125.72, 129.84, 132.78, 133.40, 140.81, 151.74, 156.87, 159.24; HRMS (ESI) m/z calcd for C₁₆H₁₃F₄N₃O₂S [M+Na]⁺: 410.0562 found: 410.0497.

### 7-fluoro-4-methyl-3-(methylthio)-4H-benzo[e][1,2,4]thiadiazine 1,1-dioxide (42a)

To a solution of 7-fluoro-3-methylsulfanyl-4H-1,2,4-benzothiadiazine1,1-dioxide (13a) (1 g, 3.8 mmol) in acetonitrile/DMF, 4:1 (15 mL) at room temperature was added K₂CO₃ (0.48 g, 3.45 mmol) and methyl iodide (1 mL, 6.9 mmol). The mixture was stirred for 10 h and the solvent was removed in vacuo. The solid residue was taken up in water (20 mL). The resulting aqueous suspension was adjusted to pH 2 by means of formic acid, and the precipitate was collected by filtration and washed with water. The crude compound was recrystallized in methanol/water to provide the title compound: ¹H NMR (400 MHz, DMSO-d6): δ 2.55 (s, 3H), 3.71 (s, 3H), 7.68 (m, 2H), 7.73 (m, 1H); ¹³C NMR (100 MHz, DMSO-d6): δ 16.09, 36.41, 110.29, 120.66, 121.48, 125.01, 135.26, 158.17, 160.63, 166.35; HRMS (ESI) m/z calcd for C₉H₉FN₂O₂S₂ [M+Na]⁺: 282.99872 found: 282.9954.

### 4-methyl-3-(methylthio)-4H-benzo[e][1,2,4]thiadiazine 1,1-dioxide (42d)

The compound was obtained from 13d by following the experimental conditions described for 42a: ¹H NMR (400 MHz, DMSO-d6): δ 2.54 (s, 3H), 3.70 (s, 3H), 7.60 (m, 2H), 7.79 (1H, t, J=8.6 Hz), 7.86 (1H, dd, J=7.8, 2 Hz)); ¹³C NMR (100 MHz, DMSO-d6): δ 15.98, 36.13, 117.45, 123.96, 126.74, 133.82, 138.43, 166.09 HRMS (ESI) m/z calcd for C₈H₁₀N₂O₂S₂[M+Na]⁺: 265.008142 found: 264.992.

### 3-(isopropylamino)-4-methyl-4H-benzo[e][1,2,4]thiadiazine 1,1-dioxide (43d)

To a solution of 4-methyl-3-methylsulfanyl-4H-1,2,4-benzothiadiazine 1,1-dioxide (42d, 0.2 g, 0.79 mmol) in 1,4-dioxane (3 mL) in a sealed vessel was added isopropylamine (0.2 mL, 3.16 mmol) and the mixture heated for 24 h at 130° C. The excess solvent and amine were removed by distillation under reduced pressure, and the residue was suspended in water (15 mL). The mixture was stirred for 1 h at room temperature, the resultant precipitate was collected by filtration, washed twice with water, and recrystallized from methanol/water to yield the title compound: ¹H NMR (400 MHz, DMSO-d6): δ 1.21 (6H, d, J=6.5 Hz), 3.46 (s, 3H), 4.05 (q, 1H), 7.36 (1H, t, J=7.5 Hz), 7.45 (2H, d, J=8.3 Hz), 7.65 (1H, t, J=8.5 Hz), 7.70 (1H, dd, J=7.8, 2 Hz)¹³C NMR (100 MHz, DMSO-d6): δ 22.53, 35.09, 44.80, 66.80, 117.22, 122.76, 124.42, 126.45, 132.77, 139.26, 153.37; HRMS (ESI) m/z calcd for C₁H₁₅N₃O₂S [M+Na]⁺: 276.0552 found: 276.0782.

### 7-Chloro-3-oxo-3,4-dihydro-2H-1,2,4-benzothiadiazine1,1-dioxide (51e)

A solution of chlorosulfonyl isocyanate (3.3 mL, 37.6 mmol) in nitromethane (50 mL) was mixed in a closed dried vessel under nitrogen pressure and cooled to −10° C. (ice and salt bath). The 4-chloroaniline (50e. 4 g, 31.4 mmol) was added dropwise. The contents were vigorously stirred for 15 mins followed by the addition of anhydrous aluminum chloride (5.44 g, 40.8 mmol) and the mixture was refluxed for 1 h. The hot solution was poured onto ice (200 g) and stirred for an additional 30 mins until all ice is melted and the resulting precipitate was collected by filtration and washed with water. The crude solid was treated with an aqueous solution of sodium bicarbonate (5 g/100 mL) followed by heating until the solid precipitate was dissolved. The solution was treated with charcoal and was filtered, the filtrate solution was adjusted to pH 1 using 12N HCl. The resulting precipitate was filtered, washed with water, and air dried: ¹H NMR (400 MHz, DMSO-d₆): δ=7.29 (1H, d, J=7.3 Hz), 7.7 (1H, d, J=7.7), 7.83 (s, 1H), 11.45 ppm (s, 1H). ¹³C NMR (100 MHz, DMSO-d₆): δ=119.62, 122.09, 124.05, 127.69, 134.27, 134.53, 151.57 ppm. HRMS (ESI): m/z calcd for C₇H₅ClN₂O₃S [M+Na]⁺: 254.9607, found: 254.9648.

### 7-Chloro-3-thioxo-3,4-dihydro-2H-1,2,4-benzothiadiazine 1,1-dioxide (52e)

A suspension of 7-chloro-3-oxo-3,4-dihydro-2H-1,2,4-benzothiadiazine1,1-dioxide (51e, 4.5 g, 19.34 mmol) and phosphorus pentasulfide (8.4 g, 19.34 mmol) was dissolved in anhydrous pyridine (50 mL) and refluxed under nitrogen pressure overnight. The reaction was allowed to cool, and the solvent removed in vacuo, the crude product was dissolved in an aqueous solution of sodium hydroxide (NaOH) (5 g/100 mL). This solution was treated with charcoal and was filtered. The filtrate was acidified to pH 1 using 12N HCl. The precipitated compound was collected by filtration, washed with water, and was allowed to air dry. The dried compound was suspended in an aqueous solution of sodium bicarbonate (NaHCO₃) (10 g/200 mL) and heated until the solid was dissolved. This solution was treated with charcoal and filtered. The filtrate was adjusted to pH 1 using 12N HCl, and the precipitate was collected by filtration, washed with water, and air dried: ¹H NMR (400 MHz, DMSO-d₆): δ=7.25 (1H, d, J=7.2), 7.49 (1H, dd, J=7.5, 2.8 Hz), 7.55 (s, 1H), 11.35 ppm (br, 1H). ¹³C NMR (100 MHz, DMSO-d₆): δ=109.88, 121.31, 122.43, 124.51, 132.66, 158.16, 160.83 ppm. HRMS (ESI): m/z calcd for C₇H₅ClN₂O₂S₂ [M+Na]⁺: 270.9379, found: 270.9318.

### 7-Chloro-3-methylsulfanyl-4H-1,2,4-benzothiadiazine 1,1-dioxide (53e)

7-Chloro-3-thioxo-3,4dihydro-2H-1,2,4-benzothiadiazine 1,1-dioxide (52e, 4.0 g, 15.22 mmol) was suspended in a 1:1 hydromethanolic solution of sodium bicarbonate (5 g/200 mL). Methyl iodide was added (2 mL, 30.44 mmol) and the solution was stirred for 1 h. The resulting suspension was adjusted to pH 5 using 6N HCl. The suspension was concentrated under reduced pressure, and the precipitate was collected by filtration, washed with water, and air dried: ¹H NMR (400 MHz, DMSO-d₆): δ=2.53 (s, 2H), 7.31 (1H, dd, J=8.8, 2.2 Hz), 7.72 (1H, dd, J=8.8, 2.3 Hz), 7.84 (1H, d, J=2.3 Hz), 12.66 ppm (s, 1H). ¹³C NMR (100 MHz, DMSO-d₆): δ=13.87, 119.72, 123.16, 123.20, 129.68, 133.88, 133.94, 161.79 ppm. HRMS (ESI): m/z calcd for C₈H₇ClN₂O₂S₂ [M+Na]⁺: 284.9535, found: 284.9486.

### 7-Chloro-3-((4-fluorobenzyl)amino)-4H-benzo[e][1,2,4]thiadiazine 1,1-dioxide (54e)

7-Chloro-3-methylsulfanyl-4H-1,2,4-benzothiadiazine 1,1-dioxide (53e, 0.25 g, 1 mmol) and 4-fluorobenzylamine (0.23 mL, 1.9 mmol) were dissolved in 1,4-dioxane (8 mL) in a sealed vessel and heated for 24 h at 140° C. The solvent and the excess amine were removed in vacuo, and the residue was dissolved in an aqueous 2% w/v solution of NaOH (6 mL). This solution was treated with charcoal and was filtered. The filtrate was adjusted to pH 1 using 6N HCl. The precipitated compound was collected by filtration, washed with water and air dried. The dried compound was suspended in an aqueous solution of sodium bicarbonate NaHCO₃ (1 g/40 mL). The alkaline solution was treated with charcoal and filtered; the filtrate was adjusted to pH 4-5 with 6N HCl. The precipitate was collected by filtration, washed twice with water, and air dried: ¹H NMR (400 MHz, DMSO-d₆): δ=4.46 (s, 2H), 7.18 (2H, t, J=8.8 Hz), 7.26 (1H, d, J=8.8 Hz), 7.39 (q, 2H), 7.59 (1H, dd, J=8.7, 2.2 Hz), 7.67 (1H, d, J=2.2 Hz), 7.76 (br, 1H), 10.94 ppm (br, 1H). ¹³C NMR (100 MHz, DMSO-d₆): δ=43.64, 115.49, 115.70, 119.42, 122.60, 124.21, 127.54, 129.71, 129.79, 132.87, 135.17, 151.73, 160.59, 163.01 ppm. HRMS (ESI): m/z calcd for C₁₄H₁₁ClFN₃O₂S [M+Na]⁺: 362.0142, found: 362.0160.

### 7-Chloro-3-(phenethylamino)-4H-benzo[e][1,2,4]thiadiazine 1,1-dioxide (55e)

The compound was obtained from 53e by following the experimental conditions described for 54e: ¹H NMR (400 MHz, DMSO-d₆): δ=2.85 (2H, t, J=7.2 Hz), 3.48 (s, 2H), 7.20-7.34 (m, 7H), 7.56 (1H, dd, J=8.7 Hz), 7.67 (s 1H), 10.91 ppm (br, 1H). ¹³C NMR (100 MHz, DMSO-d₆): δ=35.05, 42.43, 119.30, 121.61, 124.19, 125.83, 126.82, 127.56, 128.90, 129.12, 129.15, 132.84, 135.15, 139.28, 151.52 ppm. HRMS (ESI): m/z calcd for C₁₅H₁₄ClN₃O₂S [M+Na]⁺: 358.0392, found: 358.0379.

### 7-Chloro-3-((4-methoxybenzyl)amino)-4H-benzo[e][1,2,4]thiadiazine 1,1-dioxide 56e)

The compound was obtained from 53e by following the experimental conditions described for 54e: ¹H NMR (400 MHz, DMSO-d₆): δ=3.73 (s, 3H), 4.40 (2H, d, J=5.6 Hz), 6.92 (2H, d, J=8.6 Hz), 7.26 (m, 3H), 7.62 (2H, dd, J=8.7, 2.4 Hz), 7.62 (1H, d, J=2.4 Hz), 10.75 ppm (br, 1H). ¹³C NMR (100 MHz, DMSO-d₆): δ=43.87, 55.55, 114.29, 119.40, 122.61, 124.26, 127.59, 129.17, 130.70, 130.90, 132.87, 135.19, 151.51, 158.94 ppm. HRMS (ESI): m/z calcd for C₁₅H₁₄ClN₃O₃S [M+Na]⁺: 374.03421, found: 374.0338.

### 3-(Benzylamino)-7-chloro-4H-benzo[e][1,2,4]thiadiazine 1,1-dioxide (57e)

The compound was obtained from 53e by following the experimental conditions described for 54e: ¹H NMR (400 MHz, DMSO-d₆): δ=4.47 (s, 2H), 7.25 (m, 2H), 7.34 (m, 4H), 7.69 (1H, d, J=8.7 Hz), 7.66 (s, 2H), 10.69 ppm (br, 1H). ¹³C NMR (100 MHz, DMSO-d₆): δ=44.34, 119.51, 122.59, 124.25, 127.51, 127.57, 127.67, 127.77, 128.86, 132.84, 135.39, 135.42, 138.92, 151.47, 151.75 ppm. HRMS (ESI): m/z calcd for C₁₄H₁₂ClN₃O₂S [M+Na]⁺: 344.0236, found: 344.0308.

### 7-Chloro-3-((3-methylbenzyl)amino)-4H-benzo[e][1,2,4]thiadiazine1,1-dioxide (58e)

The compound was obtained from 53e by following the experimental conditions described for 54e: ¹H NMR (400 MHz, DMSO-d₆): δ=2.30 (s, 3H), 4.45 (2H, d, J=5.6 Hz), 7.35 (m, 3H), 7.24 (2H, t, J=7.8 Hz), 7.61 (1H, dd, J=8.7, 2.3 Hz), 7.68 (1H, d, J=2.4 Hz), 10.78 ppm (br, 1H). ¹³C NMR (100 MHz, DMSO-d₆): δ=21.49, 44.32, 119.41, 122.61, 124.25, 124.76, 127.63, 128.23, 128.26, 128.80, 132.89, 135.15, 137.98, 138.673, 151.58 ppm. HRMS (ESI): m/z calcd for C₁₅H₁₄ClN₃O₂S [M+Na]⁺: 358.0392, found: 358.0387.

### 7-Chloro-3-(cyclopentylamino)-4H-benzo[e][1,2,4]thiadiazine 1,1-dioxide (59e)

The compound was obtained from 53e by following the experimental conditions described for 54e: ¹H NMR (400 MHz, DMSO-d₆): δ=1.44-1.66 (m, 6H), 1.90 (m, 2H), 4.06 (m, 1H), 7.24 (2H, d, J=7.8 Hz), 7.58 (2H, dd, J=8.7, 2.3 Hz), 7.65 (2H, d, J=2.3 Hz), 10.39 ppm (s, 1H). ¹³C NMR (100 MHz, DMSO-d₆): δ=23.64, 32.69, 52.85, 119.38, 122.55, 124.33, 127.55, 132.79, 135.04, 151.11 ppm. HRMS (ESI): m/z calcd for C₁₂H₁₄ClN₃O₂S [M+Na]⁺: 322.0392, found: 322.0351.

### 7-Chloro-3-((3-phenylpropyl)amino)-4H-benzo[e][1,2,4]thiadiazine 1,1-dioxide (60e)

The compound was obtained from 53e by following the experimental conditions described for 54e: ¹H NMR (400 MHz, DMSO-d₆): δ=1.83 (m, 2H), 2.64 (2H, t, J=8 Hz), 3.26 (q, 2H), 7.17-7.31 (m, 7H), 7.55 (1H, dd, J=8.7, 2.4 Hz), 7.66 (1H, d, J=2.4), 10.71 ppm (s, 1H). ¹³C NMR (100 MHz, DMSO-d₆): δ=30.96, 32.79, 119.33, 122.58, 124.27, 124.30, 126.29, 127.53, 128.74, 128.80, 128.91, 132.82, 135.16, 141.86, 151.55 ppm. HRMS (ESI): m/z calcd for C₁₆H₁₆ClN₃O₂S [M+Na]⁺: 372.0549, found: 372.0546.

### 7-Chloro-3-((4-methylbenzyl)amino)-4H-benzo[e][1,2,4]thiadiazine 1,1-dioxide (61e)

The compound was obtained from 53e by following the experimental conditions described for 54e: ¹H NMR (400 MHz, DMSO-d₆): δ=2.29 (s, 3H), 4.43 (2H, d, J=5.7 Hz), 7.17 (2H, d, J=8.2 Hz), 7.22 (2H, d, J=8.2 Hz), 7.26 (s, 1H), 7.30 (s, 1H), 7.66 (1H, dd, J=8.7, 2.4 Hz), 7.68 (2H, d, J=2.4), 10.82 ppm (br, 1H). ¹³C NMR (100 MHz, DMSO-d₆): δ=21.14, 44.12, 119.38, 122.61, 124.25, 127.63, 127.69, 129.41, 132.89, 135.13, 135.76, 136.71, 151.55 ppm.

HRMS (ESI): m/z calcd for C₁₅H₁₄ClN₃O₂S [M+Na]⁺: 358.0392, found: 358.0389.

### 7-Chloro-3-((3-methoxybenzyl)amino)-4H-benzo[e][1,2,4]thiadiazine 1,1-dioxide (62e)

The compound was obtained from 53e by following the experimental conditions described for 54e: ¹H NMR (400 MHz, DMSO-d₆): δ=3.74 (s, 3H), 4.45 (2H, d, J=5.7 Hz), 6.86 (1H, d, J=7.2 Hz), 6.92 (2H, d, J=8.7 HZ), 7.27 (1H, t, J=8.4 Hz), 7.61 (1H, d, J=8.7 Hz), 7.67 (s, 2H), 10.84 ppm (br, 1H). ¹³C NMR (100 MHz, DMSO-d₆): δ=44.29, 55.48, 113.02, 113.37, 119.41, 119.80, 122.62, 124.24, 127.67, 129.97, 132.91, 135.31, 140.45, 151.58, 159.82 ppm. HRMS (ESI): m/z calcd for C₁₅H₁₄ClN₃O₃S [M+Na]⁺: 374.0342, found: 374.0338.

### 7-Chloro-3-((2,4-difluorobenzyl)amino)-4H-benzo[e][1,2,4]thiadiazine 1,1-dioxide (63e)

The compound was obtained from 53e by following the experimental conditions described for 54e: ¹H NMR (400 MHz, DMSO-d₆): δ=4.48 (2H, d, J=5.7 Hz), 7.09 (1H, t, J=8.7 Hz), 7.24 (m, 2H), 7.44 (q, 1H), 7.67 (m, 3H), 10.84 ppm (br, 1H). ¹³C NMR (100 MHz, DMSO-de): δ=38.10, 104.26, 104.57, 111.74, 111.95, 119.43, 122.07, 122.65, 124.14, 127.76, 131.18, 132.94, 135.06, 151.52 ppm. HRMS (ESI): m/z calcd for C₁₅H₁₀ClF₂N₃O₂S [M+Na]⁺: 380.0048, found: 380.0041.

### 7-Chloro-3-((4-methylbenzyl)amino)-4H-benzo[e][1,2,4]thiadiazine1,1-dioxide (64e)

The compound was obtained from 53e by following the experimental conditions described for 54e: ¹H NMR (400 MHz, DMSO-d₆): δ=2.26 (s, 3H), 4.48 (2H, d, J=4.8 Hz), 7.20 (m, 3H), 7.26 (m, 2H), 7.40 (2H, dd, J=8.8, 2.4 Hz), 7.67 (s, 1H), 10.71 ppm (br, 1H). ¹³C NMR (100 MHz, DMSO-d₆): δ=19.09, 42.45, 119.44, 122.62, 124.22, 126.35, 127.60, 127.65, 130.76, 132.88, 135.18, 136.03, 136.48, 151.60 ppm. HRMS (ESI): m/z calcd for C₁₅H₁₄ClN₃O₂S [M+Na]⁺: 358.0392, found: 358.0399.

### 7-Chloro-3-((4-phenylbutyl)amino)-4H-benzo[e][1,2,4]thiadiazine 1,1-dioxide (65e)

The compound was obtained from 53e by following the experimental conditions described for 54e: ¹H NMR (400 MHz, DMSO-d₆): δ=1.53 (m, 4H), 2.61 (2H, t, J=7.7 Hz), 3.26 (2H, t, J=6.8 Hz), 7.22 (m, 6H), 7.61 (1H, d, J=8.8 Hz), 7.66 (s, 1H), 10.67 ppm (br, 1H). HRMS (ESI): m/z calcd for C₁₇H₁₈ClN₃O₂S [M+Na]⁺: 386.8486, found: 386.8473.

### 7-Chloro-3-((pyridin-4-ylmethyl)amino)-4H-benzo[e][1,2,4]thiadiazine 1,1-dioxide (66e)

The compound was obtained from 53e by following the experimental conditions described for 54e: ¹H NMR (400 MHz, DMSO-d₆): δ=4.50 (2H, d, J=5.7 Hz), 7.29 (1H, d, J=8.8 Hz), 7.32 (1H, d, J=8.8 Hz), 7.62 (1H, dd, J=8.7, 2.0 Hz), 7.68 (1H, d, J=2.0 Hz), 7.77 (br, 1H), 8.53 (2H, d, J=4.6 Hz), 11.09 ppm (br, 1H). ¹³C NMR (100 MHz, DMSO-d₆): δ=40.40, 119.44, 122.40, 122.64, 124.15, 127.78, 132.97, 135.10, 148.12, 150.04, 151.71 ppm. HRMS (ESI): m/z calcd for C₁₃H₁₁ClN₄O₂S [M+Na]⁺: 345.7571, found: 345.7448.

### 7-Chloro-3-((4-(trifluoromethoxy)benzyl)amino)-4H-benzo[e][1,2,4]thiadiazine 1,1-dioxide (67e)

The compound was obtained from 53e by following the experimental conditions described for 54e: ¹H NMR (400 MHz, DMSO-d₆): δ=4.50 (2H, d, J=5.8 Hz), 7.32 (m, 4H), 7.48 (2H, d, J=8.7 Hz), 7.71 (1H, d, J=8.7 Hz), 7.84 (br, 1H), 10.89 ppm (s, 1H). ¹³C NMR (100 MHz, DMSO-d₆): δ=16.54, 120.49, 126.17, 127.28, 128.99, 137.07, 137.80, 142.87, 147.28, 154.95, 166.44, 168.31 ppm. HRMS (ESI): m/z calcd for C₁₅H₁₁ClF₃N₃O₃S [M+Na]⁺: 428.0059, found: 428.0031.

### 7-Chloro-3-((1-phenylethyl)amino)-4H-benzo[e][1,2,4]thiadiazine 1,1-dioxide (68e)

The compound was obtained from 53e by following the experimental conditions described for 54e: ¹H NMR (400 MHz, DMSO-d₆): δ=1.49 (3H, d, J=6.9 Hz), 5.02 (m, 1H), 7.24 (m, 2H), 7.38 (m, 4H), 7.60 (1H, dd, J=8.7, 2.4 Hz), 7.66 (1H, d, J=2.4 Hz), 7.74 (br, 1H), 10.62 ppm (s, 1H). ¹³C NMR (100 MHz, DMSO-d₆): δ=22.88, 50.49, 119.42, 122.61, 126.43, 127.58, 127.69, 128.93, 132.89, 134.96, 143.77, 150.80 ppm. HRMS (ESI): m/z calcd for C₁₅H₁₄ClN₃O₂S [M+Na]⁺: 358.0392, found: 358.0385.

### 6-Chloro-3-((4-methoxybenzyl)amino)-4H-benzo[e][1,2,4]thiadiazine 1,1-dioxide (20b)

The compound was obtained from 13b by following the experimental conditions described for 54e: ¹H NMR (400 MHz, DMSO-d₆): δ=3.73 (s, 3H), 4.40 (s, 2H), 6.92 (2H, d, J=8.5 Hz), 7.28 (m, 4H), 7.69 (1H, d, J=8.7 Hz), 7.74 ppm (br, 1H). ¹³C NMR (100 MHz, DMSO-de): δ=43.87, 55.51, 114.25, 116.63, 121.87, 124.23, 125.41, 129.19, 130.65, 130.90, 137.01, 137.60, 151.40, 158.93 ppm. HRMS (ESI): m/z calcd for C₁₅H₁₄ClN₃O₃S [M+Na]⁺: 374.0342, found: 374.0332.

### 3-(Benzylamino)-6-chloro-4H-benzo[e][1,2,4]thiadiazine 1,1-dioxide (21b)

The compound was obtained from 13b by following the experimental conditions described 54e ¹H NMR (400 MHz, DMSO-d₆): δ=4.48 (s, 2H), 7.29 (m, 3H), 7.34 (m, 4H) 7.69 (1H, d, J=8.8 Hz), 7.82 (br, 1H), 10.46 ppm (br, 1H). ¹³C NMR (100 MHz, DMSO-d₆): δ=44.34, 116.69, 121.88, 124.23, 125.42, 127.58, 127.68, 128.87, 136.99, 137.69, 138.86, 151.57 ppm. HRMS (ESI): m/z calcd for C₁₄H₁₂ClN₃O₂S [M+Na]⁺: 344.0236, found: 344.0235.

### 6-Chloro-3-((3-phenylpropyl)amino)-4H-benzo[e][1,2,4]thiadiazine 1,1-dioxide (23b)

The compound was obtained from 13b by following the experimental conditions described for 54e: ¹H NMR (400 MHz, DMSO-d₆): δ=1.83 (m, 2H), 2.63 (2H, t, J=7.8 Hz), 3.23 (q, 2H), 7.17-7.37 (m, 8H), 7.67 (1H, d, J=8.5 Hz), 10.66 ppm (s, 1H). ¹³C NMR (100 MHz, DMSO-d₆): δ=30.93, 31.16, 32.79, 116.59, 121.92, 124.18, 125.40, 126.29, 128.75, 128.80, 136.90, 137.60, 141.86, 151.44 ppm. HRMS (ESI): m/z calcd for C₁₆H₁₆ClN₃O₂S [M+Na]⁺: 372.0549, found: 372.0544.

### 6-Chloro-3-((4-methylbenzyl)amino)-4H-benzo[e][1,2,4]thiadiazine 1,1-dioxide (27b)

The compound was obtained from 13b by following the experimental conditions described for 54e: ¹H NMR (400 MHz, DMSO-d₆): δ=2.28 (s, 3H), 4.41 (2H, d, J=4.2 Hz), 7.17 (2H, d, J=7.9 Hz), 7.22 (2H, d, J=7.9 Hz), 7.28 (s, 1H), 7.30 (s, 1H), 7.68 (1H, d, J=8.3 Hz), 7.76 (br, 1H), 10.64 ppm (br, 1H). ¹³C NMR (100 MHz, DMSO-d₆): δ=21.14, 44.10, 116.66, 121.89, 124.23, 125.42, 127.70, 129.40, 135.76, 136.70, 136.96, 137.65, 151.49 ppm. HRMS (ESI): m/z calcd for C₁₅H₁₄ClN₃O₂S [M+Na]⁺: 358.0392, found: 358.0388.

### 6-Chloro-3-((3-methoxybenzyl)amino)-4H-benzo[e][1,2,4]thiadiazine 1,1-dioxide (28b)

The compound was obtained from 13b by following the experimental conditions described for 54e: ¹H NMR (400 MHz, DMSO-d₆): δ=3.74 (s, 3H), 4.46 (2H, d, J=5.3 Hz), 6.85 (1H, dd, J=8, 2 Hz), 6.94 (m, 2H), 7.27 (1H, d, J=7.8 Hz), 7.29 (2H, d, J=7 Hz), 7.70 (1H, d, J=8.8 Hz), 7.79 (br, 1H), 10.79 ppm (s, 1H). ¹³C NMR (100 MHz, DMSO-d₆): δ=44.29, 55.45, 112.99, 113.38, 116.66, 119.81, 121.78, 124.30, 125.43, 129.95, 137.03, 137.54, 140.42, 151.47, 159.81 ppm. HRMS (ESI): m/z calcd for C₁₅H₁₄ClN₃O₃S [M+Na]⁺: 374.0342, found: 374.0335.

### 6-Chloro-3-((2,4-difluorobenzyl)amino)-4H-benzo[e][1,2,4]thiadiazine 1,1-dioxide (29b)

The compound was obtained from 13b by following the experimental conditions described for 54e: ¹H NMR (400 MHz, DMSO-d₆): δ=4.47 (2H, d, J=5.6 Hz), 7.10 (1H, t, J=8.5 Hz), 7.24-7.32 (m, 3H), 7.45 (q, 1H), 7.69 (1H, d, J=8.5 Hz), 7.79 (br, 1H), 10.84 ppm (s, 1H). ¹³C NMR (100 MHz, DMSO-d₆): δ=38.08, 104.26, 111.77, 116.67, 121.78, 124.40, 125.47, 131.23, 137.04, 137.44, 151.38, 159.34, 160.77, 161.68, 163.33 ppm. HRMS (ESI): m/z calcd for C₁₅H₁₀ClF₂N₃O₂S [M+Na]⁺: 380.0048, found: 380.0051.

### 7-Bromo-3-((2,4-difluorobenzyl)amino)-4H-benzo[e][1,2,4]thiadiazine 1,1-dioxide (29c)

The compound was obtained from 13c by following the experimental conditions described for 54e: ¹H NMR (400 MHz, DMSO-d₆): δ=4.48 (2H, d, J=5.5 Hz), 7.10 (1H, t, J=8.5 Hz), 7.20 (1H, d, J=8.7 Hz), 7.27 (m, 1H), 7.45 (q, 1H), 7.70 (br, 1H), 7.73 (1H, dd, J=8.7, 2.2 Hz), 7.78 (1H, d, J=2.2 Hz), 10.93 ppm (s, 1H). ¹³C NMR (100 MHz, DMSO-d₆): δ=38.09, 104.27, 111.99, 115.20, 119.66, 122.06, 124.45, 125.42, 131.20, 135.39, 135.69, 151.43, 159.20, 160.76, 161.79, 163.32 ppm. HRMS (ESI): m/z calcd for C₁₅H₁₀BrF₂N₃O₂S [M+Na]⁺: 423.9542, found: 423.9538.

### 6-Chloro-3-((4-(trifluoromethoxy)benzyl)amino)-4H-benzo[e][1,2,4]thiadiazine 1,1-dioxide (30b)

The compound was obtained from 13b by following the experimental conditions described for 54e: ¹H NMR (400 MHz, DMSO-d₆): δ=4.51 (2H, d, J=5.7 Hz), 7.30 (2H, d, J=7.8 Hz), 7.37 (2H, d, J=8.6 Hz), 7.45 (2H, d, J=8.6 Hz), 7.69 (1H, d, J=8.6 Hz), 7.85 (br, 1H), 10.90 ppm (s, 1H). ¹³C NMR (100 MHz, DMSO-d₆): δ=43.65, 116.66, 119.26, 121.51, 121.81, 121.85, 124.35, 125.45, 129.54, 137.03, 137.52, 138.49, 147.84, 151.47 ppm. HRMS (ESI): m/z calcd for C₁₅H₁₁ClF₃N₃O₃S [M+Na]⁺: 428.0059, found: 428.0062.

### 6-Chloro-3-((3-methoxyphenethyl)amino)-4H-benzo[e][1,2,4]thiadiazine 1,1-dioxide (40b)

The compound was obtained from 13b by following the experimental conditions described for 54e: ¹H NMR (400 MHz, DMSO-d₆): δ=2.83 (2H, t, J=7.1 Hz), 3.48 (q, 2H), 3.74 (s, 3H), 6.80 (1H, d, J=8.2 Hz), 6.87 (m, 2H), 7.23 (m, 3H), 7.29 (1H, dd, J=8.4, 2 Hz), 7.68 (1H, d, J=8.4 Hz), 10.67 ppm (br, 1H). ¹³C NMR (100 MHz, DMSO-d₆): δ=34.98, 42.29, 55.36, 112.19, 114.78, 116.53, 121.37, 121.86, 124.23, 125.42, 129.91, 136.94, 137.51, 140.86, 151.38, 159.79 ppm. HRMS (ESI): m/z calcd for C₁₆H₁₆ClN₃O₃S [M+Na]⁺: 388.0498, found: 388.0542.

### 6-Chloro-3-((3-methylbenzyl)amino)-4H-benzo[e][1,2,4]thiadiazine 1,1-dioxide (42b)

The compound was obtained from 13b by following the experimental conditions described for 54e: ¹H NMR (400 MHz, DMSO-d₆): δ=2.29 (s, 3H), 4.44 (2H, d, J=5 Hz), 7.05-7.15 (m, 3H), 7.24 (1H, t, J=7.5 Hz), 7.29 (2H, J=9.0 Hz), 7.70 (1H, d, J=7.5 Hz), 7.70 (1H, d, J=8.2 Hz), 7.75 (br, 1H), 10.60 ppm (br, 1H). ¹³C NMR (100 MHz, DMSO-d₆): δ=21.48, 44.32, 116.68, 121.91, 124.26, 123.78, 125.43, 128.23, 128.28, 128.80, 136.97, 137.62, 137.97, 138.72, 151.49 ppm. HRMS (ESI): m/z calcd for C₁₅H₁₄ClN₃O₂S [M+Na]⁺: 358.0392, found: 358.0339.

### 6-Chloro-3-((2-methylbenzyl)amino)-4H-benzo[e][1,2,4]thiadiazine 1,1-dioxide (41b)

The compound was obtained from 13b by following the experimental conditions described for 54e: ¹H NMR (400 MHz, DMSO-d₆): δ=2.32 (s, 3H), 4.41 (s, 2H), 7.19 (s, 3H), 7.28 (3H, t, J=9 Hz), 7.64 (br, 1H), 7.67 (1H, d, J=8.2 Hz), 10.33 ppm (br, 1H). ¹³C NMR (100 MHz, DMSO-d₆): δ=19.09, 42.53, 116.73, 121.90, 124.24, 125.43, 126.34, 127.65, 127.72, 130.52, 136.04, 136.48, 136.97, 151.56 ppm. HRMS (ESI): m/z calcd for C₁₅H₁₄ClN₃O₂S [M+Na]⁺: 358.0392, found: 358.0287.

### 6-Chloro-3-((2-(trifluoromethoxy)benzyl)amino)-4H-benzo[e][1,2,4]thiadiazine 1,1-dioxide (43b)

The compound was obtained from 13b by following the experimental conditions described for 54e: ¹H NMR (400 MHz, DMSO-d₆): δ=4.55 (2H, d, J=5.8 Hz), 7.30 (2H, d, J=7.8 Hz), 7.32 (2H, d, J=8.6 Hz), 7.47 (2H, d, J=8.6 Hz), 7.75 (br, 1H), 7.84 (1H, d, J=8.6 Hz), 10.93 ppm (s, 1H). ¹³C NMR (100 MHz, DMSO-d₆): δ=113.89, 116.66, 120.82, 121.07, 124.43, 125.50, 126.16, 126.52, 128.01, 129.63, 131.20, 137.25, 157.85, 151.52, 161.90 ppm. HRMS (ESI): m/z calcd for C₁₅H₁₁ClF₃N₃O₃S [M+Na]⁺: 428.0059, found: 428.0021.

### Example 2: Mitochondrial Respiratory Complex II Inhibition Assay

The assay was performed as follows. Mitochondria were isolated either from fresh rat hearts or by differential centrifugation in sucrose-based buffer as previously described.²⁴ Complex II enzymatic activity was determined spectrophotometrically as the rate of succinate-driven, co-enzyme Q2-linked reduction of dichlorophenolindophenol (DCPIP).⁴⁹ Mitochondria or sub-mitochondrial particles were incubated in phosphate buffer (pH 7.4) containing 40 μM DCPIP, 1 mM KCN, 10 μM rotenone, and 50 μM co-enzyme Q2. The rate of reduction of DCPIP to DCPIPH2 was followed at 600 nM (ε=21,000 M-1). Varying amounts of succinate and inhibitors were used to determine an IC₅₀ value. At the end of each run thenoyltrifluoroacetone (1 mM) was added and the residual TTFA-insensitive rate subtracted.

Mitochondrial respiratory complex II activity was measured spectrophotometrically using isolated rat heart mitochondria, with suitable modifications to ensure rapid isolation as previously described.²⁴ The natural product and potent CII inhibitor Atpenin A5 (6), IC₅₀=3.3 nM,^{24, 29} was employed as positive control with DMSO as negative control. The parent compound diazoxide (9), was found to be inactive with no inhibition activity at 100 μM and a calculated IC₅₀>1000 μM (Table 3) compared with the value of 32 μM reported in the literature.³¹ The positive control compound 6 induced 93% inhibition at 0.1 μM, validating the assay protocol. To unequivocally associate this activity to the parent compound employed both synthesized and commercially acquired samples of 9. While surprising, this inactivity was confirmed in the prostate and breast cancer cell lines wherein 9 had no effect on cell viability (see below). Indeed, when the literature is more closely examined most studies employ a concentration of 9 much greater than the reported 32 μM with some experiments performed up to 750 μM.^{31, 33, 34}

Undeterred by the apparent lack of CII inhibition activity of parent compound (IC₅₀=1236 μM) derivatives of 9 were screened for CII inhibition. All of the synthesized derivatives were initially screened for their CII inhibition potential at 100 μM. Halogen substitution on the benzothiadiazine ring provided increased CII inhibition activity over saturated counterparts. Among 7-fluorobenzothiadiazine substituted derivatives (Scheme 3 Å), 4-chlorobenzylamine (16a) provided 30% inhibition, when the chain length was extended, the 4-chlorophenethyamine derivative (37a) induced 34% inhibition. Replacement of the electron-withdrawing chlorine on the side chain benzylamine with an electron-donating methyl substituent to afford 4-methylbenzylamine derivative 27a, inhibition was decreased to 23%. Replacement with a highly electron withdrawing trifluoromethyl substituent (25a) afforded an inactive compound, just 7% inhibition at 100 μM, while the 2-methoxybenzylamine (26a) induced 37% inhibition. The most active derivative of the 7-fluoro series was allylamine 32a, inducing 38% inhibition, equipotent with 26a, indicating that a side chain containing an aromatic ring is not required for activity.

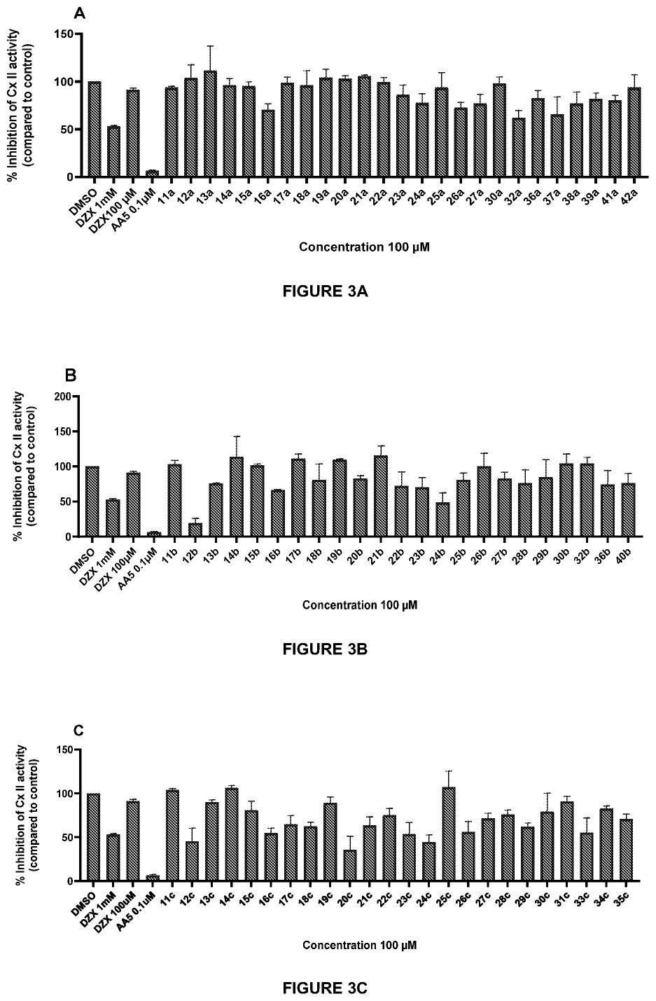

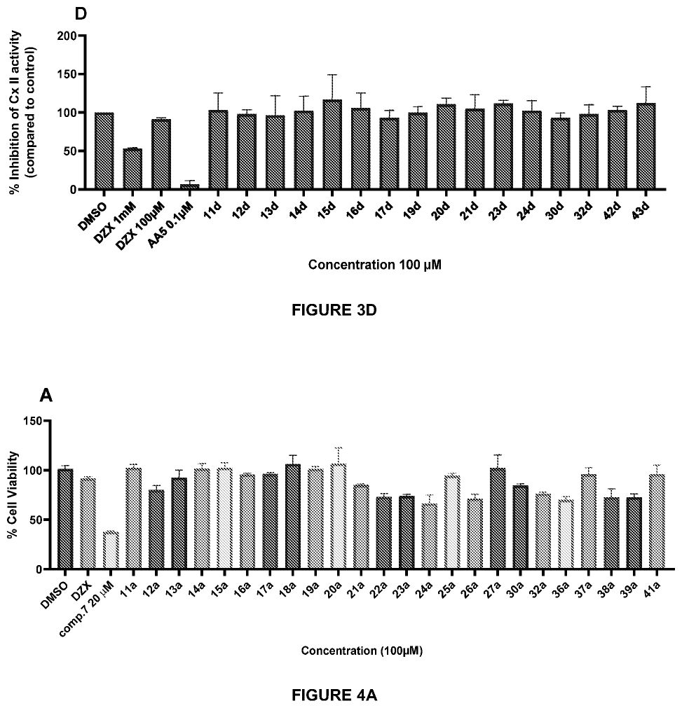

When the 7-fluoro substituent on the benzothiadiazine ring was switched to a 6-chloro substituent (FIG. 3B) the unfunctionalized thiourea derivative (12b) exhibited startlingly potent inhibition activity (81% inhibition at 100 μM). However, the chromophoric nature of the compound was determined to interfere with the assay readout, leading to what seems to be a false positive. The compound shares structure similarity to a number of known promiscuous compounds known as pan-assay interfering compounds (PAINS).⁴⁵ The 4-chlorobenzylamine derivative (16b) induced 34% inhibition, equipotent with its 7-fluoro benzothiadiazine substituted counterpart 16a. Cyclopentamine derivative 22b induced 27% inhibition, more active than the respective 7-fluoro benzothiadiazine substituted compound 22a (inactive). The H-Indole-3-ethylamine side chain substituted compound (36b) induced 25% inhibition. The 7-fluoro benzothiadiazine substituted derivatives with the same side chain; 3-phenylpropylamine (23a) induces just 14% inhibition while H-Indole-3-ethylamine (36a) is equipotent. The most active derivative from the 6-chloro series possessed a 1-phenylethylamine side chain (24b) inducing 51% CII inhibition at 100 μM, possibly indicating a role for the phenyl ring in pi-pi stacking at this position of the molecule. Overall, the 6-chloro substitution pattern on the benzothiadiazine ring provided no appreciable increase in inhibition activity compared to 7-fluoro substitution.

When the 7-fluoro substituent on the benzothiadiazine ring was interchanged with 7-bromo substitution the inhibitory activity of the derivatives notably increased (FIG. 3C). The unfunctionalized thiourea derivative 12c was active, inducing 55% inhibition of CII at 100 μM, compared with 81% with the 6-chloro substituted benzothiadiazine. However, the chromophore of the compound was again found to interfere with the assay results; the structure again acting in the role of PAINS. The 4-chlorobenzylamine derivative (16c) induced 45% inhibition of CII at 100 μM with the 7-bromo substituted benzothiadiazine ring, conferring increased activity over its 7-fluoro (16b) and 6-chloro (16a) counterparts and in contrast to the inactive unsubstituted derivative 16d. Equipotent inhibition to 16c was noted with 3-phenylpropylamine (23c), which induced 46% inhibition respectively. Again the 7-bromo substituted benzothiadiazine ring was more active than the 6-chloro substituted 3-phenylpropylamine (23a) and the unsubstituted derivative 23d, which induced 14% and 0% inhibition respectively. The 1-phenylethylamine derivative (24c) induced 55% inhibition of CII at 100 μM equipotent with its 6-chloro counterpart (24b). The most active compound identified in this study, 4-methoxybenzylamine (20c), outside of the chromophoric false positives, induced 64% inhibition at 100 μM. The compounds possessing an unsubstituted benzothiadiazine ring (FIG. 3D) exhibited no inhibition of CII at 100 μM.

A preliminary structure-activity relationship can be derived for CII inhibition activity of this scaffold. Halogen substitution at the 6- or 7-position of the benzothiadiazine ring affords for inhibition activity which is completely absent from the respective saturated derivatives. Of all halogen substituents evaluated herein, 7-bromo represents the most active inhibitors. The side chain derivatives require either aromatic or possibly allyl (in the case of a 7-F substituted benzothiadiazine ring, but interestingly not when combined with 6-Cl substitution) moieties to confer CII inhibition activity. However, no clear substituent pattern can be derived beyond 4-CF₃ is deleterious to activity (25a and 25c confer 0% inhibition while 25b induces only 19% inhibition). Alkyl side chains yield inactive compounds. However, a cyclopentane ring does provide some activity (approximately 25% inhibition).

Five of the most active CII inhibitors at 100 μM (12b, 81% inhibition; 12c, 55%; 20c, 64%; 24b, 51% and 24c, 55%), the parent compound diazoxide (9, 9% inhibition at 100 μM) and positive control Atpenin A5 (6, 93% inhibition at 0.1 μM) were selected for IC₅₀ determination (Table 3). The parent compound 9 possessed an IC₅₀=1236 μM, greatly reduced activity over the 32 μM IC₅₀ reported in the literature.³¹ Positive control compound 6 possessed an IC₅₀=3.3 nM, in accordance with literature values.⁴⁵ The two unfunctionalized sulfonylureas 12b and 12c displayed the most potent IC₅₀ values of 11.88 and 36.98 μM respectively as expected from the initial compound screen at 100 μM. However, it should be again noted these compounds are expected to be false positive PAINS. The most active compound identified in the initial screen 20c, possessed an IC₅₀=79.68 μM. The 6-chloro substituted 1-phenylethylamine side chain derivative 24b possessed an IC₅₀=89 μM and its 7-fluoro counterpart (24c) an IC₅₀=79.82 μM. The obtained IC₅₀ values directly correlate with the activity pattern obtained in the initial screen conducted at 100 μM. Several novel diazoxide derivatives have been identified with significantly increased activity to inhibit CII, with the most active compounds conferring >15-fold increased potency, albeit with only moderate activity compared to known inhibitors (FIG. 2).

TABLE 3
Mitochondrial respiratory complex II IC₅₀ values of selected
diazoxide derivatives.

| Compound | Mw | ClogP^{a} | PSA^{b} | CII IC₅₀ (μM)^{c} |
|---|---|---|---|---|
| Diazoxide (9) | 230.67 | 1.0 | 58.53 | 1236 ± 2.5 |
| Atpenin A5 (6) | 366.24 | 2.64 | 88.88 | 0.0033 ± 2 |
| 12b | 248.7 | 1.33 | 58.2 | 11.88* ± 3.3 |
| 12c | 293.15 | 1.48 | 58.2 | 36.98* ± 2.4 |
| 20c | 396.26 | 2.30 | 79.79 | 79.68 ± 4 |
| 24b | 335.81 | 2.54 | 70.56 | 89.01 ± 10.4 |
| 24c | 380.26 | 2.69 | 70.56 | 79.82 ± 4.1 |

^{a}Calculated by ChemDraw Professional 16.0.
^{b}Polar surface area (pH 7.4), calculated by ChemDraw Professional 16.0.
^{c}Values are the mean ±SD of n = 4 experiments.
*Probable PAINS

### Example 3: Cytotoxicity Assays

The assay was performed as follows. To determine the cell growth inhibition ability of the synthesized compounds the (3-(4,5-dimethylthiazol-2-yl)-5-(3-carboxymethoxyphenyl)-2-(4-sulfophenyl)-2H-tetrazolium) (MTS) assay used according to the manufacturer's recommended protocol. Stock solutions of the synthesized compounds were prepared in DMSO. Cells were seeded at a density of 1×10⁵ cells in 96-well plates. After 24 hours, cells were treated at the indicated concentrations of test compounds, limiting the final DMSO concentration to less than 1%. After incubation at 37° C. in an environment of 5% CO₂ for 48-72 hours, 10 μL of MTS reagent (CellTiter 96© AQueous One Solution Reagent) was added to each well and incubated at the above mentioned conditions for 2-4 hr. Absorbance was recorded at 490 nm on a BioTek Synergy Mx multimode plate reader and the viability of cells were plotted as percentage of controls.

Cell lines (Primary Human Umbilical Vein Endothelial Cells (HUVEC), human embryonic kidney cells (HEK293), 22Rv1 prostate cancer and MDA-MB 468 triple-negative breast cancer cells) were purchased from ATCC. The HUVEC cells were routinely cultured in M199 medium (Corning, Cat #MT10060CV), supplemented with 15% Fetal Bovine Serum (FBS, GIBCO™, Cat #10438026), 150 μg/mL Endothelial Cell Growth Supplement (ECGS), 5 U/mL heparin sodium and 1× Antibiotic-Antimycotic solution (GIBCO™, Cat #15240-062). MDA-MB-468 cells were cultured in Dulbecco's Modified Eagle Medium (DMEM, Fisher Scientific, Cat #50-188-267FP) and HEK293 cells in Eagle's Minimum Essential Medium (ATCC®, Cat #30-2003™), supplemented with FBS (ATCC®, Cat #30-2020) to a final concentration of 10% and Penicillin-Streptomycin Solution (Corning™, Cat #MT30001CI) according to the supplier's recommended protocol. The 22Rv1 prostate cancer and MDA-MB 468 cells were cultured in RPMI-1640 Medium (ATCC® 30-2001™) for 22Rv1 cells and in Dulbecco's Modified Eagle Medium (DMEM) (ThermoFisher Scientific) for MDA-MB 468 cells with fetal bovine serum (ATCC 30-2020) to a final concentration of 10% and Corning™ Penicillin-Streptomycin Solution (Catalog No. MT30001CI) according to the supplier's recommended protocol.

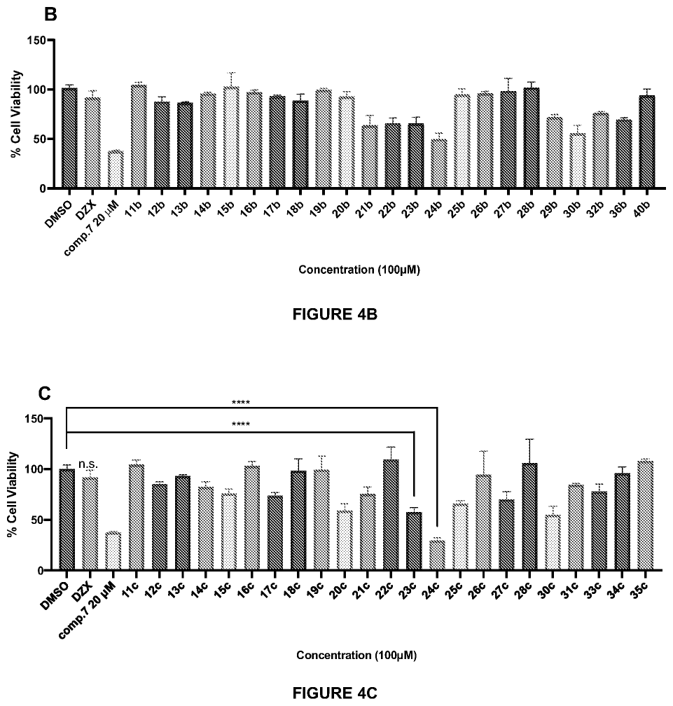

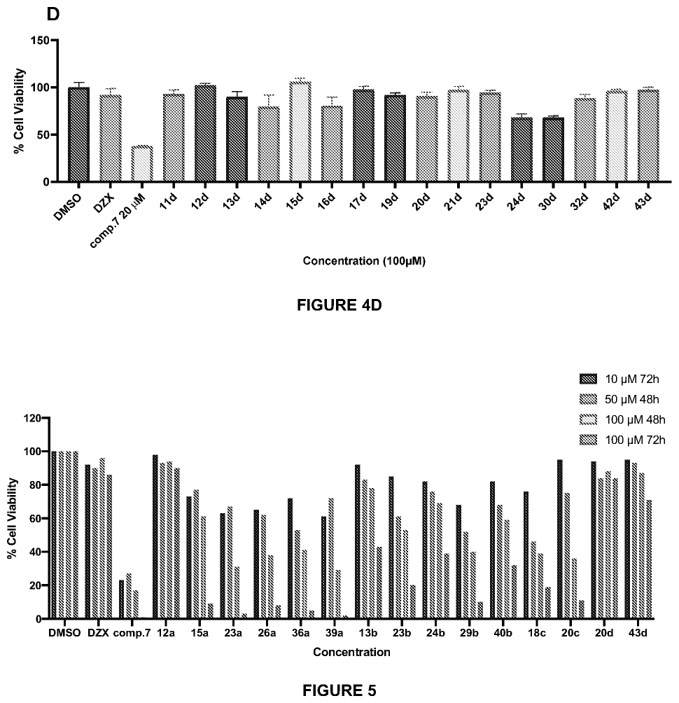

The cytotoxicity of the diazoxide derivatives at 100 μM concentration was determined in 22Rv1 prostate cancer cells after 48 hrs treatment employing the 3-(4,5-dimethylthiazol-2-yl)-5-(3-carboxymethoxyphenyl)-2-(4-sulfophenyl)-2H-tetrazolium (MTS) assay as previously reported.⁴⁶ Atpenin A5 derivative 7, (FIG. 2) which possesses a CII IC₅₀=64 nM and a ‘drug-like’ ligand-lipophilicity efficiency of 5.62 was employed as a positive control. This compound has been previously reported to reduce cell viability of 22Rv1 cells.³⁰ In this assay, 7 provided a significant inhibitory effect at 20 μM concentration, reducing cell viability by 60%. The parent compound 9, despite lacking any CII inhibition activity at 100 μM, reduced 22Rv1 prostate cancer cell survival by 12% (FIG. 4).

The 7-fluorobenzothiadiazine substituted derivatives generally provided the least effect on 22Rv1 prostate cancer cell viability of all of the halogen substituted derivatives. The most potent CII inhibitor from this series, allylamine (32a) displaying 38% CII inhibition, afforded 24% reduction of cell viability (FIG. 4A). However, this derivative was not the most cytotoxic in the 22Rv1 cells; 1-Phenylethylamine (24a) which possess 22% CII inhibition affords 34% reduction in cell viability while the 3-Indoleethylamine derivative (36a) which possesses 17% CII inhibition activity induces 30% reduction of cell viability in 22Rv1 cells. The 4-chlorobenzylamine 16a (30% CII inhibition) and 4-chlorophenethylamine homologue 37a (34% CII inhibition) both proved inactive in 22Rv1 cells.

From the 6-chlorobenzothiadiazine substituted derivatives the most active compound, 1-phenylethylamine (24b) (51% CII inhibition at 100 μM, IC₅₀=89.0±10.4 μM) afforded a 50% reduction in 22Rv1 cell viability (FIG. 4B). However, the inactive CII inhibitor benzylamine (21b) and the moderately activity cyclopentylamine (22b), which possesses 27% CII inhibition and 3-phenylpropylamine (23b) which possess 30% CII inhibition activity at 100 μM were all equipotent to reduce cell viability of 22Rv1 cells by approximately 35%. Unfunctionalized thiourea compound 12b (CII Inhibition IC₅₀=11.88±3.3 μM) was confirmed as a probable PAINS affording just 12% reduction in 22Rv1 cell viability.

The most active 7-bromobenzothiadiazine substituted derivative, 1-phenylethylamine 24c (55% CII inhibition at 100 μM, IC₅₀=79.8±4.1 μM) reduced 22Rv1 cell survival by 70% at the same concentration after 48 hrs incubation and is the most potent derivative in the 22Rv1 cell line. Derivatives 30c, the 4-(trifluoromethoxy)benzylamine (21% CII inhibition) and 23c, the 3-Phenylpropylamine (47% CII inhibition) reduced cell survival of 22Rv1 cells by 45%, 42%, respectively, while the 4-Methoxybenzylamine (20c) (64% CII inhibition) and 4-(trifluoromethyl)benzylamine (25c) (0% CII inhibition) derivatives resulted in 41% and 34%, reduced cell survival respectively. Unfunctionalized thiourea compound 12c (55% CII Inhibition) was confirmed as a probable PAINS affording just 16% reduction in 22Rv1 cell viability (FIG. 4C).

The unsubstituted benzothiadiazine derivatives that possess no significant CII inhibition activity generally afforded no reduction of 22Rv1 cell viability. However, 1-phenylethylamine (24d) and 4-(trifluoromethoxy)benzylamine (30d) were both equipotent to reduce cell survival of 22Rv1 cells by approximately 30%. These two side chain derivatives display the greatest reduction in 22Rv1 cell viability across all four benzothiadiazine derivative classes, suggesting the 1-phenethylamine and 4-(trifluoromethoxy)benzylamine contribute to a common pharmacophore. While several novel benzothiadiazine derivatives have been identified that possess significant activity to suppress prostate cancer cell viability, potency to inhibit CII does not correlate to antineoplastic activity. Indeed, the derivatives with the greatest effect to reduce cell viability in 22Rv1 cells (FIG. 4) possess a range of CII inhibition activity from 0% (21b) to 64% (20c).

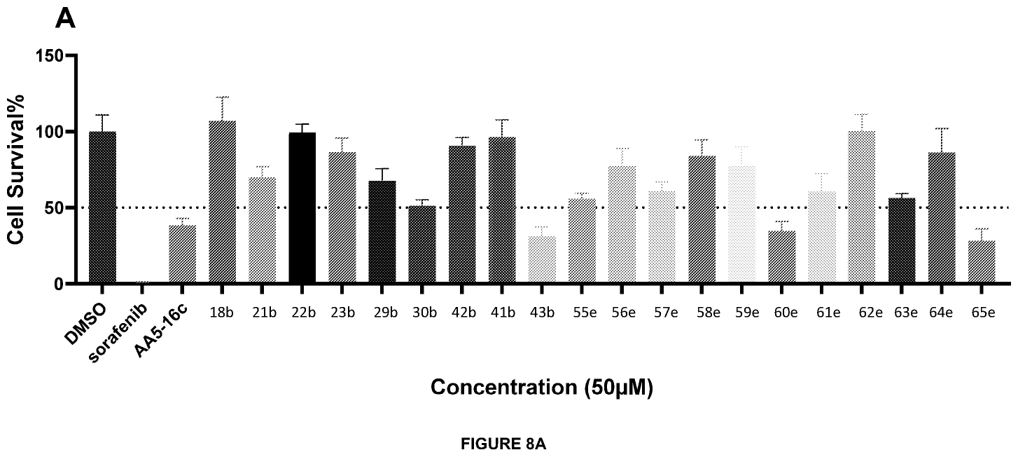

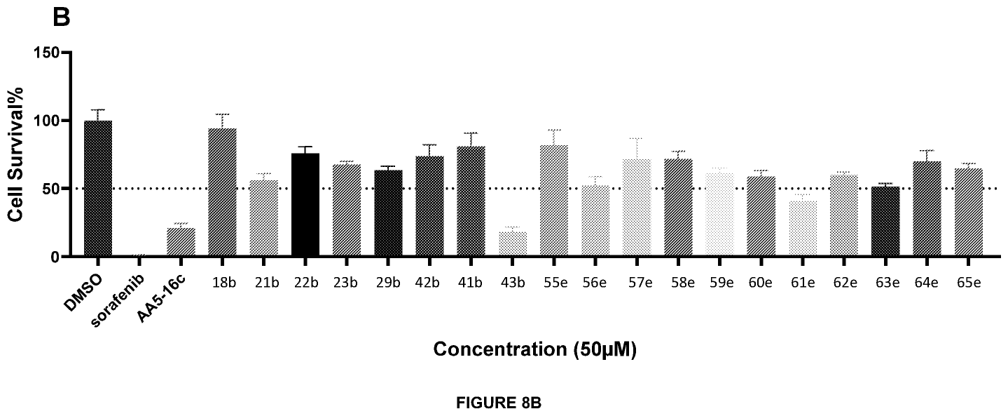

Cytotoxicity was also evaluated by using the low tumorigenic HEK293 cells at 50 μM concentration. The results showed that most of these compounds were unable to inhibit the proliferation of the HEK293 cells by more than 50% at 50 μM concentration. Three diazoxide derivatives inhibited the proliferation of HEK293 cells more than 50% with the range of inhibition between 65-72% (FIG. 8A). 6-Chloro-3-((2-(trifluoro-methoxy)benzyl)amino)-4H-benzo[e][1,2,4]thiadiazine 1,1-dioxide (43b) is the most toxic derivative of the 6-chloro substituted derivatives with an IC₅₀ of 30.48 μM. Of the diazoxide derivatives featuring 7-chloro substitution, 60e and 65e, also exhibited toxicity with IC₅₀ values of 30.85 μM and 23.41 μM, respectively (Table 4).

The potent VEFG inhibitor compounds showed modest activity at 50 μM against the most aggressive triple negative breast cancer cells (MDA-MB-468). The IC₅₀ value for the most potent compound 43b was 14.93 μM. Moreover, compound 43b showed good activity and moderate selectivity against MDA-MB-468 cancer cell lines over low tumorigenic HEK293 cells (IC₅₀=30.48 μM). The 7-chlorobenzothiadiazine derivative 61e showed the second most potent activity to reduce MDA-MB-468 cell viability with an IC₅₀ value of 43.10 μM. The 7-chlorobenzothiadiazine derivative (2,4-difluorophenyl) methanamine (63e) afforded IC₅₀=45.49 μM which had lower IC₅₀ value than the 6-chlorobenzothiadiazine derivative (29b) with IC₅₀=84.63 μM. 6-Chloro-3-((4-(trifluoromethoxy)benzyl)amino)-4H-benzo[e][1,2,4] thiadiazine 1,1-dioxide (30b) possessed an IC₅₀ value of 48.01 μM in MDA-MB-468 and IC₅₀=77.16 μM in low tumorigenic HEK293 cells (Table 4). (FIG. 8B).

Table 4 shows the cytotoxicity of the potent VEGF inhibitors of diazoxide derivatives and the clinical chemotherapeutic sorafenib in triple negative breast cancer MDA-MB-468 cells and low tumorigenic human endothelial kidney (HEK293) cells.

TABLE 4

| | | MDA-MB-468 | HEK293 |
|---|---|---|---|
| Compound | Structure | IC₅₀ (μM)^{a} | IC₅₀ (μM)^{a} |
| Sorafenib | 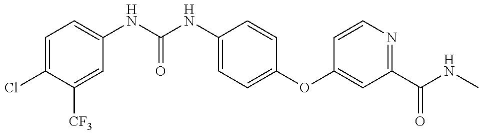 [SMILES: CNC(=O)c1cc(Oc2ccc(NC(=O)Nc3ccc(Cl)c(C(F)(F)F)c3)cc2)ccn1] | 7.31 ± 1.9 | 2.95 ± 0.1 |
| AA5-16c | 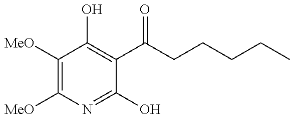 [SMILES: CCCCCC(=O)c1c(O)nc(OC)c(OC)c1O] | 6.71 ± 0.8 | 25.93 ± 2.4 |
| 21b | 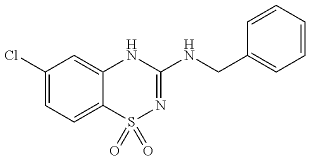 [SMILES: O=S1(=O)N=C(NCc2ccccc2)Nc2cc(Cl)ccc21] | 64.16 ± 14.2 | 101.90 ± 16.1 |
| 22b | 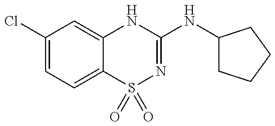 [SMILES: O=S1(=O)N=C(NC2CCCC2)Nc2cc(Cl)ccc21] | 147.80 ± 26.3 | N.D.^{b} |
| 23b | 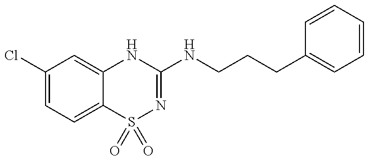 [SMILES: O=S1(=O)N=C(NCCCc2ccccc2)Nc2cc(Cl)ccc21] | 91.91 ± 16.8 | 110.81 ± 7.4 |
| 29b | 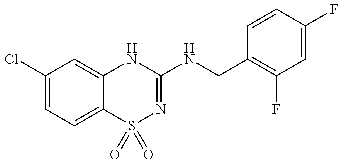 [SMILES: O=S1(=O)N=C(NCc2ccc(F)cc2F)Nc2cc(Cl)ccc21] | 84.63 ± 9.6 | 129.40 ± 12.2 |
| 30b | 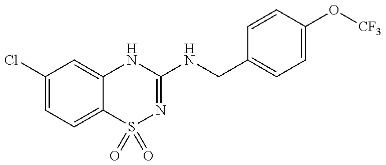 [SMILES: O=S1(=O)N=C(NCc2ccc(OC(F)(F)F)cc2)Nc2cc(Cl)ccc21] | 48.01 ± 14.3 | 77.16 ± 5.2 |
| 43b | 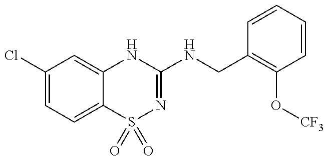 [SMILES: O=S1(=O)N=C(NCc2ccccc2OC(F)(F)F)Nc2cc(Cl)ccc21] | 14.93 ± 2.5 | 30.48 ± 4.1 |
| 56e | 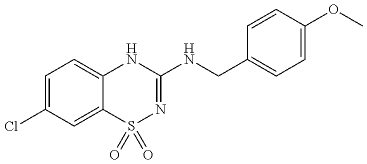 [SMILES: COc1ccc(CNC2=NS(=O)(=O)c3cc(Cl)ccc3N2)cc1] | 62.90 ± 4.2 | N.D. |
| 60e | 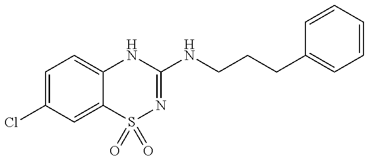 [SMILES: O=S1(=O)N=C(NCCCc2ccccc2)Nc2ccc(Cl)cc21] | N.D. | 30.85 ± 3.9 |
| 61e | 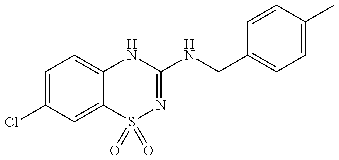 [SMILES: Cc1ccc(CNC2=NS(=O)(=O)c3cc(Cl)ccc3N2)cc1] | 43.10 ± 7.5 | N.D. |
| 63e | 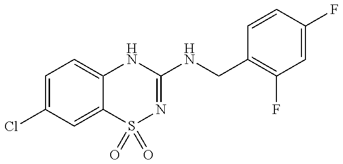 [SMILES: O=S1(=O)N=C(NCc2ccc(F)cc2F)Nc2ccc(Cl)cc21] | 45.49 ± 9.1 | 58.94 ± 13.7 |
| 65e | 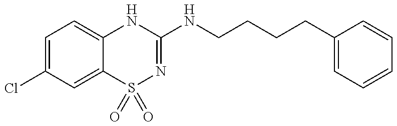 [SMILES: O=S1(=O)N=C(NCCCCc2ccccc2)Nc2ccc(Cl)cc21] | N.D. | 23.41 ± 3.2 |

^{a}Values are the mean ± SD of n = 3 experiments at 72 hours.
^{b}Not Determined

Administration of 300 mg/kg of diazoxide to rats bearing hormone-dependent mammary carcinomas was reported to result in 90% inhibition of tumor growth but induced mild reversible diabetes. Additionally, diazoxide has been reported to be cytotoxic in TNBC cells.³⁷ Based on these studies, the cytotoxic effect of selected diazoxude derivatives was explored in the MDA-MB 468 TNBC cell line. Derivatives were dosed at 10, 50 and 100 μM for 24, 48 and 72 hours and cell viability measured by MTS assay (FIG. 5).

The parent compound diazoxide, afforded little activity to reduce TNBC cell viability. Gratifyingly, several derivatives demonstrated marked dose and time-dependent reduction of cell viability; 7-fluorobenzothiadiazine derivatives 23a, 26a, 36a and 39a induced almost complete amelioration of cell viability at 100 μM concentration after 72 hours. Interestingly the 3-methylbutanamine side chain derivative (15a) was substantially less active than its aromatic side chain bearing counterparts. The 7-bromobenzothiadiazine derivatives 18c and 20c displayed potent activity but generally less so than the 7-fluorobenzothiadiazine derivatives discussed above. Unsubstituted thiourea 12a as well as unsubstituted benzothiadiazine derivatives 20d and 43d demonstrated no reduction of cell viability at any time or dose tested.

Of the most active compounds in the TNBC cells with the single exception of 26a, none show appreciable activity to inhibit CII, pointing to an as yet undetermined target of action for these novel substituted benzothiadiazines.

Exposure of human T leukemic Jurkat cells to 100 μM of diazoxide resulted in significant inhibition of proliferation; however, upon removal of the compound proliferation resumed. The study demonstrated that while diazoxide exposure depolarized the mitochondrial membrane this was insufficient to modulate cellular energy metabolism. It was found that exposure to diazoxide resulted in reduction of cellular Ca²⁺ influx. Diazoxide has further been reported to inhibit lung cancer cell proliferation by downregulating Cyclin D1 transcription.

Diazoxide has been investigated in one pilot clinical study in breast cancer patients at a dose of 200-300 mg per day. Treatment of nine patients resulted in a 33% response rate conferring stable disease for between 4-8 months either in combination with tamoxifen (two patients) or monotherapy (one patient). The repurposing of Diazoxide as a potential treatment for TNBC has been recently proposed based on a study employing a KinomeScan™ assay of 438 kinases, the three most inhibited by diazoxide at 100 μM were TTK (15%), IRAK1 (9%) and DYRK1A (7%).³⁷ Dysfunction of all three kinases are known to be associated with various cancers. As observed herein, the activity of diazoxide was highly dependent on the cell line employed; no activity was observed in MCF-7 breast cancer cells (IC₅₀=130 μM) but in MDA-MB-468 TNBC cells an IC₅₀=0.87 μM was reported. This and the previously discussed studies suggest the potential of novel diazoxide derivatives such as those identified herein to act as lead compounds for drug discovery to treat TNBC. Indeed, the potential of repurposing Diazoxide in breast cancer has been advanced with the authors suggesting combination treatment to manage the hyperglycemia ‘side effect’ of diazoxide in this context. Through the SAR studies initiated herein, medicinal chemistry modulation of the parent compound may be possible to increase antineoplastic effect while tuning out the known pharmacophore leading to the mK_{ATP} opening activity of the parent compound and the hyperglycemic effect, potential allowing access to novel treatments for a difficult to treat cancer.

### Example 4: VEGF Inhibition Assays

The assay was performed as follows. Vascular endothelial growth factor (VEGF, Cat #SRP3182) was purchased from Sigma. Diazoxide (Alfa Aesar™, Cat #AAJ66010ME) and Sorafenib™ (Tocris Bioscience™, Cat #68-141-0) were purchased from Fisher Scientific. Stock solutions of all compounds were prepared in DMSO and were serially diluted for cell culture treatment maintaining the final DMSO concentration at less than 1%. Cell proliferation of HUVECs was evaluated through the MTT (3-[4, 5-dimethylthiazol-2-yl]-2, 5-dimethyltetrazolium bromide) colorimetric assay, as previously described.²⁸ The HUVECs were seeded at a density of 10,000 cells/well in 24 well plates in serum-containing medium and cultured overnight. Cells were starved with 300 μL of assay media (0.1% BSA+0.1% FBS in basal medium) for 24 h. Then, cells were treated with 500 μL of assay medium (control group), VEGF (10 ng/mL, positive control group) or VEGF along with compounds (20 μM). After 48 h of culture, 50 μL of MTT stock (5 mg/mL in PBS) was added to each well and incubated for 2 h at 37° C. to allow the formation of dark blue formazan crystals in the metabolically active cells. The medium was removed, the cells were washed with PBS (pH 7.4), and 100 μL of acidified isopropanol (0.33 mL HCl in 100 mL isopropanol) was added to each well and incubated for 5 min with thorough agitation to solubilize the formazan crystals. An equal volume of the solution was transferred to a 96-well plate and the absorbance was immediately measured using a microplate reader at a wavelength of 570 nm. Results were confirmed by direct measurement of the cells using a standard hemocytometer.

### Western Blot Analysis

Cells were treated with test compounds (20 μM) along with VEGF (10 ng/mL) for 5 min. The DMSO was used as a negative control, whereas DMSO+VEGF was used as a positive control. Cells were lysed using RIPA buffer (10 mmol/L Tris-HCl, 1 mmol/L EDTA, 0.5 mmol/L EGTA, 1% Triton X-100, 0.1% sodium deoxycholate, 0.1% SDS and 140 mmol/L NaCl), supplemented with protease and phosphatase inhibitors (Halt Protease and Phosphatase Inhibitor). Cell lysates were centrifuged, and the supernatants were mixed with appropriate amount of 5×SDS loading buffer. Proteins were resolved on polyacrylamide gel and transferred onto nitrocellulose/PVDF membrane, blocked for 1 hour with 10% milk block prepared in 0.1% Tween 20 in 1×PBS (PBST), and treated with pVEGFR2 primary antibody at 4° C. overnight. The membrane was washed thrice with PBST, 5 minutes each, and treated with secondary antibody for 1 hour. The membrane was further washed three times, 5 minutes each, with PBST and developed using chemiluminescence, and the bands were analyzed using densitometry.

### Statistical Analysis

Experiments were repeated at least thrice, and the statistical significance was calculated using the unpaired t test. A p value of <0.05 was considered statistically significant. IC₅₀ values were calculated by GraphPad prism software.

### VEGF Inhibition Assays

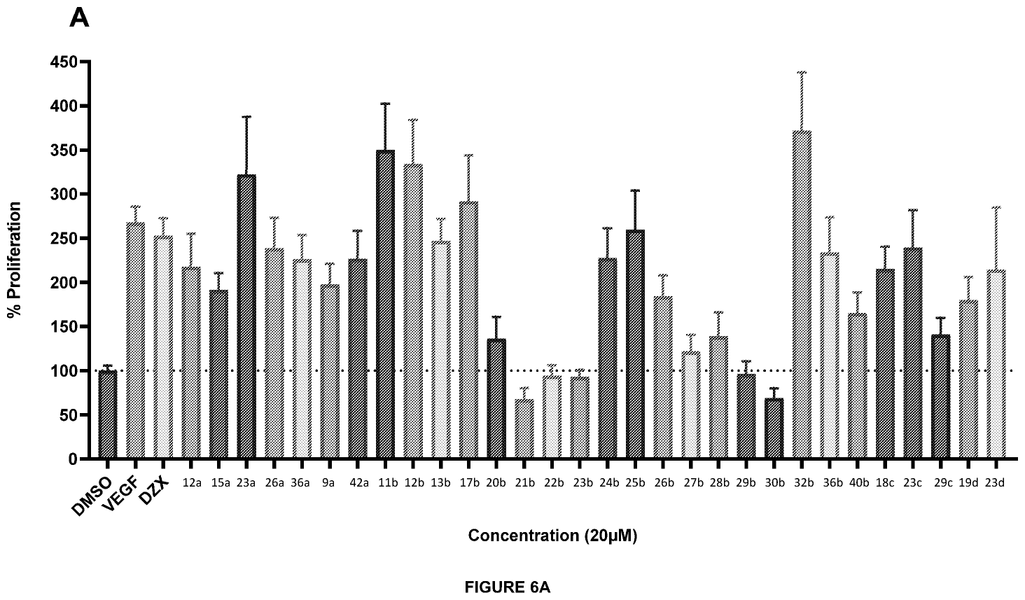

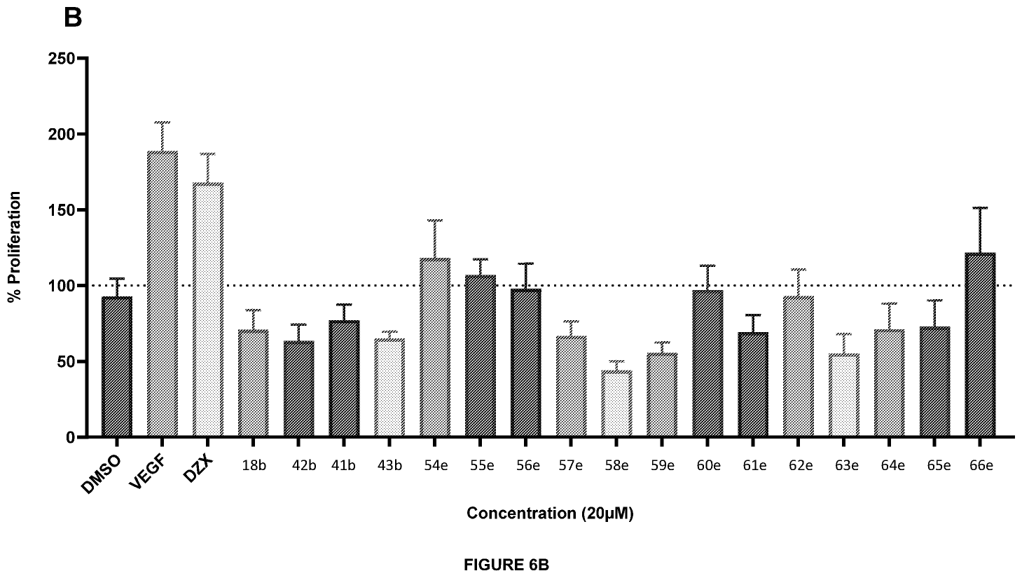

Given the reported VEGF inhibitor activity of the parent DZX compound the antiangiogenesis properties of a selection of the published DZX derivative library was evaluated, to inhibit VEGF-induced proliferation of HUVEC cells using the MTT assay. A number of compounds at 20 μM concentration were found to inhibit VEGF-induced HUVEC proliferation with high statistical significance compared to VEGF treated cells and DMSO control (FIG. 6A).

The 7-fluorobenzothiadiazine substituted derivatives (12a, 15a, 23a, 26a, 36a, 9a, and 42a) afforded no appreciable inhibition of VEGF-induced proliferation (FIG. 6A). Derivatives with a 7-bromobenzothiadiazine ring (18c, 23c, and 29c) fared little better with only 29c providing appreciable inhibition of 127% (Table 5). Interestingly 6-chlorobenzothiadiazine derivatives (11b, 12b, 13b, 17b, 20b, 21b, 22b, 23b, 24b, 25b, 26b, 27b, 28b, 29b, 32b, 36b, and 40b) generally possessed enhanced activity to inhibit VEGF-induced cell proliferation. The homologation series of unsubstituted benzylamine (21b) derivatives including phenethyl (18b), and phenyl propyl (23b) were found to possess significant activity with inhibition percentages compared to VEGF treatment alone of 200±12%, 118±12% and 178±8% respectively. This highlights a potentially important role for the 6-chlorobenzothiadiazine substituent when compared to its 7-fluorobenzothiadiazine counterpart (23a, inactive) and 7-bromobenzothiadiazine (23c, 24±41% inhibition). Further VEGF inhibitors were designed with a focus on chlorine substituted derivatives. A range of benzylamine side chains featuring various substituents on the phenyl ring were synthesized. Functionalization with an electron-donating methoxy group resulted in increased inhibition of VEGF-induced proliferation over DZX depending on location; 4-OMe (20b, 132±24%)=3-OMe (28b, 126±26)%>2-OMe (26b, 79±26%). Methyl substitution on the benzylamine ring increases inhibitory activity as shown with 4-Me (14b, 146±18%)>3-Me (21b, 125±10%)>2-Me (22b, 111±9%). Substitution with electron-withdrawing groups afforded derivatives with increased activity; 4-OCF₃ (30b, 196±11%) and 2-OCF₃ (43b, 124±4%). Interestingly the cyclopentylamine (22b, 174±12%) shows that aromaticity is not a requirement for high activity.

Most of the related 7-chlorobenzothiadiazine derivatives possessed generally lower activity to inhibit VEGF-induced proliferation, highlighted by the benzyl amine derivative (57e, 122±9% inhibition at 20 μM) and the homologation series of phenethylamine (55e, 82±10%), phenylpropylamine (60e, 92±15%), and phenylbutylamine (65e, 116±16%) not reaching the activity level of their 6-chlorobenzothiadiazine counterparts. Electron-withdrawing group substitution to the benzylamine increased activity as seen with the 6-chloro derivatives but to a lower degree, for example 2,4-difluorobenzylamine in the 7-chloro derivative 63e (133±12%) compared to the 6-chloro derivative 29b (167±14%).

DZX Derivatives Attenuate pVEGFR2 Expression

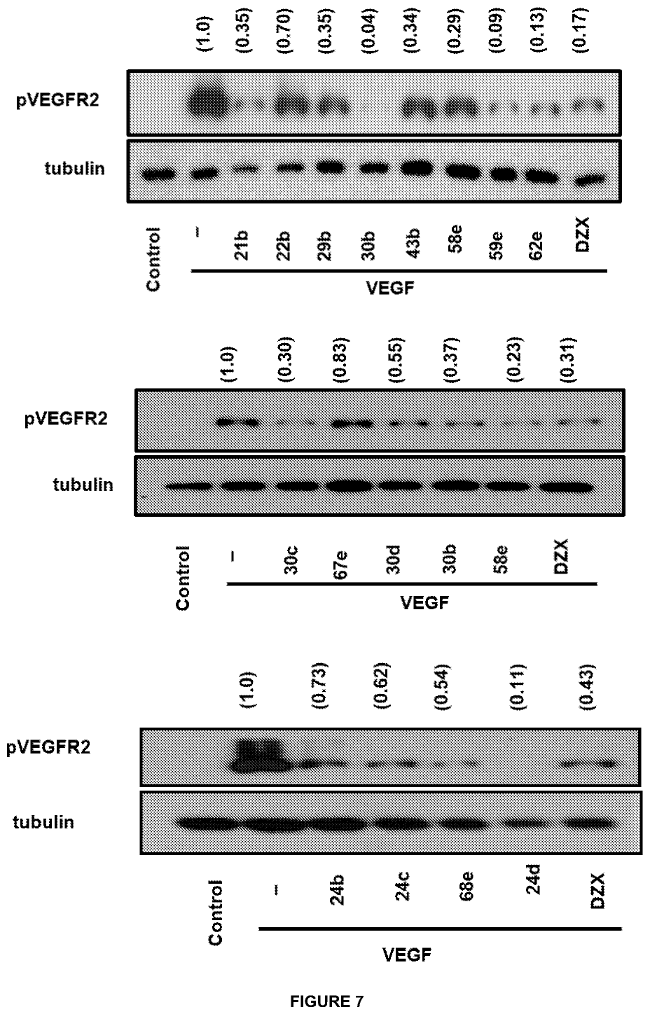

A selected number of diverse derivatives with VEGF-induced proliferation inhibition activity were investigated for their ability to modulate phosphorylation of VEGFR2 in HUVEC cells by Western blot (FIG. 7). As expected phosphorylation of VEGFR2 was highly upregulated in the presence of VEGF and completely attenuated when no VEGF was present. Treatment of selected DZX derivatives at 20 μM resulted in attenuation of expression of pVEGFR2. Diazoxide itself showed good activity to suppress pVEGFR2 while six derivatives outperformed the parent compound; 30c, 24d, 58e, 59e, 62e and 30b (FIG. 7).

Table 5 shows the structure, molecular weight, calculated log P, polar surface area, and % of proliferation inhibition relative to VEGF treated cells of diazoxide derivatives with 7-fluoro, 7-bromo substitution and a non-halogenated ring.

TABLE 5

| | | | | | % Inhibition |
|---|---|---|---|---|---|
| Compound | Structure | Mw | cLogP^{a} | PSA^{b} | of Proliferation |
| DZX | 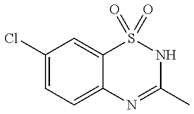 | 230.67 | 1.0 | 58.53 | 15 ± 20 |
| 12a | 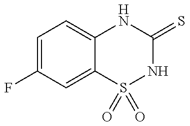 [SMILES: O=S1(=O)NC(=S)Nc2ccc(F)cc21] | 232.25 | 0.76 | 58.20 | 50 ± 35 |
| 15a | 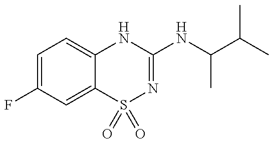 [SMILES: CC(C)C(C)NC1=NS(=O)(=O)c2cc(F)ccc2N1] | 285.34 | 1.66 | 70.56 | 76 ± 18 |
| 23a | 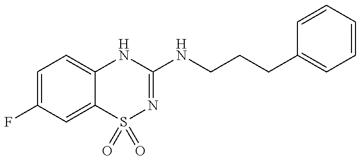 [SMILES: O=S1(=O)N=C(NCCCc2ccccc2)Nc2ccc(F)cc21] | 333.38 | 2.37 | 70.56 | 0 |
| 26a | 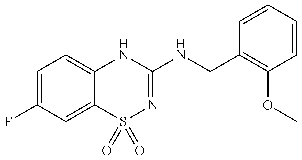 [SMILES: COc1ccccc1CNC1=NS(=O)(=O)c2cc(F)ccc2N1] | 335.35 | 1.58 | 79.79 | 29 ± 33 |
| 36a | 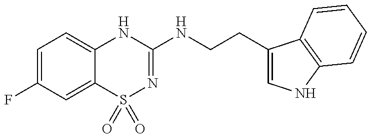 [SMILES: O=S1(=O)N=C(NCCc2c[nH]c3ccccc23)Nc2ccc(F)cc21] | 358.39 | 1.98 | 82.59 | 41 ± 26 |
| 9a | 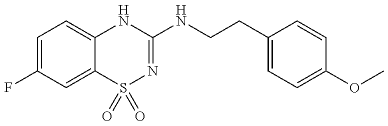 [SMILES: COc1ccc(CCNC2=NS(=O)(=O)c3cc(F)ccc3N2)cc1] | 349.38 | 1.91 | 79.79 | 70 ± 22 |
| 42a | 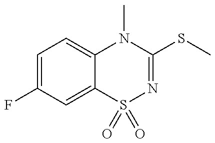 [SMILES: CSC1=NS(=O)(=O)c2cc(F)ccc2N1C] | 290.30 | 1.84 | 49.74 | 41 ± 30 |
| 18c | 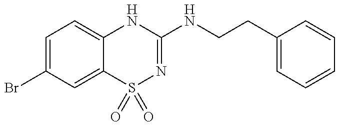 [SMILES: O=S1(=O)N=C(NCCc2ccccc2)Nc2ccc(Br)cc21] | 380.26 | 2.71 | 70.56 | 47 ± 24 |
| 23c | 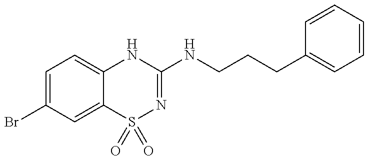 [SMILES: O=S1(=O)N=C(NCCCc2ccccc2)Nc2ccc(Br)cc21] | 394.29 | 3.09 | 70.56 | 24 ± 41 |
| 29c | 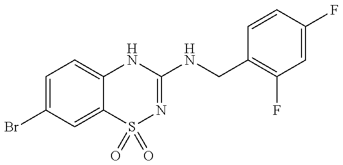 [SMILES: O=S1(=O)N=C(NCc2ccc(F)cc2F)Nc2ccc(Br)cc21] | 402.21 | 2.67 | 70.56 | 127 ± 18 |
| 30c | 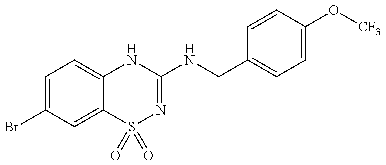 [SMILES: O=S1(=O)N=C(NCc2ccc(OC(F)(F)F)cc2)Nc2ccc(Br)cc21] | 450.23 | 3.41 | 79.79 | N.D. |
| 24c | 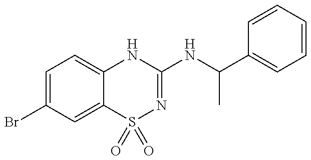 [SMILES: CC(NC1=NS(=O)(=O)c2cc(Br)ccc2N1)c1ccccc1] | 380.26 | 2.69 | 70.56 | N.D. |
| 19d | 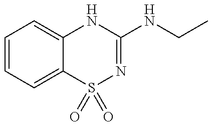 | 225.27 | 0.28 | 70.56 | 81 ± 25 |
| 23d | 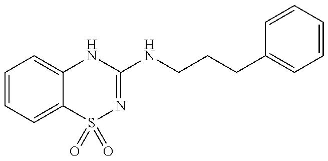 [SMILES: O=S1(=O)N=C(NCCCc2ccccc2)Nc2ccccc21] | 315.39 | 2.22 | 70.56 | 39 ± 68 |
| 30d | 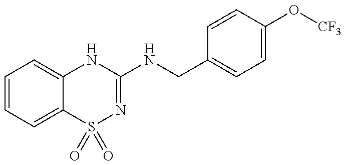 [SMILES: O=S1(=O)N=C(NCc2ccc(OC(F)(F)F)cc2)Nc2ccccc21] | 371.33 | 2.54 | 79.79 | N.D. |
| 24d | 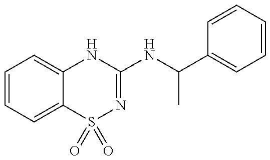 [SMILES: CC(NC1=NS(=O)(=O)c2ccccc2N1)c1ccccc1] | 301.36 | 1.83 | 70.56 | N.D. |
| 11b | 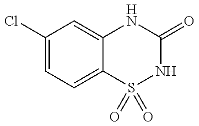 [SMILES: O=C1Nc2cc(Cl)ccc2S(=O)(=O)N1] | 232.64 | 1.52 | 75.27 | 0 |
| 12b | 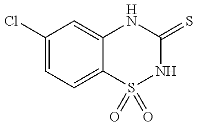 [SMILES: O=S1(=O)NC(=S)Nc2cc(Cl)ccc21] | 248.7 | 1.33 | 58.2 | 0 |
| 13b | 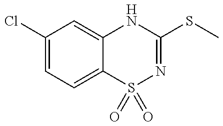 | 262.73 | 1.54 | 58.53 | 21 ± 23 |
| 17b | 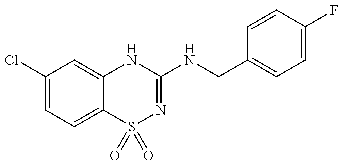 [SMILES: O=S1(=O)N=C(NCc2ccc(F)cc2)Nc2cc(Cl)ccc21] | 339.77 | 2.37 | 70.56 | 0 |
| 18b | 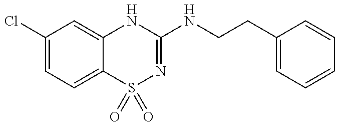 [SMILES: O=S1(=O)N=C(NCCc2ccccc2)Nc2cc(Cl)ccc21] | 335.81 | 2.56 | 70.56 | 118 ± 12 |
| 20b | 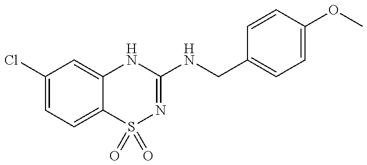 [SMILES: COc1ccc(CNC2=NS(=O)(=O)c3ccc(Cl)cc3N2)cc1] | 351.81 | 2.15 | 79.79 | 132 ± 24 |
| 21b | 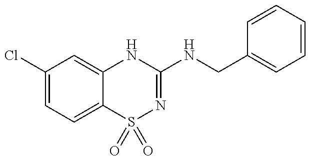 [SMILES: O=S1(=O)N=C(NCc2ccccc2)Nc2cc(Cl)ccc21] | 321.78 | 2.23 | 70.56 | 200 ± 12 |
| 22b | 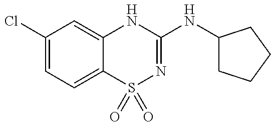 [SMILES: O=S1(=O)N=C(NC2CCCC2)Nc2cc(Cl)ccc21] | 299.77 | 1.93 | 70.56 | 174 ± 12 |
| 23b | 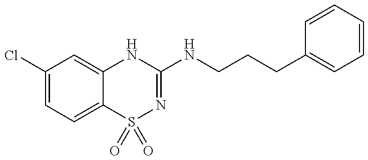 [SMILES: O=S1(=O)N=C(NCCCc2ccccc2)Nc2cc(Cl)ccc21] | 349.83 | 2.94 | 70.56 | 178 ± 8 |
| 24b | 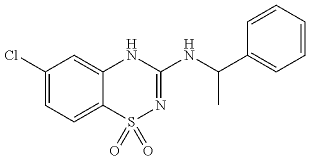 [SMILES: CC(NC1=NS(=O)(=O)c2ccc(Cl)cc2N1)c1ccccc1] | 335.81 | 2.54 | 70.56 | 38 ± 33 |
| 25b | 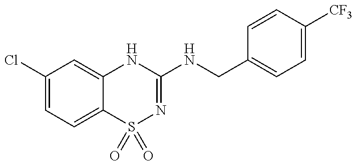 [SMILES: O=S1(=O)N=C(NCc2ccc(C(F)(F)F)cc2)Nc2cc(Cl)ccc21] | 389.28 | 3.11 | 70.56 | 1 ± 43 |
| 26b | 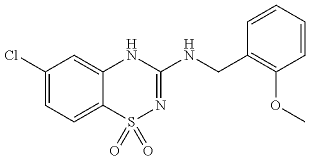 [SMILES: COc1ccccc1CNC1=NS(=O)(=O)c2ccc(Cl)cc2N1] | 351.81 | 2.15 | 79.79 | 79 ± 23 |
| 27b | 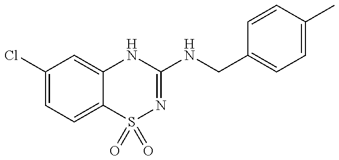 [SMILES: Cc1ccc(CNC2=NS(=O)(=O)c3ccc(Cl)cc3N2)cc1] | 335.81 | 2.73 | 70.56 | 146 ± 18 |
| 28b | 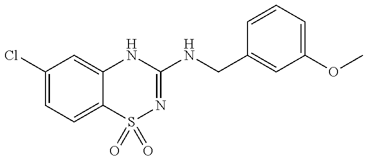 [SMILES: COc1cccc(CNC2=NS(=O)(=O)c3ccc(Cl)cc3N2)c1] | 351.81 | 2.15 | 79.79 | 126 ± 26 |
| 29b | 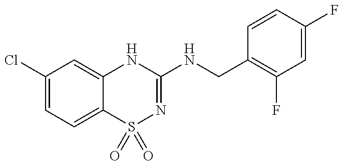 [SMILES: O=S1(=O)N=C(NCc2ccc(F)cc2F)Nc2cc(Cl)ccc21] | 357.76 | 2.52 | 70.56 | 167 ± 14 |
| 30b | 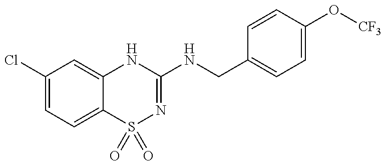 [SMILES: O=S1(=O)N=C(NCc2ccc(OC(F)(F)F)cc2)Nc2cc(Cl)ccc21] | 405.78 | 3.26 | 79.79 | 196 ± 11 |
| 32b | 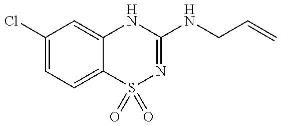 [SMILES: C=CCNC1=NS(=O)(=O)c2ccc(Cl)cc2N1] | 271.72 | 1.24 | 70.56 | 0 |
| 36b | 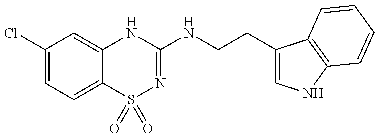 [SMILES: O=S1(=O)N=C(NCCc2c[nH]c3ccccc23)Nc2cc(Cl)ccc21] | 374.84 | 2.55 | 82.59 | 24 ± 38 |
| 40b | 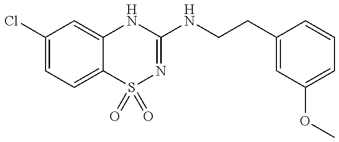 [SMILES: COc1cccc(CCNC2=NS(=O)(=O)c3ccc(Cl)cc3N2)c1] | 365.83 | 2.48 | 79.79 | 99 ± 22 |
| 42b | 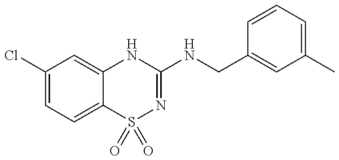 [SMILES: Cc1cccc(CNC2=NS(=O)(=O)c3ccc(Cl)cc3N2)c1] | 335.81 | 2.73 | 70.56 | 125 ± 10 |
| 41b | 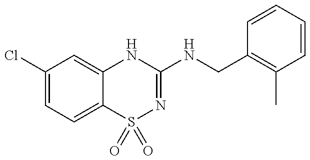 [SMILES: Cc1ccccc1CNC1=NS(=O)(=O)c2ccc(Cl)cc2N1] | 335.81 | 2.68 | 70.56 | 111 ± 9 |
| 43b | 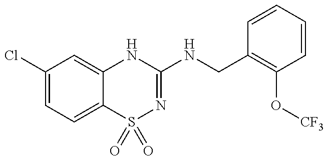 [SMILES: O=S1(=O)N=C(NCc2ccccc2OC(F)(F)F)Nc2cc(Cl)ccc21] | 405.78 | 3.26 | 79.79 | 124 ± 4 |
| 54e | 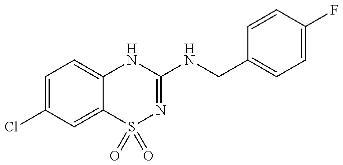 [SMILES: O=S1(=O)N=C(NCc2ccc(F)cc2)Nc2ccc(Cl)cc21] | 339.77 | 2.37 | 70.56 | 70 ± 23 |
| 55e | 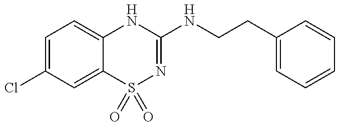 [SMILES: O=S1(=O)N=C(NCCc2ccccc2)Nc2ccc(Cl)cc21] | 335.81 | 2.56 | 70.56 | 82 ± 10 |
| 56e | 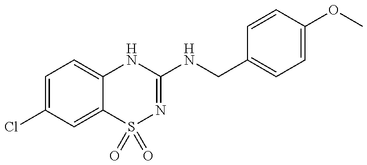 [SMILES: COc1ccc(CNC2=NS(=O)(=O)c3cc(Cl)ccc3N2)cc1] | 351.81 | 2.15 | 79.79 | 91 ± 15 |
| 57e | 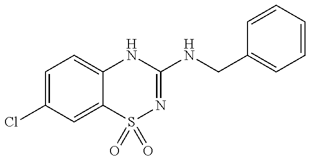 [SMILES: O=S1(=O)N=C(NCc2ccccc2)Nc2ccc(Cl)cc21] | 321.78 | 2.23 | 70.56 | 122 ± 9 |
| 58e | 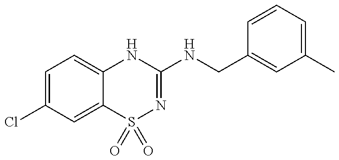 [SMILES: Cc1cccc(CNC2=NS(=O)(=O)c3cc(Cl)ccc3N2)c1] | 335.81 | 2.73 | 70.56 | 145 ± 6 |
| 59e | 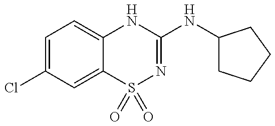 [SMILES: O=S1(=O)N=C(NC2CCCC2)Nc2ccc(Cl)cc21] | 299.77 | 1.93 | 70.56 | 133 ± 6 |
| 60e | 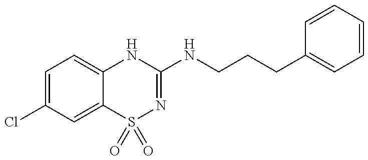 [SMILES: O=S1(=O)N=C(NCCCc2ccccc2)Nc2ccc(Cl)cc21] | 349.83 | 2.94 | 70.56 | 92 ± 15 |
| 61e | 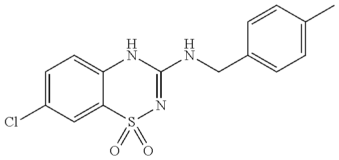 [SMILES: Cc1ccc(CNC2=NS(=O)(=O)c3cc(Cl)ccc3N2)cc1] | 335.81 | 2.73 | 70.56 | 119 ± 10 |
| 62e | 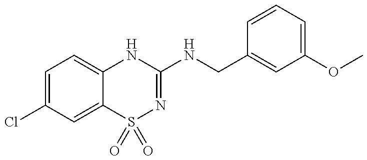 [SMILES: COc1cccc(CNC2=NS(=O)(=O)c3cc(Cl)ccc3N2)c1] | 351.81 | 2.15 | 79.79 | 96 ± 16 |
| 63e | 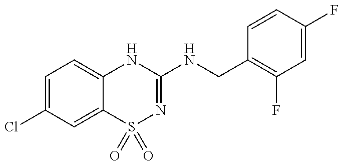 [SMILES: O=S1(=O)N=C(NCc2ccc(F)cc2F)Nc2ccc(Cl)cc21] | 357.76 | 2.52 | 70.56 | 133 ± 12 |
| 64e | 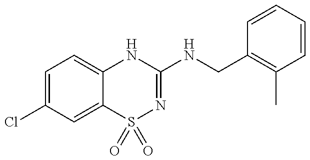 [SMILES: Cc1ccccc1CNC1=NS(=O)(=O)c2cc(Cl)ccc2N1] | 335.81 | 2.68 | 70.56 | 117 ± 15 |
| 65e | 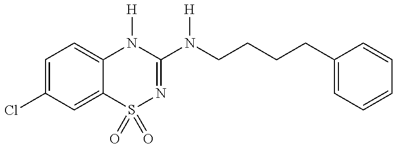 [SMILES: O=S1(=O)N=C(NCCCCc2ccccc2)Nc2ccc(Cl)cc21] | 363.86 | 3.47 | 70.56 | 116 ± 16 |
| 66e | 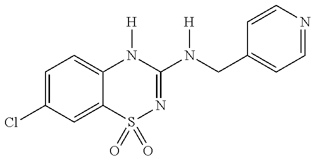 [SMILES: O=S1(=O)N=C(NCc2ccncc2)Nc2ccc(Cl)cc21] | 322.77 | 0.73 | 82.92 | 67 ± 27 |
| 67e | 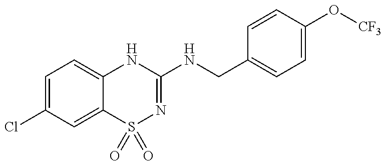 [SMILES: O=S1(=O)N=C(NCc2ccc(OC(F)(F)F)cc2)Nc2ccc(Cl)cc21] | 405.78 | 3.26 | 79.79 | N.D. |
| 68e | 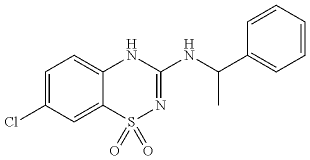 [SMILES: CC(NC1=NS(=O)(=O)c2cc(Cl)ccc2N1)c1ccccc1] | 335.81 | 2.54 | 70.56 | N.D. |

^{a}Calculated by ChemDraw Professional 16.0.
^{b}Polar surface area (pH 7.4), calculated by ChemDraw Professional 16.0.
^{c}Percentage of suppression VEGF-mediated cells growth. Values represent the mean ± SEM of n = 3 experiments.

In summary, two benzothiadiazine derivative classes have been identified (24a-d and 30a-d) that possess enhanced activity to reduce the cell viability of 22Rv1 prostate cancer cells and five novel 7-fluorobenzothiadiazine derivatives that show significant dose and time-dependent inhibition of MDA-MB-468 triple negative breast cancer cells suitable for further investigation. The CII inhibition activity of diazoxide derivatives has been shown not to be responsible for the observed cytotoxicity in either type of cancer cell line and that the cytotoxicity is selective between TNBC and prostate cancer cells with no derivatives conferencing potent cytotoxic effect in both. Thus, indicating that the derivatives are not generally toxic and engage a target not commonly expressed in MDA-MB-468 or 22Rv1 cells.

The development and use of antiangiogenic agents, especially those targeting the common growth factor VEGF, has become an integral component of antineoplastic regimens for many cancer types. Several novel benzothiadiazine derivatives 18b, 20b, 21 b, 22b, 23b, 29b, 30b, 41b, 42b, 43b, 56e, 57e, 58e, 59e, 60e, 61e, 62e, 63e, 64e, 65e, and 66e that possess enhanced activity to inhibit the HUVEC-VEGF treated cells were identified. DZX derivatives 30b, 59e, 62e and 24d were found to be most potent towards attenuation of pVEGFR2 expression. These potent derivatives reduced the cell viability of triple negative breast cancer MDA-MB-468 cells and possess much lower toxicity to low tumorigenic HEK293 cells than the clinical VEGF inhibitor sorafenib. Thus, these compound represent hits for further development to identify VEGF inhibitors with greater therapeutic index.

In view of the many possible embodiments to which the principles of the disclosure may be applied, it should be recognized that the illustrated embodiments are only examples and should not be taken as limiting the scope of the invention.

What is claimed:

1. A compound, or pharmaceutically acceptable salt thereof, having the structure of Formula I: 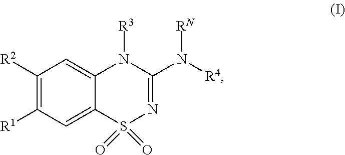
wherein
R^{N} is H or C₁₋₆ alkyl;
R¹ and R² are each independently H, halo, CN, C₁₋₆ haloalkyl, isothiocyanate, or —OSO₂R^{S}, wherein R^{S} is C₁₋₃ alkyl, C₁₋₃ haloalkyl, or phenyl optionally substituted with one of halo, C₁₋₃ alkyl, or NO₂;
R³ is H, C₁₋₆ alkyl, C₃₋₁₀ cycloalkyl, or C₆₋₁₀ aryl;
R⁴ is C₀₋₆alkylene-C₃₋₆ cycloalkyl, C₀₋₆alkylene-3-12 membered heterocycloalkyl having 1-3 ring heteroatoms selected from O, S, and N, C₀₋₆alkylene-C₆₋₁₀ aryl, or C₀₋₆alkylene-5-10 membered heteroaryl having 1-4 heteroatoms selected from N, O, and S, and said cycloalkyl, heterocycloalkyl, aryl, and heteroaryl are optionally substituted with one or more R⁵; and
each R⁵ is independently halo, C₁₋₆ alkyl, C₁₋₆ haloalkyl, C₁₋₆ alkoxy, C₁₋₆ haloalkoxy, S—C₁₋₆ haloalkyl, or SO₂N(R^{N})₂,
with the proviso that the compound is not:
3-(cyclopentylamino)-7-fluoro-4H-benzo[e][1,2,4]thiadiazine 1,1-dioxide;
6-chloro-3-(cyclopentylamino)-4H-benzo[e][1,2,4]thiadiazine 1,1-dioxide; or
6-chloro-3-(phenylamino)-4H-benzo[e][1,2,4]thiadiazine 1,1-dioxide.

2. The compound or salt of claim 1, wherein R^{N} is H.

3. The compound or salt of claim 1, wherein R^{N} is C₁₋₆ alkyl.

4. The compound or salt of claim 3, wherein R^{N} is methyl.

5. The compound or salt of any one of claims 1 to 4, wherein R³ is H.

6. The compound or salt of any one of claims 1 to 5, wherein R¹ is H, halo, or CN.

7. The compound or salt of claim 6, wherein R¹ is H, F, Br, or CN.

8. The compound or salt of claim 7, wherein R¹ is H, F, or Br.

9. The compound or salt of claim 6, wherein R¹ is H or Cl.

10. The compound or salt of any one of claims 6 to 9, wherein R¹ is H.

11. The compound or salt of any one of claims 6 to 8, wherein R¹ is F or Br.

12. The compound or salt of claim 11, wherein R¹ is F.

13. The compound or salt of claim 11, wherein R¹ is Br.

14. The compound or salt of claim 9, wherein R¹ is Cl.

15. The compound or salt of any one of claims 1 to 14, wherein R² is H, halo, or CN.

16. The compound or salt of claim 15, wherein R² is H, Cl, or CN.

17. The compound or salt of claim 15 or 16, wherein R² is H or Cl.

18. The compound or salt of claim 17, wherein R¹ is H, F, or Br and R² is H or Cl.

19. The compound or salt of any one of claims 15 to 18, wherein R² is H.

20. The compound or salt of claim 19, wherein R¹ is F and R² is H.

21. The compound or salt of claim 20, wherein R¹ is Cl and R² is H.

22. The compound or salt of claim 20, wherein R¹ is H and R² is H.

23. The compound or salt of any one of claims 15 to 18, wherein R² is Cl.

24. The compound or salt of claim 23, wherein R¹ is H and R² is Cl.

25. The compound or salt of any one of claims 1 to 24, wherein R⁴ is C₀alkylene-C₃₋₆ cycloalkyl or C₀alkylene-C₆₋₁₀ aryl.

26. The compound or salt of claim 25, wherein R⁴ is cyclopentyl, phenyl, or naphthyl.

27. The compound or salt of claim 26, wherein R⁴ is phenyl.

28. The compound or salt of claim 26, wherein R⁴ is cyclopentyl.

29. The compound or salt of any one of claims 1 to 28, wherein R⁴ is C₁₋₆alkylene-C₃₋₆ cycloalkyl, C₁₋₆alkylene-3-12 membered heterocycloalkyl having 1-3 ring heteroatoms selected from O, S, and N, C₁₋₆alkylene-C₆₋₁₀ aryl, or C₁₋₆alkylene-5-10 membered heteroaryl having 1-4 heteroatoms selected from N, O, and S.

30. The compound or salt of any one of claims 1 to 29, wherein R⁴ is unsubstituted.

31. The compound or salt of claim 30, wherein R⁴ is C₁₋₆alkylene-C₆₋₁₀ aryl or C₁₋₆ alkylene-5-10 membered heteroaryl.

32. The compound or salt of claim 30 or 31, wherein R⁴ is C₁₋₆alkylene-phenyl, C₁₋₆alkylene-pyridinyl, or C₁₋₆alkylene-indolyl.

33. The compound or salt of claim 32, wherein R⁴ is C₁₋₄alkylene-phenyl, C₁₋₄ alkylene-pyridinyl, or C₁₋₄alkylene-indolyl.

34. The compound or salt of claim 33, wherein R⁴ is

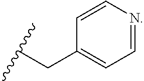 [SMILES: *Cc1ccncc1]

35. The compound or salt of claim 33, wherein R⁴ is

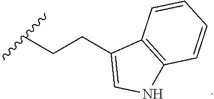 [SMILES: *CCc1c[nH]c2ccccc12]

36. The compound or salt of any one of claims 30 to 35, wherein R⁴ is C₁₋₄alkylene-phenyl.

37. The compound or salt of claim 36, wherein R⁴ is

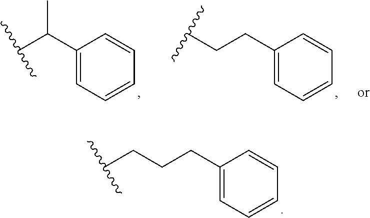

38. The compound or salt of any one of claims 1 to 29, wherein R⁴ is substituted with one or more R⁵.

39. The compound or salt of claim 38, wherein R⁴ is C₁₋₄alkylene-C₆₋₁₀ aryl.

40. The compound or salt of 37 or 39, wherein R⁴ is C₁₋₄alkylene-phenyl.

41. The compound or salt of any one of claims 38 to 40, wherein R⁴ is substituted with one R⁵.

42. The compound or salt of any one of claims 38 to 40, wherein R⁴ is substituted with two R⁵.

43. The compound or salt of any one of claims 38 to 41, wherein each R⁵ is halo, C₁₋₆ alkyl, C₁₋₆ haloalkyl, C₁₋₆ alkoxy, or C₁₋₆ haloalkoxy.

44. The compound or salt of claim 43, wherein each R⁵ is F, Cl, methyl, trifluoromethyl, methoxy, or trifluoromethoxy.

45. The compound or salt of any one of claims 38 to 41, wherein each R⁵ is halo.

46. The compound or salt of claim 45, wherein R⁴ is

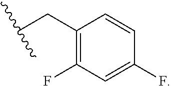 [SMILES: *Cc1ccc(F)cc1F]

47. The compound or salt of any one of claims 38 to 41, wherein each R⁵ is C₁₋₆ alkyl.

48. The compound or salt of claim 47, wherein R⁴ is

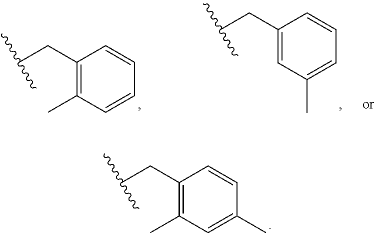

49. The compound or salt of claim 43 or 44, wherein each R⁵ is C₁₋₆ alkoxy or C₁₋₆ haloalkoxy.

50. The compound or salt of claim 49, wherein each R⁵ is methoxy or trifluoromethoxy.

51. The compound or salt of claim 50, wherein R⁴ is

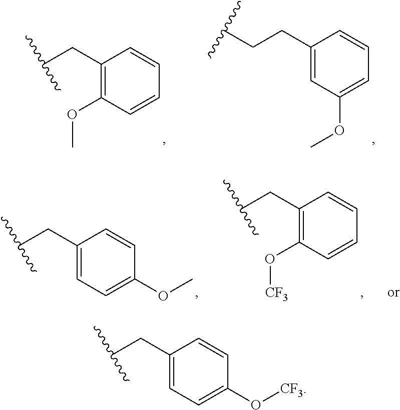

52. A compound as recited in Table 1, or a pharmaceutically acceptable salt thereof.

53. The compound or salt of claim 52, selected from the group consisting of compound 21b, 22b, 29b, 30b, 43b, 58e, 59e, 62e, 30c, 67e, 30d, 24b, 24c, 68e, 24d.

54. A pharmaceutical composition comprising the compound or salt of any one of claims 1 to 53 and a pharmaceutically acceptable carrier or excipient.

55. A method of inhibiting a vascular endothelial growth factor (VEGF) comprising administering to a subject in need thereof a therapeutically effective amount of the compound or salt of any one of claims 1 to 53 or a compound as recited in Table 2, or a pharmaceutically acceptable salt thereof, or the pharmaceutical composition of claim 54.

56. A method of treating or preventing a disease or disorder capable of being modulated by vascular endothelial growth factor (VEGF) inhibition, comprising administering to a subject in need thereof a therapeutically effective amount of the compound or salt of any one of claims 1 to 53 or a compound as recited in Table 2, or a pharmaceutically acceptable salt thereof, or the pharmaceutical composition of claim 54.

57. The method of claim 55 or 56, wherein the VEGF is vascular endothelial growth factor 2 (VEGF2).

58. The method of any one of claims 55 to 57, wherein the disease or disorder is cancer.

59. The method of claim 58, wherein the cancer is central nervous system (CNS) cancer, prostate cancer, or breast cancer.

60. The compound, salt, or composition for use of claim 59, wherein the cancer is prostate cancer or breast cancer.

61. The compound, salt, or composition for use of claim 60, wherein the disease or disorder is triple negative breast cancer.

62. The method of any one of claims 55 to 57, wherein the disease or disorder is an eye disease or disorder.

63. The method of claim 62, wherein the eye disease or disorder is neovascular age-related degeneration, retinal vascular disease, coats disease, wet macular degeneration, or submacular hemorrhage.

64. A method of treating or preventing cancer, comprising administering to a subject in need thereof the compound or salt of any one of claims 1 to 53 or a compound as recited in Table 2, or a pharmaceutically acceptable salt thereof, in an amount sufficient to be cytotoxic to a cancer cell, or the pharmaceutical composition of claim 54.

65. The method of claim 64, wherein the disease or disorder is a central nervous system (CNS) cancer, prostate cancer, or breast cancer.

66. The method of claim 65, wherein the disease or disorder is prostate cancer or breast cancer.

67. The method of claim 66, wherein the disease or disorder is triple negative breast cancer.

68. The compound or salt of any one of claims 1 to 53 or a compound as recited in Table 2, or the pharmaceutical composition of claim 54, for use in inhibiting a vascular endothelial growth factor (VEGF).

69. The compound or salt of any one of claims 1 to 53 or a compound as recited in Table 2, or the pharmaceutical composition of claim 54, for use in treating or preventing a disease or disorder capable of being modulated by vascular endothelial growth factor (VEGF) inhibition.

70. The compound, salt, or composition for use of claim 68 or 69, wherein the VEGF is vascular endothelial growth factor 2 (VEGF2).

71. The compound, salt, or composition for use of any one of claims 68 to 70, wherein the disease or disorder is cancer.

72. The compound, salt, or composition for use of claim 70 or 71, wherein the cancer is central nervous system (CNS) cancer, prostate cancer, or breast cancer.

73. The compound, salt, or composition for use of claim 72, wherein the cancer is prostate cancer or breast cancer.

74. The compound, salt, or composition for use of claim 73, wherein the disease or disorder is triple negative breast cancer.

75. The compound, salt, or composition for use of claim 68 or 69, wherein the disease or disorder is an eye disease or disorder.

76. The compound, salt, or composition of claim 75, wherein the eye disease or disorder is neovascular age-related degeneration, retinal vascular disease, coats disease, wet macular degeneration, or submacular hemorrhage.

77. The compound or salt of any one of claims 1 to 53 or a compound as recited in Table 2, or the pharmaceutical composition of claim 54, for use in treating or preventing cancer, wherein the compound or salt is present in an amount sufficient to be cytotoxic to a cancer cell.

78. The compound, salt, or composition for use of claim 77, wherein the cancer is central nervous system (CNS) cancer, prostate cancer, or breast cancer.

79. The compound, salt, or composition for use of claim 78, wherein the cancer is prostate cancer or breast cancer.

80. The compound, salt, or composition for use of claim 79, wherein the disease or disorder is triple negative breast cancer.
